(19) United States
(12) Patent Application Publication
(10) Pub. No.: US 2021/0024615 A1
(43) Pub. Date: Jan. 28, 2021

(54) CHIMERIC FC RECEPTOR BINDING PROTEINS AND USES THEREOF

(71) Applicant: LIVERPOOL SCHOOL OF TROPICAL MEDICINE, Liverpool (GB)

(72) Inventors: Richard John Pleass, Liverpool (GB); Patricia Ann Blundell, Liverpool (GB)

(21) Appl. No.: 16/981,111

(22) PCT Filed: Mar. 15, 2019
(86) PCT No.: PCT/GB2019/050744

§ 371 Date: Sep. 15, 2020

(30) Foreign Application Priority Data
Mar. 16, 2018 (GB) 1804278.8
Oct. 12, 2018 (GB) 1816635.5

(51) Int. Cl. C07K 16/00

(52) U.S. Cl. CPC C07K 16/00; C07K 2317/53

(57) ABSTRACT

The present invention relates to chimeric proteins, to compositions comprising such proteins and to the medical uses of such proteins and compositions. In particular the proteins or compositions of the invention may be used in the prevention or treatment of autoimmune diseases or inflammatory diseases, or for the prevention or treatment of diseases mediated through binding of sialic acid dependent receptors, or as vaccines or as anti-cancer agents. One aspect of the invention relates to a chimeric Fc receptor binding protein which comprises two chimeric polypeptide chains, wherein each chimeric polypeptide chain comprises an immunoglobulin G heavy chain constant region, a tailpiece region and a hinge region, wherein the amino acid sequence of each polypeptide chain possess a sugar moiety at or close to the N-terminus and a sugar moiety at or close to the C-terminus, and their use in the treatment or prevention of a disease mediated by a pathogen that relies on sialic acid receptors interactions.

### FIELD OF THE INVENTION

The present invention relates to proteins, and compositions comprising such proteins. The invention also relates to the medical uses of such proteins and compositions. In particular the proteins or compositions of the invention may be used in the prevention or treatment of autoimmune diseases or inflammatory diseases, or for the prevention or treatment of diseases mediated through binding of sialic acid dependent receptors, or as vaccines or as anti-cancer agents. The invention further relates to methods of preventing or treating autoimmune or inflammatory diseases, or diseases mediated through binding of sialic acid dependent receptors, using such proteins. The invention also relates to nucleic acids encoding the proteins, as well as methods of manufacturing the proteins.

### BACKGROUND

Autoimmune diseases (ADs) are common and affect 50 million American citizens alone. Intravenous immunoglobulin (IVIG) treatment involves the administration of purified immunoglobulin G, and is one of the most common treatments of ADs, with Food and Drug Administration (FDA) approval for a diverse range of diseases like idiopathic thrombocytopenia (ITP), Kawasaki disease, Guillain-Barré, dermatomyositis, and chronic inflammatory demyelinating polyneuropathy.

The worldwide consumption of IVIG has increased over 300 fold since 1980 and currently 100 ton are consumed per annum. Supplies of IVIG within the NHS and globally are critically limited, meaning that patients with an urgent need for the drug are routinely deprived of it. There are also significant clinical limitations resulting from its dependence on human donors for manufacture, and from the fact that <5% of injected IVIG (correctly glycosylated and/or oligomeric-Fc) is therapeutically active leading to a requirement for high doses (2 g/kg) when used in idiopathic thrombocytopenic purpura (ITP). Consequently, IVIG is expensive and adverse events due to excessive protein loading not uncommon.

Whereas some effector mechanisms of IgG relevant to autoimmune diseases may be F(ab′)2-mediated, e.g. blocking/neutralization of receptors, cytokines, anaphylatoxins and pathogenic autoantibodies via anti-idiotypic interactions, many anti-inflammatory functions are thought to be mediated by the Fc portion. They include FcRn saturation, blockade and modulation of FcγR expression, modulation of dendritic cell, B cells and T regulatory cell function and blockade/scavenging of complement components. IVIG suppresses harmful inflammation by engaging low-affinity inhibitory receptors and by forming immune-complexes (ICs) and/or dimers when injected in vivo that allow IVIG to interact with these receptors with greater strength (avidity), thus mediating more potent anti-inflammatory effects.

The problems noted above have led to a number of attempts to generate artificial agents, capable of expression on a large scale that can be used as replacements for human IgG in therapies, such as IVIG, for use in the treatment of autoimmune and inflammatory diseases.

Examples of such artificial agents that have been described to date include “SIFs” (selective immunomodulators of Fc-receptors), such as SIF-3, manufactured by Momenta, “stradomers” manufactured by Pfizer, and “hexa-Fc” an immunoglobulin-based hybrid protein produced by the current inventors. Each of the molecules produced in this manner has been designed to promote the formation of oligomeric structures that incorporate binding domains that bind to classical and non-classical Fc-receptors, including FcγRI, FcγRIIA, FcγRIIB, FcγRIIIA, FcRL5 and non-classical receptors, including DC-SIGN with high avidity.

Cells also carry various receptors that depend upon glycans comprising sialic acid for their binding. Examples of such sialic acid dependent receptors include SIGLEC-1 and SIGLEC-2. It is known that a range of diseases are mediated through binding to these sialic acid dependent receptors. For example, a number of infectious agents, such as retroviruses, bind to cells, and thus cause their associated infections, through binding to the cells' sialic acid dependent receptors. These receptors are also clinically implicated in the control of neuropathology.

Multiple lines of evidence have shown that glycosylation is critical to driving either the anti- or pro-inflammatory capability of IgG⁶⁵. Glycosylation of the only available carbohydrate attachment site (Asn-297) in the Fc is essential for interactions with type 1 receptors (Fcγ) and type 2 receptors (glycan dependent), but also for driving interactions with the complement cascade^{30,2,66}.

In humans, infusion of Fc-fragments is sufficient to ameliorate idiopathic thrombocytopenic purpura (ITP) in children, demonstrating the therapeutic utility of the Fc in vivo 5. These anti-inflammatory properties of the Fc are lost after deglycosylation of IgG, and a population of IgG-bearing sialylated Fcs has been identified as making a significant contribution to the control of inflammation in animal models^{6,7}. Higher levels of sialylation also leads to longer serum retention times^{8,9}, and studies in humans and mice have shown that influx and efflux of IgG into the central nervous system (CNS) is glycan and sialic acid dependent¹⁰⁻¹⁵.

Consequently, the efficacy of sialylated Fe has generated an incentive to modify the existing glycans on Asn297, either by chemical means or through mutagenesis programs on the Fc protein backbone that disrupt the protein-Asn297-carbohydrate interface (16-18). However, chemical modification of pre-existing glycans is expensive and reliant on a sustainable source of human Fc, while mutagenesis approaches on the Fc, or expression in glycosidase-deficient/transgenic cell lines, have yielded little improvement in Asn297 sialylation to the levels required for significant enhancements in the affinity of binding to FcγRs^{17,18}. Recently, co-administration of two glycosyltransferase Fc-fusion proteins has been shown to convert endogenous IgG into sialylated anti-inflammatory IgGs that attenuate autoimmune disease in animal models in a platelet-dependent manner¹⁹.

Although in vivo enzymatic sialylation may circumvent many technical issues concerned with chemical or mutagenic approaches to generating sialylated IgG, it may not be appropriate in all clinical settings for example in neurological diseases (e.g. neuromyelitis optica), where the target site is mostly devoid of platelets, and where two different Fc fusions would need to traverse the blood-brain barrier simultaneously. This approach also runs the risk of off-target glycan modifications and known immunogenicity of long-term administration of Fc fusions²⁰.

Mutagenesis studies to date have also been limited in two further respects. Side-chain changes have typically been restricted to alanine or serine, and, functionality studies have mostly been confined to FcγR binding studies^{21, 22} It is therefore of academic interest and potential clinical value to explore more thoroughly how the introduction of additional N-glycan sites into the Fc might affect changes in binding to FcγR and other atypical Fc-glycan receptors, including Siglecs and C-type lectins.

The human IgG1-Fc typically does not bind glycan receptors because the glycan attached to Asn-297 is largely buried within the cavity formed by the CH2-CH3 homodimer^{26,27}. The location and content of glycans attached at Asn-297 also modulates the affinity of the Fc for binding to the classical FcγRs, through conformational changes imparted to the FcγR binding region located in the lower hinge²⁸. Herein, the inventors show that these limitations to Asn-297-directed receptor binding can be overcome through a program of mutagenesis aimed at disrupting disulphide bonding while enhancing N-linked glycosylation within the IgG1-Fc.

### SUMMARY OF THE INVENTION

According to a first aspect of the invention there is provided a chimeric Fc receptor binding protein comprising two chimeric polypeptide chains; wherein each chimeric polypeptide chain comprises a hinge region, an immunoglobulin G heavy chain constant region and an immunoglobulin tailpiece region, wherein the amino acid sequence of each polypeptide chain is altered as compared to the native sequences from which the immunoglobulin G heavy chain constant region and the immunoglobulin tailpiece region are derived to remove cysteine residues that form extrinsic disulphide bonds, and wherein each polypeptide chain comprises at least one glycosylation site. These proteins are herein referred to as the “cysteine residue molecules/proteins” or “cysteine mutants”. By extrinsic disulphide bond is meant one that is formed between distinct polypeptide chains (such as an inter-chain disulphide bond with the companion polypeptide chain or another polypeptide chain, such as one from a different monomer). It does not cover cysteine residues that form intra-chain disulphide bonds. For the avoidance of doubt, it is apparent that cysteines that form inter-chain linkages may be present in the hinge region, indeed this is one way that keeps the two polypeptide chains together.

Suitably, the protein of the invention lacks the cysteine at a residue which corresponds to residue 248 of SEQ ID NO:1 (which in turn, corresponds to residue 575 of human IgM tail region).

Suitably, the protein of the invention lacks a cysteine residue at position 89 of SEQ ID NO:1 found in Hexa-Fc molecules (which position corresponds to residue 309 of human IgG).

Suitably, the protein of the invention lacks a cysteine residue at each position which correspond to residues 89 and 248 of SEQ ID NO:1.

Suitably, the protein of the invention comprises an additional glycosylation site in one of the regions that is not present in the native sequence from which the region is derived. In one embodiment, the additional glycosylation site is in the hinge region. Suitably, the additional glycosylation site is located at the amino terminal end of the hinge region. Suitably, the additional glycosylation site in the hinge region is at a residue which corresponds to residue 1 of SEQ ID NO:1 (which in turn, corresponds to residue 221 of the human IgG hinge region).

By “glycosylation site” is meant an amino acid that can accommodate attachment of a glycan, such as a sugar. Glycosylation is a form of co-translational and post-translational modification. Glycans serve a variety of structural and functional roles in membrane and secreted proteins. In N-linked glycosylation, the sugar is attached through a nitrogen (NH) of an asparagine or arginine side-group on the protein. In O-linked glycosylation, the sugar is attached through an oxygen on a hydroxyl group (OH) to a serine, threonine, or tyrosine. The N-linked glycosylation sequon is specified by Asn-X-Thr/Ser whereby X is any amino acid other than proline. There is no O-linked amino acid consensus sequence. There are no O-linked attached glycans in the Fc of native IgG1. However, the hinge sequence of IgA1 can be O-glycosylated at serines or threonines. These O-linked glycans can therefore be terminally sialylated.

Suitably, a glyosylation site of a protein of the invention is a nitrogen of an asparagine or arginine side-chain to which N-linked glycans are or can be attached.

According to a second aspect of the invention there is provided a chimeric Fc receptor binding protein comprising two chimeric polypeptide chains; wherein each chimeric polypeptide chain comprises an immunoglobulin G heavy chain constant region, an immunoglobulin tailpiece region and a hinge region, wherein the amino acid sequence of each polypeptide chain possess a sugar moiety at or close to the N-terminus (such Asn-221) and a sugar moiety at or close to the C-terminus (such as Asn563 or Asn573). Such molecules are herein also referred to as “haemagglutinin binding proteins” or “double sugar molecules”. By close to the N- or C-termini we mean within about 12 residues, such as within 8 residues, within 5 residues, or within 3 residues of a terminus. In a particular embodiment, the amino terminal sugar moiety is attached to an amino acid located at position +1 to +12, inclusive. In a particular embodiment, the carboxy terminal sugar moiety is attached to an amino acid located at position −12 to −3, inclusive. By position +1 to +12, inclusive, mean the amino acid residue is positioned from the first to twelfth residue inclusive from the N-terminus of the polypeptide. By −12 to −3 inclusive we mean the amino acid residue is positioned from the 12^{th} to 3rd residue inclusive from the C-terminus of the polypeptide.

According to a third aspect of the invention there is provided a chimeric Fc receptor binding protein comprising two chimeric polypeptide chains; wherein each chimeric polypeptide chain comprises an immunoglobulin G heavy chain constant region and an immunoglobulin tailpiece, wherein the amino acid sequence of each polypeptide chain is altered as compared to the native sequences from which the immunoglobulin G heavy chain constant region and the immunoglobulin tailpiece region are derived to possess at least two fewer glycosylation sites. Such proteins are herein also referred to as “glycosylation adapted proteins”.

According to a fourth aspect of the invention there is provided a composition comprising a protein according to the first, second or third aspect of the invention or another protein of the invention. In a particular embodiment, wherein at least 50%, such as at least 75% or at least 95% of the protein of the first, second or third aspect of the invention or another protein of the invention incorporated in the composition is in monomeric form. Suitably, the composition is a pharmaceutically acceptable formulation that comprises a protein of the invention and at least one pharmaceutically acceptable carrier.

According to a fifth aspect of the invention there is provided a protein in accordance with the first, second or third aspects of the invention or another protein of the invention for use as a medicament. The protein for use in the fifth aspect of the invention may be provided in the form of a composition in accordance with the second aspect of the invention.

Proteins or compositions of the invention may be used as medicaments in the prevention and/or treatment of autoimmune or inflammatory diseases. Suitable examples of such diseases are considered elsewhere in the specification.

Alternatively, proteins or compositions of the invention may be used as medicaments in the prevention and/or treatment of diseases mediated through the binding of sialic acid dependent receptors. In an embodiment, the receptor may be selected from the group consisting of: SIGLEC-1, SIGLEC-2, SIGLEC-3, SIGLEC-4, CD23, dec-1, dec-2, DC-SIGN, CLEC-4A, CLEC-4D, MBL, MMR and DEC-205. Suitable examples of such diseases include retroviral infections, as considered elsewhere in the specification. In an embodiment, the receptor may be selected from the group consisting of: SIGLEC-1, SIGLEC-2, SIGLEC-3 and SIGLEC-4.

Alternatively, proteins or compositions of the invention, particularly those according to the second aspect of the invention, may be used as medicaments in the prevention and/or treatment of diseases (including viral infections) mediated through the binding of haemagglutinin receptor. The molecules of the invention that possess a sugar moiety at or close to the N-terminus (such Asn-221) and a sugar moiety at or close to the C-terminus (such as Asn 563 or Asn 573) may be useful as pathogen blockers, particularly to block sialic acid-dependent pathogens, for example sialic acid-dependent viruses such as influenza (Flu) and Newcastle disease virus. The “haemagglutinin binding proteins” are particularly suitable for use in the treatment or prevention of a disease mediated by pathogens that rely on sialic acid receptor interactions.

The inventors have found that the C575A mutant (containing two sugars including tail sugar at Asn563) did not inhibit haemagglutination as well as D221N/C575A (with all sugars present).

Viruses, such as Flu, are constantly mutating and developing resistance to vaccines, such as neutralising antibodies. However, mutations in the glycan binding site are unlikely to be accommodated, or if they are accommodated they would only arise at very low frequency, because such mutations would inhibit an important virus function, e.g. entry into a host cell. Viruses that cannot gain entry into a host cell are likely to be cleared by the immune system. Molecules capable of blocking these sialic acid receptors may therefore serve as pathogen blockers.

Sialylated carbohydrates are ubiquitously expressed among vertebrates and are engaged by numerous pathogens including parainfluenza viruses, paramyxoviruses, orthoreoviruses, human SARS (CoVs), noroviruses, enteroviruses, rotaviruses and adenoviruses.

These glycans contain sialic acids, which are usually found at the termini of the branches of N-glycans, O-glycans, and glycosphingolipids, and they display a high level of diversity. This diversity arises from possible sialic acid modifications such as acetylation, methylation, hydroxylation, and sulfation in addition to different glycosidic linkage types that connect sialic acids to subsequent carbohydrate residues in the chain. Although α2,3 and α2,6 glycosidic linkages to galactose (Gal) or N-acetylgalactosamine (GalNAc) are the most common types found in these sialoglycan structures. To some degree, virus host range specificity can be determined by the glycosidic linkage type, as seen for example in influenza viruses.

It has been shown that human influenza A viruses bind preferentially to sialylated glycans with an α2,6 linkage between the terminal sialic acid and Gal. Avian viruses, on the other hand, have a preference for binding glycans with an α2,3 linkage at this position. The molecules of the invention contain such sialylated linkages and may therefore be bound by these types of viruses, as exemplified here with haemagglutinin from influenza A. Many other pathogens including bacteria, fungi, parasites also bind sialylated glycans to gain entry or cause disease and thus the molecules described herein are predicted to also have therapeutic benefit for these conditions.

These findings teach that the spatially separated sialylated Fc fragment proteins of the invention may therefore have therapeutic utility as blockers of pathogen host cell interactions. However, they may also be beneficial post-infection. Influenza is commonly associated with deadly bacterial pneumonias. Interestingly these bacteria (e.g. group B streptococci and Streptococcus pneumoniae) by nature of having exposed sialylated capsules can bind directly to parainfluenza viruses that mutually facilitate their entry and spread in the body (Tong et al, Front Cell Infect Microbiol. 2018 Aug. 17; 8:280. doi: 10.3389/fcimb.2018.00280. eCollection 2018). The proteins of the present invention that bind and/or block sialic acid receptors may therefore block this interaction and thus help the body contain the bacteria that contributes significantly to mortality when infected with influenza.

The blockers could work in two ways. As with flu, by binding molecules that have an affinity for α2,3 sialic acid or by binding human receptors thus blocking their access by sialylated pathogens e.g. the aforementioned bacteria.

The inventors have noted that the sugars at Asn221 and Asn563 are located approximately 60 Å apart, the same approximate distance found between the sialic-acid binding domains within two monomers of a hemagglutinin trimer.

The use of DNA scaffolds has shown that bivalent scaffolds that present synthetic non-mammalian cell derived sialyl-LacNac ligands in 20-100 distance are as effective, or often more effective, than larger scaffolds at inhibiting haemagglutination by virus (Bandlow et al. JACS 139:16389-16397, 2017). Such scaffolds showed that 1360-fold enhancements over monovalent binding can be achieved with only two sugar ligands, provided the sugars are arranged at the 52-59 distance necessary to bind two of the three binding sites within a HA trimer simultaneously (Bandlow et al. Chembiochem. 20(2):159-165, 2019). Although DNA-based scaffolds allowed a defined arrangement of the sialyl-LAcNac ligands to be determined, DNA-based materials are more rigid than the Fc and are not facile to manufacture or use in vivo. These same studies also exposed inherent limitations of other spacers including PEG-based spacers (despite similar end-to-end length). The Fc molecules of the present invention will not suffer the same disadvantages as DNA scaffolds.

Without wishing to be bound by theory, the sugar moieties exposed in these molecules may therefore be able to block the sialic acid binding domains thus preventing access to sialic acid dependent viruses such as Flu and Newcastle disease virus, or other sialic acid dependent pathogens. As noted above, they may also block the interaction between pneumonia-associated bacteria and parainfluenza viruses, thus impeding the bacteria from gaining entry to the cell.

Examples of sialic-acid dependent pathogens that mediate disease or infection that these molecules can be used to treat or prevent include gram-positive bacteria such as Streptococcus pneumoniae, Streptococcus suis, Streptococcus agalactiae, Neisseria meningitidis, E. coli K1 and Campylobacter jejuni; or gram-negative bacteria such as Pseudomonas aeruginosa, Haemophilus influenzae, Haemophilus ducrey, Helicobacter pylori, Legionella pneumophila, Pasteurella multocida, Salmonella enterica and Vibrio cholerae; other bacteria such as Neospora caninum; parasites such as Plasmodium falciparum, Trypanosoma cruzi, Toxoplasma gondii and Leishmania donovani; and viruses such as influenza, Porcine Reproductive and Respiratory Syndrome Virus, rotavirus, Newcastle disease virus, coronaviruses (including Mers and Sars) and HIV-1(⁸²see Table 1) and viruses listed below. Roy and Mandal⁸⁶ also report that Leishmania donovani utilize sialic acids for binding and phagocytosis in the macrophages through selective utilization of siglecs and Impair the Innate Immune Arm.

See also: Matrosovich et al., Top Curr Chem 367:1-28, 2015; Mikulak et al. Frontiers in Immunology. Vol. 8: article 314, 2017, doi: 10.3389/fimmu.2017.00314; Wasik et al. Trends in Immunology. 24(12):991-1001, 2016; Dietrich et al., Protein Science. 26:2342-2354, 2017; Tong et al., Front Cell Infect Microbiol. 2018 Aug. 17; 8:280. doi: 10.3389/fcimb.2018.00280. eCollection 2018; Tong et al., Cell Microbiol. 2018 April; 20(4). doi: 10.1111/cmi.12818. Epub 2018 Jan. 16; Khatua et al. Indian J Med res. 138:648-662, 2013; and, Varki. Trends in Mol Med 14(8):351-360, 2008.

Suitable viruses include: Orthomyxoviridae e.g. influenza viruses, Coronaviridae e.g. bovine coronavirus, Paramyxoviridae e.g. Newcastle disease virus and Rubulaviruses (mumps), Caliciviridae e.g. norovirus, Picornaviridae e.g. human enterovirus, Reoviridae e.g. rotaviruses, Polyomaviridae e.g. Merkel cell polyomavirus which can cause human skin cancer, and Adenoviridae e.g. Ad37 which causes keratoconjunctivitis in human eye.

In a particular embodiment, the sialic-acid dependent pathogens that mediate disease or infection that the proteins of the invention, or a pharmaceutical composition comprising said protein, can be used to treat or prevent include: Streptococcus pneumoniae, Streptococcus suis, Streptococcus agalactiae, Neisseria meningitidis, E. coli, Campylobacter jejuni, Pseudomonas aeruginosa, Haemophilus influenzae, Haemophilus ducrey, Helicobacter pylori, Legionella pneumophila, Pasteurella multocida, Salmonella enterica, Vibrio cholerae, Neospora caninum, Plasmodium falciparum, Trypanosoma cruzi, Toxoplasma gondii, Leishmania donovani, influenza, Porcine Reproductive and Respiratory Syndrome Virus, rotavirus, Paramyxovirus (such as Newcastle disease virus and rubulavirus), norovirus, enterovirus, rotavirus, polyomavirus (such as e.g. Merkel cell polyomavirus), coronaviruses (including Mers and Sars), adenovirus (such as Ad37) and lentivirus (such as HIV-1).

According to a sixth aspect of the invention there is provided a method of preventing or treating an autoimmune or inflammatory disease, the method comprising providing a therapeutically effective amount of protein in accordance with the first, second or third aspects of the invention or another protein of the invention to a subject in need of such prevention or treatment. The subject may be a human subject.

According to a seventh aspect of the invention there is provided a method of preventing or treating a disease mediated through (i) binding of sialic acid dependent receptors, and/or (ii) binding of Fc gamma receptors, the method comprising providing a therapeutically effective amount of protein in accordance with the first, second or third aspects of the invention or another protein of the invention to a subject in need of such prevention or treatment. Suitably the subject is human. In an embodiment, the sialic acid-dependent receptor may be selected from the group consisting of: SIGLEC-1, SIGLEC-2, SIGLEC-3, SIGLEC-4, CD23, dec-1, dec-2, DC-SIGN, CLEC-4A, CLEC-4D, MBL, MMR and DEC-205. In an embodiment, the Fc gamma receptor is selected from the group consisting of: FcγRI, FcγRIIA, FcγRIIB, FcγRIIIA and FcγRIIIB. In an embodiment, the protein of the invention is capable of binding FcγRIIIA and C1q.

The disease may be an infection, a cancer or an autoimmune disease. The disease may be a flu or retroviral infection.

Molecules that comprise N563A substitution (e.g. N563A/C575A or C309L/N563A/C575A) and form multimers may be suitable for the treatment of cancer, particularly if introduced into a mAb or other antigen binding scaffold

Other suitable molecules are those with Asn at position 221, e.g. D221N/C309L/N297A/C575A and D221N/C575A. As these two mutants do not bind FcγRs they may be particularly suitable for preventing infection by, for example, flu as they would block binding without interfering with the neutralising IgGs that clear virus by FcγRIIIA dependent ADCC.

The medical uses or methods of treatment of the fifth, sixth or seventh aspects of the invention may employ the proteins of the invention in intravenous immunoglobulin (IVIG) or subcutaneous immunoglobulin (SCIG) therapy or intranasal or aerosol delivery for treatment or prevention of flu or other infection.

According to an eighth aspect of the invention there is provided a nucleic acid encoding a protein in accordance with the first, second or third aspects of the invention or another protein of the invention.

According to a ninth aspect of the invention there is provided a method of producing a protein in accordance with the first, second or third aspects of the invention or another protein of the invention, the method comprising expressing a nucleic acid in accordance with the sixth aspect of the invention in a host cell.

In a particular embodiment the host cell is a Chinese hamster ovary (CHO) cell.

### DETAILED DESCRIPTION OF THE INVENTION

The present invention is based upon the inventors' surprising finding that removal of the ability of the IgG heavy chain constant region and the tailpiece region to covalently bond to another polypeptide via disulphide bridges (other than via the hinge region that are capable of forming disulphide bridges with the companion polypeptide), in particular the loss of the cysteine in the IgM tailpiece, leads to an increase in ability of the chimeric protein to bind to type I and II receptors, particularly if a polypeptide chain of the chimeric protein has also been mutated to introduce a glycosylation site at or close to the amino terminus (e.g. D221N mutation). This has important therapeutic implication for their use, e.g. as IVIG, SCIG and in vaccines. Furthermore, the alterations made to the primary sequence allows the protein to be monomeric in physiological conditions. The cysteine residue proteins of the invention, as described herein, exhibit enhanced binding to low-affinity inhibitory Fcγ- and glycan-receptors that may be useful in the treatment of or vaccination against disease.

The ability of a molecule to bind to glycan receptors, in particular Dendritic Cell-Specific Intercellular adhesion molecule-3-Grabbing Non-integrin (DC-SIGN), is associated with therapeutic utility in IVIG or SCIG. Previously it has been found that less than 5% of native, monomeric IgG molecules are correctly glycosylated in a way that allows them to interact with DC-SIGN, thus rendering these molecules unsuitable for therapeutic purposes.

The Fc receptor-binding molecules of the invention have increased affinity for various sialic acid dependent receptors such as sig-1, sig-2, sig-3, sig-4 and CD23, which is advantageous in terms of their ability to bind to such receptors. Many of the oligomeric or polymeric Fc receptor-binding molecules previously described in the prior art have been shown to activate the complement cascade. This is a significant disadvantage in a potential therapeutic molecule, due to the risk of adverse consequences, such as anaphylactic shock. Surprisingly, certain of the proteins of the invention do not lead to the activation of the complement cascade (see Table 1).

Surprisingly, particular molecules of the invention (e.g. glycosylation adapted proteins) exhibit significant binding to C1q and FcγRIIIA. Binding to FcγRIIIA or C1q is important for activating antibody-dependent cell-mediated cytotoxicity (ADCC) in human NK cells. The glycosylation adapted proteins of the invention can accommodate one or more payloads. In embodiments where the payload is capable of binding to a cancer antigen, e.g. if the payload is an immunoglobulin binding protein (such as Fab, F(ab′)2, scFv or nanobody etc.) or a non-immunoglobulin scaffold protein (such as disclosed in Skriec et al., Trends Biotechnol. 2015 July; 33(7):408-18. doi: 10.1016/j.tibtech.2015.03.012. Epub 2015 Apr. 27), such molecules would be particularly suitable for use in the treatment of cancer.

Surprisingly, particular IgG1-Fc mutants containing complex sialylated glycans attached to the N-terminal Asn221 sequon bound influenza A and B virus hemagglutinin and disrupted influenza A-mediated agglutination of human erythrocytes (Blundell et al, 2019. Journal of Immunology 202(5):1595-1611). These molecules are particularly suited for use as agents to block pathogen infectivity, such as viruses or bacteria that utilise sialic acid dependent receptors to gain cellular entry, e.g., influenza and Newcastle disease viruses. Particularly suitable molecules in this context are D221N/C309L/N297A/C575A and D221N/C575A (see Table 3). Surprisingly, the molecules that bound haemagglutinin had sugars at each end and form as monomers, whereas those that multimerised did not bind haemagglutinin strongly.

According to a first aspect of the invention there is provided a chimeric Fc receptor binding protein comprising two chimeric polypeptide chains; wherein each chimeric polypeptide chain comprises a hinge region, an immunoglobulin G heavy chain constant region and an immunoglobulin tailpiece region, wherein the amino acid sequence of each polypeptide chain is altered as compared to the native sequences from which the immunoglobulin G heavy chain constant region and the immunoglobulin tailpiece region are derived to remove cysteine residues that form extrinsic disulphide bonds, and wherein each polypeptide chain comprises at least one glycosylation site.

Suitably, each polypeptide of the invention comprises two immunoglobulin G heavy chain constant domains.

Suitably, each polypeptide of the invention comprises at least one cysteine residue in the hinge region that can form an inter-chain disulphide bridge with the corresponding cysteine on the companion polypeptide. Suitable, the hinge region comprises 2, 3, 4 or more cysteine residues that can form inter-chain disulphide bridges with cysteine residues in the companion polypeptide.

In one embodiment, each immunoglobulin G heavy chain constant region comprises two immunoglobulin domains CH2 and CH3 (also known as Cγ2 and Cγ3). The IgG heavy chain constant region may be selected from the group consisting of IgG1, IgG2, IgG3 and IgG4. Suitably, the IgG heavy chain constant region is IgG1 or IgG2.

The chimeric proteins of the invention may comprise residues 221 to 447 of human IgG1 combined with residues 561 to 576 of the tailpiece of human IgM. Residues 221 to 230 of human IgG1 represents the hinge region.

Certain of the chimeric proteins of the invention may form monomers.

Proteins of the invention feature adaptation of the amino acid sequence as compared to the sequence found in native/wild-type immunoglobulins from which they are derived. These may inhibit polymerisation of the chimeric/hybrid proteins of the invention and yield enhanced receptor binding ability.

Suitably the adaptation of the amino acid sequence is the loss of cysteine residues that are involved in extrinsic disulphide bridge formation within the IgG heavy chain constant region and the tailpiece region. Suitably, as compared to the native sequence from which the region was obtained, the adaptation is the loss of a cysteine residue within the tail region, such as at a position corresponding to position 248 of SEQ ID NO: 1. Furthermore, with regard to the known Hexa-Fc molecules there is also the loss of the cysteine in the IgG constant heavy region corresponding to position 89 of SEQ ID NO: 1.

The loss of a cysteine can be by their substitution or deletion or chemical modification to disrupt the ability to form disulphide bonds. In one embodiment, the tailpiece cysteine residue is modified using a thiol capping agent; a compound which reacts with sulphydryl groups in reduced cysteine residues, preventing them from forming disulphide bonds. Examples of suitable thiol capping agents include iodoacetic acid, iodoacetamide and N-ethylmaleimide.

In a suitable embodiment, a cysteine residue, such as the cysteine residue corresponding to residue 248 of SEQ ID NO:1, is replaced with a different amino acid. The cysteine residue may be substituted by any amino acid residue (for example an alanine, aspartic acid, glutamic acid, phenylalanine, glycine, histidine, isoleucine, lysine, leucine, methionine, asparagine, proline, glutamine, arginine, serine, threonine, valine, tryptophan or tyrosine residue). Suitably, the cysteine residue corresponding to residue 248 of SEQ ID NO:1, is replaced with an alanine residue. Suitably, and with regard to Hexa-Fc molecules the cysteine residue corresponding to residue 89 of SEQ ID NO:1, is replaced with a leucine residue.

An example of a protein of the invention comprising such a modification comprises a polypeptide chain with the sequence disclosed in SEQ ID NO: 2. The protein of SEQ ID NO: 2 is a particularly useful example of a protein of the invention. It represents an embodiment that is highly suited to medical uses and methods in which it is desired for proteins of the invention to bind to sialic acid dependent receptors (for example to prevent or treat diseases mediated through binding of sialic acid dependent receptors).

Various aspects and embodiments of the invention will now be further described in the following paragraphs.

### Glycosylation Site Modifications

The chimeric polypeptides of the invention may have zero, one, two, three or more glycosylation sites. Suitably, the “cysteine residue molecules” that form monomers will have at least one glycosylation site.

In one embodiment, there is a glycosylation site at a position that corresponds to residue 77 of SEQ ID NO: 1. In another embodiment, there is a glycosylation site at a position that corresponds to residue 236 of SEQ ID NO: 1.

The inventors have found that the introduction of an artificial glycosylation site is able to give rise to a protein with greater sialylation than a protein without such an artificial glycosylation site, and thus may yield a protein with greater efficacy for use in sialic acid dependent or other sialic acid independent therapies. Such sialylation may also impart longer half-lives to said molecules.

It is known that molecules rich in mannose or those that bind DC-SIGN, DEC-205, MMR and MBL may be more suitable for vaccine approaches. All these receptors are found on macrophages which are antigen presenting cells and have been implicated in vaccine efficacy e.g. N-glycosylation converts non-glycoproteins into mannose receptor ligands and reveals antigen-specific T cell responses in vivo. (Kreer C, Kuepper J M, Zehner M, Quast T, Kolanus W, Schumak B, Burgdorf S. Oncotarget. 2017 Jan. 24; 8(4):6857-6872. doi: 10.18632/oncotarget.14314. Thus, proteins of the invention that bind these receptors are predicted to be useful for use in or as vaccines.

Thus, in one embodiment, the proteins of the first or second aspects of the invention incorporate an adaptation that provides an additional glycosylation site. Suitably, the adaptation provides an additional glycosylation site in the hinge region, such as by gain of an asparagine residue, such as at a position corresponding to position 1 in SEQ ID NO: 1 (corresponding to D221 of human IgG). Suitable, the adaptation to add an asparagine residue at position 1 is by substitution of the native aspartic acid residue.

In another embodiment, there is a glycosylation site at a position that corresponds to residue 1 of SEQ ID NO:1. In a particular embodiment, the polypeptide chain comprises three glycosylation sites located at position 1, 77 and 236 corresponding to the position in SEQ ID NO: 1.

An example of a protein of the invention comprising such a modification comprises a polypeptide chain with the sequence disclosed in SEQ ID NO: 3.

In one embodiment, each chimeric polypeptide chain in a proteins of the first or second aspects of the invention is altered as compared to the native sequences from which the hinge region, immunoglobulin G heavy chain constant region and the immunoglobulin tailpiece region are derived to possess one additional glycosylation site. In a particular embodiment, each polypeptide chain comprises an additional glycosylation site in the hinge region as compared to the native sequence from which the hinge region is derived. In a particular embodiment, the additional glycosylation site (in the chimeric polypeptide chain) is at a position that corresponds to residue 1 of SEQ ID NO:1.

The presence of an additional glycan at the artificial glycosylation site may also confer a further advantage as it may increase the protein's stability. Currently, in order to increase immunoglobulin stability, immunoglobulins are often chemically glycosylated, for example by in vitro enzymatic or non-enzymatic reactions. However, the presence of an artificial glycosylation site allows such modifications to be introduced by cells expressing the proteins, and thus may eliminate the need for this additional step of chemical glycosylation. As a result, the proteins of the invention may be produced in a more cost and time effective manner than traditional agents used in IVIG treatment.

The adaptation to remove extrinsic disulphide bond forming cysteine residues from the native immunoglobulin regions that the polypeptide is derived from (other than the hinge region) and to add (such as via addition or substitution) one or more additional glycosylation site such as an asparagine residue that is capable of being glycosylated, reduces the polymerisation of the chimeric protein, such that most chimeric proteins adopt the monomeric form, and yields molecules with enhanced receptor binding ability.

In a suitable embodiment, a protein of the invention may also comprise an adaptation to a glycosylation site of the immunoglobulin G constant heavy region (such as the immunoglobulin G heavy chain constant region Cγ2). Such a glycosylation site may be found, for example, at a residue corresponding to N77 of SEQ ID NO: 1 (corresponds to N297 in IgG Cγ2). In a particular embodiment, the asparagine residue at a residue corresponding to N77 of SEQ ID NO: 1 is substituted by another amino acid, such as alanine. In another suitable embodiment, a protein of the invention may also comprise an adaptation to a glycosylation site of the tail region (such as the IgM tail region). Such a glycosylation site may be found, for example, at a residue corresponding to N236 of SEQ ID NO: 1 (corresponds to N563 of IgM). In a particular embodiment, the asparagine residue at a residue corresponding to N236 of SEQ ID NO: 1 is substituted by another amino acid, such as alanine. Proteins of the invention incorporating such adaptations may be of particular utility in application requiring and making use of the ability to bind sialic acid-dependent receptors. Examples of proteins of the invention comprising such a modification comprise a polypeptide chain with the sequence disclosed in any of: SEQ ID NOs: 4-8.

WO2016/009232 (Liverpool School of Tropical Medicine) discloses multimeric forms of chimeric proteins that have alterations/adaptations in the number of glycosylation sites. The proteins of the invention may incorporate any of the glycosylation adaptations disclosed therein.

In one embodiment, the amino acid sequence of each polypeptide in a protein of the first, second or third aspects of the invention is altered as compared to the native sequences from which the hinge region, immunoglobulin G heavy chain constant region and the immunoglobulin tailpiece region are derived to possess one fewer glycosylation site. In a particular embodiment, as compared to the native immunoglobulin sequence there is a loss of the asparagine residue corresponding to residue 77 of SEQ ID NO:1. In another embodiment, as compared to the native immunoglobulin sequence there is a loss of the asparagine residue corresponding to residue 236 of SEQ ID NO:1.

In one embodiment, the amino acid sequence of each polypeptide is altered as compared to the native sequences from which the hinge region, immunoglobulin G heavy chain constant region and the immunoglobulin tailpiece region are derived to possess two fewer glycosylation sites. In a particular embodiment, as compared to the native immunoglobulin sequences there is a loss of the asparagine residue corresponding to residue 77 of SEQ ID NO:1 and residue 236 of SEQ ID NO:1.

Without wishing to be bound by any hypothesis, the inventors believe that the combination of loss of key cysteine residues that would otherwise be able to form extrinsic disulphide bridges between protein monomers or individual polypeptides that form the monomer, in combination with the capacity for larger glycans to be added (or removed) at the glycosylation sites present within the monomers, significantly inhibits polymerisation of the proteins of the invention. As discussed elsewhere in the invention, these changes are sufficient to decrease the proportion of the certain proteins occurring in polymeric form from greater than 80% to less than <1%.

A further advantage of the modification of glycosylation observed is that, the larger and more complex glycans present are more likely to terminate in sialic acid (neuraminic acid). Glycans terminating in this manner are known to interact with DC-SIGN, and enhanced binding to DC-SIGN and SIGLEC-1 is observed in respect of proteins of the invention.

### Tailpiece Modifications

IgM and IgA heavy chain constant regions contain a ‘tailpiece’ of 18 amino acids that contains a cysteine residue essential for polymerization. An alternative tailpiece region is the tailpiece region of human IgE variant (termed IgE tailpiece). IgE-tailpiece (IgE-tp), differs from classical IgE (IgE-c) in possessing an eight-amino acid carboxy-terminal tailpiece that terminates in a cysteine residue (ESSRRGGC; SEQ ID NO: 45) [Quinn et al. Sci. Rep. 6, 20509; doi: 10.1038/srep20509; 2016].

In various embodiments, the tailpiece region is based upon the tailpiece region of an immunoglobulin selected from the group consisting of: IgM, IgA, and IgE. In a particular embodiment, the tailpiece region is based on the tailpiece region from human IgM, which is PTLYNVSLVMSDTAGTCY (SEQ ID NO: 14) (Rabbitts et al. Nucl. Acid Res. 9(18):4509-4524, 1981; Smith et al. J. Immunol. 154:2226-2236, 1995). Further suitable variants of the tailpiece region of IgM are described in Sorensen et al. (J. Immunol. 156:2858-2865, 1996). An alternative tailpiece region is the tailpiece region of human IgA, which is PTHVNVSVVMAQVDGTCY (Putnam et al. J. Biol. Chem. 254:2885-2674; SEQ ID NO: 15). An alternative tailpiece region is the tailpiece region of human IgE-tp, which is (ESSRRGGC; SEQ ID NO: 45). Alternatively, the tailpiece region may be a synthetic polypeptide. By which we mean the sequence may not exist within or be derived from a natural immunoglobulin tailpiece. In this circumstance, the synthetic immunoglobulin tailpiece sequence is between 12 and 26 amino acids in length and serves as a spacer or scaffold sequence that may or may not comprise one or more glycosylation sites. A synthetic immunoglobulin tailpiece may be suitable for use in the haemagglutinin binding proteins of the invention as the location of the carboxy-terminal sugar (glycan) can be optimally positioned so that it is approximately 60 Å from the sugar at the amino terminus.

The tailpiece region in the proteins of the invention can be adapted based on the native sequences to lack the cysteine residues that can form extrinsic disulphide bridges. The tailpiece may be attached via a spacer region.

### Adaptation of Tailpiece Amino Acid Sequence to Inhibit Polymerisation

The adaptation to remove extrinsic disulphide bond forming cysteine residues from the native tailpiece region that the polypeptide is derived from, or exclude them in a synthetic tailpiece, reduces the polymerisation of the chimeric protein, such that most chimeric proteins adopt the monomeric form, and yield molecules with enhanced receptor binding ability.

As noted above, immunoglobulin tailpieces for incorporation in the proteins of the invention may be based upon any immunoglobulin molecule. Suitably a tailpiece may be based upon the tailpiece of an immunoglobulin selected from the group consisting of: IgM, IgA, and IgE.

A tailpiece based upon that of IgM is particularly suitable for incorporation in the proteins of the invention. Exemplary adaptations are described herein with reference to the IgM tailpiece (which is incorporated in the reference protein of SEQ ID NO: 1, and the exemplary proteins of the invention of SEQ ID NO: 2 to SEQ ID NO:11). It will be appreciated that tailpieces of other immunoglobulins may be adapted at residues corresponding to those exemplified in respect of IgM. Furthermore, tailpieces derived from immunoglobulins other than IgM may be adapted in the same manner as described in respect of IgM.

Tailpieces suitable for incorporation in the proteins of the invention may, as long as they comprise relevant adaptations, share at least 55% identity with a native immunoglobulin tailpiece, such as the IgM tailpiece. Indeed, a suitable tailpiece, as long as suitably adapted, may share at least 55%, at least 65%, at least 75%, at least 80%, at least 85%, at least 90%, at least 95% or more identity with the sequence of a corresponding portion of a native immunoglobulin tailpiece.

In particular, tailpieces suitable for incorporation in the proteins of the invention may share at least 55% identity with the IgM-derived sequences of SEQ ID NO: 2 (i.e. amino acid residues 232-249 of SEQ ID NO: 2). Suitably, a tailpiece for incorporation in a protein of the invention may share at least 55%, at least 65%, at least 75%, at least 80%, at least 85%, at least 90%, at least 95% or more identity with residues 232-249 of SEQ ID NO:2.

Proteins of the invention may include at least one glycosylation site within the tailpiece. These may be naturally occurring glycosylation sites retained from the native immunoglobulin tailpiece sequence. Alternatively, the proteins of the invention may include artificially introduced glycosylation sites in the tailpiece region, or combinations of naturally occurring and artificial sites. Suitably, an additional glycosylation site is introduced at the carboxy terminus of the tailpiece.

Molecules with enhanced glycosylation may possess an enhanced in vivo half-life.

Bas et al., (Immunol. 2019 Jan. 25. pii: ji1800896. doi: 10.4049/jimmunol.1800896) have shown that sialylation of the Fc improves the in vivo half-life of the Fc up to nine-fold (from days to weeks), and thus by nature of being hypersialylated the inventors anticipate that the proteins of the invention will also have a favourable pharmaco-kinetic profile in vivo. A long in vivo half-life is therapeutically important especially for flu, and is a significant drawback to neuraminidase inhibitors, including Oseltamivir whose in vivo half-life is only 6-10 hours.

### Spacer Region

As touched upon above, the protein of the invention may comprise a spacer region. Suitably, the spacer region may be between the Fc receptor binding portion and the tailpiece region.

A suitable spacer region may be at least one, at least two, at least three, at least four, at least five, at least six, at least seven, at least eight, at least nine, at least ten, or more amino acid residues long. More suitably, the spacer region may be four amino acid residues long. In the exemplary protein of the invention as set out by SEQ ID NO: 2, the spacer region may be found at residues 228 to 231.

Suitably the spacer region may have the sequence LVLG (SEQ ID NO: 16).

The presence of a spacer region may improve the disposition of the tailpiece carbohydrate (for example corresponding to amino acid residue N236 of the protein if the invention as set out in SEQ ID NO:1 or SEQ ID NO:2) and thus improve interactions with glycan receptors without compromising interactions mediated through the Fc constant domains.

### Hinge Region Modifications

In addition to the Fc receptor binding portion and the tailpiece region, the polypeptide chains that form the proteins of the invention further comprise a hinge region.

In a suitable embodiment, the hinge region may be located at the N-terminus of the Fc receptor binding portion. The hinge region may be a natural or synthetic hinge region. Suitably, the hinge region comprises at least one cysteine residue which is capable of forming a disulphide bridge with the corresponding cysteine on the other polypeptide so as to keep the two polypeptides together to form the Fc monomer. Hydrophobic amino acids in the tailpiece (e.g. valines) are also likely to allow the two polypeptides to interact/cluster together through non-covalent interactions.

In a suitable embodiment, the hinge region is all or part of a natural hinge region. A natural hinge region is one that is naturally found between the Fc and Fab portion of an antibody. A natural hinge region may be derived from the same species as the Fc receptor binding portion. Alternatively, it may be derived from a different species. Natural hinge regions typically have three main sections, an upper, middle and lower section. The upper section determines the arrangement between the two Fab regions and mediates flexibility and orientation of each Fab arm. Two cysteine residues (e.g. Cys²²⁶ and Cys²²⁹ in IgG) in the middle region form inter-chain disulphide bonds between the two heavy chains to join these together. The lower hinge is responsible for the flexibility and positioning of the Fc region relative to the Fab arms. The complete hinge region in IgG1 between the Fab and Fc fragments is formally composed of 23 residues (EPKSCDKTHTCPPCPAPELLGGP-SEQ ID NO: 17) between Val²¹⁵ and Ser²³⁹ in which the Fab region formally ends at Val²¹⁵ and the Fc region starts at Ser²³⁹. In IgG1, the lower hinge runs from residue 216 to 226, the middle from 227 to 230 and the lower from 231 to 238. There is no need to use the full “formal” length of a natural hinge in the proteins of the invention. A suitable hinge region for use in the present invention is DKTHTCPPCP (SEQ ID NO: 18). Which runs from Asp²²¹ to Pro²³⁰. There are two cysteine residues in this region that form disulphide bonds with cysteines in the companion polypeptide and thereby assist in keeping the two polypeptide chains together to form the Fc monomer.

Within native immunoglobulins, the two heavy chains are connected in the hinge region by a variable number of disulphide bonds: 2 for IgG1 and IgG₄, 4 for IgG₂ and 11 for IgG₃.

In a suitable embodiment, a natural hinge region (or fragment thereof) may be derived from an antibody of the same class or subclass as the Fc receptor binding portion. Alternatively, it may be derived from an antibody of a different class or subclass as the Fc receptor binding portion.

In a suitable embodiment, the hinge region is derived from IgG1. More suitably, the hinge region may be derived from human IgG1. By way of example, the protein of the invention according to SEQ ID NO:2, comprises a hinge region derived from human IgG1.

It will be appreciated that the hinge region may serve as the location for the insertion and/or attachment of one or more additional N-linked glycosylation site(s) in the protein of the invention.

In a suitable embodiment, the N-terminus of the hinge region may be glycosylated in a way so as to inhibit polymerisation of the protein of the present invention. Suitably, the glycosylation may be at a position corresponding to residue 1 of SEQ ID NO:1. Glycosylation of the hinge region may be beneficial as it may result in an exposed glycan, which may modify the function of the protein of the invention. Examples of proteins of the invention comprising such a modification comprise a polypeptide chain with the sequence disclosed in any of: SEQ ID NOs: 3, 5, 6, 7, 9 and 59. By way of example, and as further explained in the Examples section of this description, glycosylation of the hinge region may reduce the protein's interactions with Fc-gamma receptors, while increasing interactions with sialic acid dependent receptors such as SIGLEC-1.

In another embodiment, the hinge region is synthetic. A synthetic hinge region is one that differs in length or sequence from a hinge region which is found naturally. By way of example, the difference in length between a synthetic and natural hinge region may be as a result of the addition or deletion of residues in the synthetic hinge region (for example addition or deletion of cysteine residues). A difference in sequence between a synthetic and natural hinge region may be as a result of a substitution of one or more residues in the synthetic hinge region (for example substitution of a cysteine residue with another residue such as serine or alanine).

In a suitable embodiment, a hinge region may be at least one, at least two, at least three, at least four, at least five, at least six, at least seven, at least eight, at least nine, at least ten, at least fifteen, at least twenty, at least 23, at least twenty-five, at least thirty, or more amino acid residues long.

By way of example and not limitation, a protein of the invention may comprise a hinge region, wherein the hinge region has a sequence selected from the group consisting of: VPSTPPTPSPSTPPTPSPS (SEQ ID NO: 19), VPPPPP (SEQ ID NO: 20), EPKSCDKTHTCPPCP (SEQ ID NO: 21), ERKCCVECPPCP (SEQ ID NO: 22), ESKYGPPCPSCP (SEQ ID NO: 23), CPPC (SEQ ID NO: 24), CPSC (SEQ ID NO: 25), and SPPC (SEQ ID NO: 26). Other suitable natural and synthetic hinges will be known to those skilled in the art. The two cysteines found in, e.g.: DKTHTCPPCP (SEQ ID NO: 27) are used for interdomain disulphide bond formation pairing CH2 and CH3 of IgG1.

In a suitable embodiment, a hinge region may be multiple copies of a natural hinge in tandem, e.g. 2, 3, 4, 5, 6 or more copies of a natural hinge sequence linked together. Increasing the length of the hinge will serve to distance the payload from the immunoglobulin regions.

### Adaptation of Hinge Region Amino Acid Sequence to Inhibit Polymerisation

Hinge regions for incorporation in the proteins of the invention may be based upon any immunoglobulin molecule. Suitably a hinge region be based upon the hinge region of an immunoglobulin selected from the group consisting of: IgG, IgA, IgE, IgD and IgM. More suitably, the hinge region of an IgG immunoglobulin may be selected from the group consisting of IgG1, IgG2, IgG3 and IgG4.

A hinge region based upon that of immunoglobulin IgG1 is particularly suitable for incorporation in the proteins of the invention. Exemplary adaptations of a hinge region are described herein with reference to the IgG1 hinge region (which is incorporated in the reference protein of SEQ ID NO: 1, and the exemplary proteins of the invention of SEQ ID NO: 2 to SEQ ID NO: 11).

It will be appreciated that hinge regions of other IgG immunoglobulins may be adapted at residues corresponding to those exemplified in respect of IgG1. Furthermore, hinge regions derived from immunoglobulins other than IgG may be adapted in the same manner as described in respect of IgG1.

Hinge regions suitable for incorporation in the proteins of the invention may, as long as they comprise relevant adaptations, share at least 55% identity with a native immunoglobulin hinge region, such as the IgG hinge region. Indeed, a suitable hinge region, as long as suitably adapted, may share at least 55%, at least 65%, at least 75%, at least 80%, at least 85%, at least 90%, at least 95% or more identity with the sequence of a corresponding portion of a native hinge region.

In particular, hinge regions suitable for incorporation in the proteins of the invention may, as long as they comprise the adaptations found in SEQ ID NO:2, share at least 55% identity with the IgG hinge region derived sequences of SEQ ID NO: 2. Suitably a hinge region for incorporation in a protein of the invention may share at least 55%, at least 65%, at least 75%, at least 80%, at least 85%, at least 90%, at least 95% or more identity with the residues of the hinge region of SEQ ID NO:2.

### Adaptation of Hinge Region Glycosylation to Inhibit Polymerisation

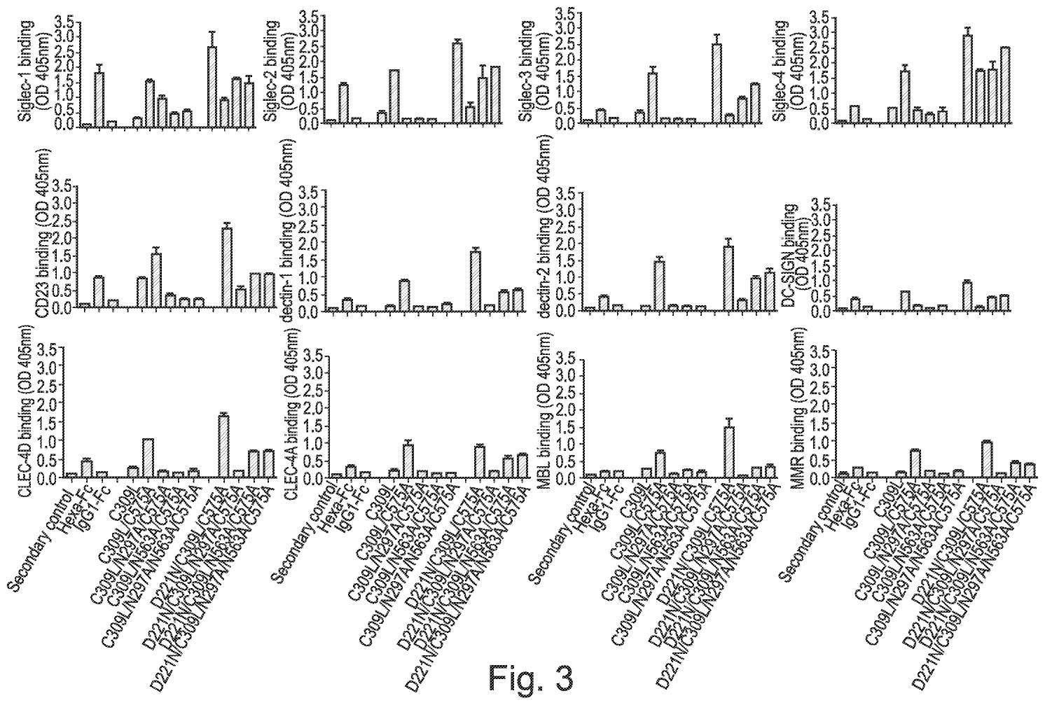

Proteins of the invention may include a glycosylation site within the hinge region. The glycosylation site may be a naturally occurring glycosylation site retained from a native immunoglobulin hinge region sequence. Alternatively, the proteins of the invention may include an artificially introduced glycosylation site in the hinge region, or combinations of naturally occurring and artificial sites. In one embodiment, the hinge region of the chimeric polypeptide sequence is adapted to introduce a glycosylation site that is not present in the natural sequence from which the region was derived. In one embodiment, the hinge region in the chimeric polypeptide of the invention comprises an asparagine (N) residue at position 1 (corresponding to the position in SEQ ID NO: 1), whereas in the native sequence it is aspartic acid (D). The presence of this additional glycosylation site enhances the glycan receptor binding property of the chimeric protein. For example, compare D221N/C309L/N297A/N563A/C575A against C309L/N297A/N563A/C575A (see FIG. 3).

### Payloads

As mentioned elsewhere in this specification, the protein of the invention, particularly those according to the third aspect of the invention, may be conjugated to a therapeutic payload. The term “therapeutic payload” as used herein refers to a compound which itself has a therapeutic effect. The therapeutic effect of a therapeutic payload may be in addition to, or independent of, the therapeutic effect of the protein of the invention.

The therapeutic payload could be any moiety with a therapeutic effect. For example, a chemical (e.g. toxin), an antigenic peptide or an “antigen-binding domain”, for example, an antibody-derived domain capable of binding to an antigen. The antigen-binding domain may comprise an antibody light chain variable region (VL) and an antibody heavy chain variable region (VH): however, it need not comprise both. For example, Fd fragments have two VH regions and often retain some antigen-binding ability. Examples of “antigen-binding domains” (or fragments of an antibody) that can be attached to the hinge region (as a payload) include a: Fab, F(ab′)2, Fd, Fv, scFV, dAb and nanobody.

The antigen binding region could be any immunoglobulin fragment such as Fab, FAb2 or scFV. Suitable proteins of the invention could be IgG antibodies with a tailpiece region attached at the carboxy terminus.

In a suitable embodiment, the proteins of the present invention, particularly those according to the third aspect of the invention, or one or both individual polypeptide chains that form such proteins of the invention may be conjugated to a therapeutic payload. Suitably the payload may be conjugated to the Fc receptor binding portion of the protein of the invention. Alternatively, it may be conjugated to the hinge region of the protein of the invention. The payload may be chemically conjugated to the Fc receptor binding portion of the protein of the invention or it may be part of the DNA sequence that encodes a protein of the invention such that it is conjugated by usual amino acid linkage. Suitably, the payload is an immunoglobulin region (such as Fab, F(ab′)2, scFV or nanobody), an antigenic peptide or a chemically synthesised glycan.

In embodiments where the protein of the invention, particularly those according to the third aspect of the invention, is conjugated to a therapeutic payload, such payload may be conjugated to the hinge region of one or both polypeptide chains. It will be appreciated that the hinge region can be designed to adjust the distance between the Fc receptor binding portion and the therapeutic payload, if present. When the therapeutic payload is conjugated to the protein of the invention, increased distance between the Fc receptor binding portion and the therapeutic payload may be desirable in order to provide sufficient space for the attachment of a glycan molecule to a glycosylation site. Suitably, the payload can be conjugated to the hinge region by chemical conjugation, or it could be incorporated as part of the DNA encoding the protein of the invention. Suitably, the hinge region may also provide space for the attachment of a glycan molecule to an artificial glycosylation site (for example at a residue corresponding to residue 1 of SEQ ID NO: 1, as found in SEQ ID NO: 3).

### Exemplary Proteins of the Invention

Examples of monomeric proteins of the invention that lack cysteines within the IgG HC and tailpiece regions capable of forming extrinsic disulphide bonds are set out in SEQ ID NO: 2-8. A protein of the invention may comprise an amino acid sequence as disclosed in any of SEQ ID NO: 2 to SEQ ID NO: 8. In a suitable embodiment, a protein of the invention may consist of an amino acid sequence as disclosed in any of SEQ ID NO: 2 to SEQ ID NO: 8.

The specific adaptations, with respect to hexa-Fc molecule sequence (disclosed in SEQ ID NO: 1), present in each of the polypeptides that have the sequence depicted in SEQ ID Nos: 2-8, are shown in Table 4 and are apparent from the nomenclature of the molecule (e.g. C309L has a leucine residue substituted in place of cysteine at position 309 of the Hexa-Fc protein).

The chimeric polypeptide of SEQ ID NO: 1 comprises residues 221 to 447 of human IgG1 (corresponding to residues 1 to 227 of SEQ ID NO: 1) in combination with residues based upon, and adapted from, 558 to 576 of the tail-piece of human IgM (corresponding to residues 232 to 249 of SEQ ID NO: 1). SEQ ID NO: 1 is the sequence present in hexa-Fc. Relative to the wild-type immunoglobulin sequences from which they are derived this sequence has a cysteine residue in place of the native leucine at position 89 and a leucine in place of the histidine at position 90.

SEQ ID NO: 2 corresponds directly to SEQ ID NO: 1, save for the presence of a leucine residue at position 89, a histidine at position 90, and an alanine residue at position 248. This molecule is also referred to as C309L/C575A. The nomenclature does not refer to the His at position 90 as this is the amino acid found in wild-type IgG1.

SEQ ID NO: 3 corresponds directly to SEQ ID NO: 2, save for the presence of a D to N substitution at residue 1 of SEQ ID NO: 2. This molecule is also referred to as D221N/C309L/C575A. As discussed above, this substitution introduces a new glycosylation site in the protein of SEQ ID NO: 2.

SEQ ID NO: 4 corresponds directly to SEQ ID NO: 2, but wherein one of the glycosylation sites within SEQ ID NO: 1 has been mutated; in particular, with the N236A substitution. This molecule is also referred to as C309L/N563A/C575A.

SEQ ID NO: 5 corresponds directly to SEQ ID NO: 3, but wherein the two glycosylation sites within SEQ ID NO: 1 have been mutated; in particular, with the following two substitutions: N77A and N236A. This molecule is also referred to as D221N/N297A/C309L/N563A/C575A.

SEQ ID NO: 6 and 7 each correspond directly to SEQ ID NO: 3, but wherein one of the glycosylation sites within SEQ ID NO: 1 have been mutated; in particular, SEQ ID NO:6 comprises the N236A substitution (this molecule is also referred to as D221N/C309L/N563A/C575A); and SEQ ID NO: 7 comprises the N77A substitution (this molecule is also referred to as D221N/C309L/N297A/C575A).

SEQ ID NO: 8 correspond directly to SEQ ID NO: 2, but wherein one of the glycosylation sites within SEQ ID NO: 1 has been mutated; in particular, SEQ ID NO:8 comprises the N77A substitution (this molecule is also referred to as /C309L/N297A/C575A).

SEQ ID NO: 9 correspond directly to SEQ ID NO: 1, but comprises the D to N substitution at residue 1, N77A, a histidine at position 90, N236A substitution and C248A substitution (this molecule is also referred to as D221N/N297A/N563A/C575A).

SEQ ID NO: 9 correspond directly to SEQ ID NO: 9, but comprises the D at position 221. (N297A/N563A/C575A).

SEQ ID NO: 11 correspond directly to SEQ ID NO: 2, except for having an alanine residue at position 248. (C309L).

SEQ ID NO: 59 corresponds directly to SEQ ID NO: 1, save for the presence of a D to N substitution at residue 1 of SEQ ID NO: 1, a histidine at position 90, and an alanine residue at position 248 (this molecule is also referred to as D221N/C575A).

In a particular aspect of the invention there is provided a chimeric Fc receptor binding protein comprising two chimeric polypeptide chains; wherein each chimeric polypeptide chain comprises a hinge region, an immunoglobulin G heavy chain constant region and an immunoglobulin tailpiece region, wherein the amino acid sequence of each polypeptide chain is altered as compared to the native sequences from which the hinge region, immunoglobulin G heavy chain constant region and the immunoglobulin tailpiece region are derived to lack any cysteines from within the immunoglobulin G heavy chain constant region and immunoglobulin tailpiece region capable of forming extrinsic disulphide bonds and wherein the protein is one disclosed in any of Tables 1-4.

In a particular aspect of the invention there is provided a chimeric Fc receptor binding protein, wherein the protein is one disclosed in any of Tables 1-4.

In a particular aspect of the invention there is provided a chimeric Fc receptor binding protein comprising two chimeric polypeptide chains; wherein each chimeric polypeptide chain comprises an amino acid sequence as disclosed in any of SEQ ID Nos 1-11. In a suitable embodiment, the chimeric Fc receptor binding protein possess a therapeutic payload. In suitable embodiment, the therapeutic payload is attached to the hinge region, particularly at position 1 of the hinge region (corresponding to position 1 in SEQ ID NO: 1).

It will be appreciated that in the chimeric polypeptides, where the full IgG or IgM sequences are not present, numbering of residues based upon the full-length IgG or IgM molecules is no longer informative. Accordingly, we will also refer in this disclosure to a reference chimeric protein sequence, which is set out as SEQ ID NO: 1. This sequence represents a single chimeric polypeptide chain. When referring to this sequence, Cys575 of the full-length IgM sequence is renumbered as Cys248 of the chimeric polypeptide chain (the 248^{th} residue of SEQ ID NO: 1).

For the avoidance of doubt, a protein consisting of chimeric protein/polypeptide chains having the sequence set out in SEQ ID NO: 1 will not constitute a protein of the invention, since it will not incorporate the requisite adaptations.

In a particular aspect of the invention there is provided a chimeric Fc receptor binding protein comprising two chimeric polypeptide chains; wherein each chimeric polypeptide chain comprises a hinge region, an immunoglobulin G heavy chain constant region and an immunoglobulin tailpiece region, wherein the amino acid sequence of each polypeptide chain is altered as compared to the native sequences from which the hinge region, immunoglobulin G heavy chain constant region and the immunoglobulin tailpiece region are derived to possess an additional glycosylation site and to lack any cysteines from within the immunoglobulin G heavy chain constant region and immunoglobulin tailpiece region capable of forming extrinsic disulphide bonds. Suitably, the additional glycosylation site is in the hinge region corresponding to residue 1 of SEQ ID NO: 1 and there is no cysteine residue corresponding to residue 248 of SEQ ID NO: 1.

### Inhibition of Polymerisation of Proteins of the Invention

In particular proteins of the invention, the amino acid sequence and glycosylation of the tailpiece region is adapted, when compared to the sequence and glycosylation of the corresponding wild-type tailpiece (such as the IgM tailpiece), to inhibit polymerisation of the protein. Inhibition of polymerisation of such proteins may be demonstrated by either a decrease in the proportion of protein present in a polymeric form, or an increase in the proportion of the protein that is present in a monomeric form. This may be assessed with reference to the proportion of polymeric or monomeric protein found in an appropriate control protein. Such an appropriate control protein may comprise a wild-type tailpiece, for example the IgM tailpiece, and optionally, may comprise a native hinge region.

In a control protein (e.g. SEQ ID NO:1) lacking the adaptations of the proteins of the invention (for example, as exemplified by SEQ ID NO:2) monomers make up less than 20% of the total protein. In contrast, the inventors have found that more than 90% of a protein of the invention, such as that comprising the sequence disclosed in SEQ ID NO:2, is present in monomeric form under physiological conditions.

Thus in the case of the “cysteine residue molecules/proteins” of the invention, such as those comprising a sequence disclosed in any of SEQ ID NO:2 to SEQ ID NO: 8, in which the amino acid sequence, in particular key cysteine residues capable of forming extrinsic disulphide bridges, and glycosylation of the tailpiece region and hinge region are adapted as compared to the native/wild-type sequence, the adaptation may be demonstrated to be one that inhibits polymerisation if 90% or more of the protein is present in monomeric form under physiological conditions. Indeed, in a suitable embodiment, inhibition of polymerisation may result in 95% or more of a protein being present in monomeric form, for example, 96% or more, 97% or more, 98% or more, or even 99% or more. In a suitable embodiment, inhibition of polymerisation may result in substantially all of a protein of the invention being present in monomeric form under physiological conditions.

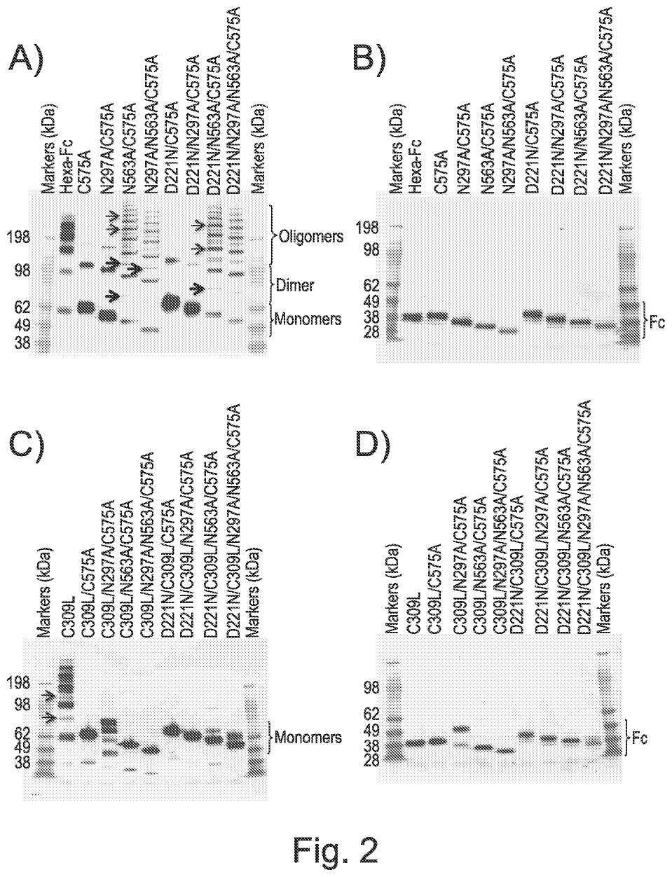

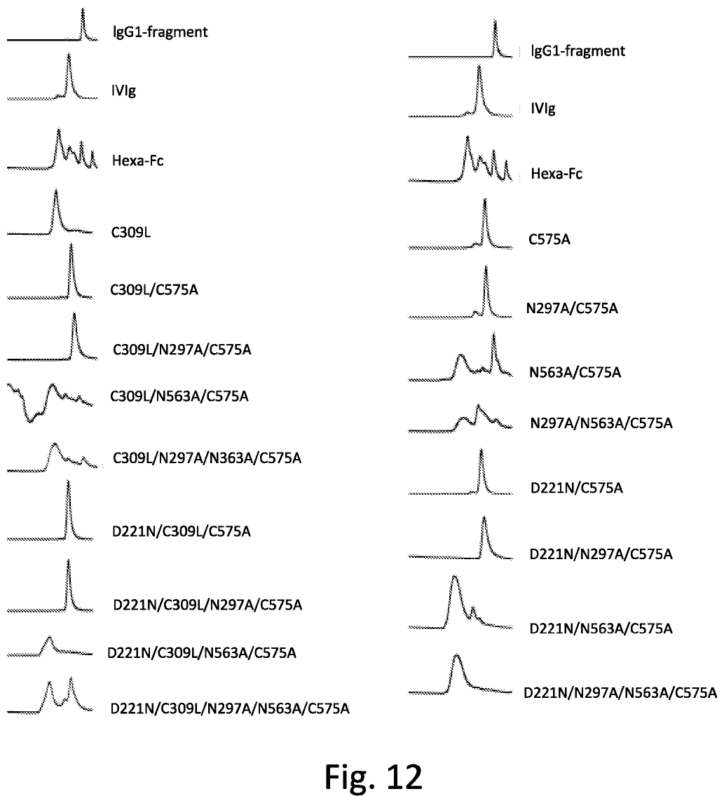

Suitable methods by which the proportion of polymeric or monomeric protein in a sample may be determined include size-exclusion chromatography (SEC) FIG. 12 and SDS-PAGE acrylamide gradient gels FIG. 2.

### IgG Sequences Suitable for Use in the Proteins of the Invention

The molecules/proteins of the invention, incorporate one, two or more immunoglobulin G heavy chain constant domains. In a suitable embodiment, each polypeptide chain incorporates two immunoglobulin G heavy chain constant domains. In a suitable embodiment, each polypeptide chain incorporates three immunoglobulin G heavy chain constant domains and an immunoglobulin G heavy chain variable region. In a suitable embodiment, the immunoglobulin G heavy chain constant region employed in the proteins of the invention are derived from an immunoglobulin selected from the group consisting of IgG1; IgG2; IgG3; and IgG4. In particular, the immunoglobulin G heavy chain constant region may be derived from IgG1. In a suitable embodiment, two immunoglobulin G heavy chain constant domains can be from the same immunoglobulin region (e.g. IgG1 or IgG4). In suitable embodiments, two immunoglobulin G heavy chain constant domains can be from different regions (e.g. domain 1 can be domain 1 from IgG1 and domain 2 could be domain 2 from IgG2, i.e. IgG1, CH2 and IgG2, CH3).

It will be appreciated that as long as they meet the requirement of forming an Fc receptor binding portion, the immunoglobulin G heavy chain constant regions utilised in proteins of the invention may include an alteration in their sequence as compared to the native sequences from which they are derived. Merely by way of example, a suitable protein of the invention may utilise IgG derived sequences that share at least 60%, at least 70%, at least 75%, at least 80%, at least 85%, at least 90%, at least 95%, at least 96%, at least 97%, at least 98%, or at least 99% sequence identity with the relevant native IgG sequence from which they are derived.

### Monomers of Proteins of the Invention

For the avoidance of doubt, in the context of the present disclosure, references to a “monomer” of a protein of the invention are intended to cover a molecule made up of two chimeric polypeptide chains associated with one another. Suitably, the two chimeric polypeptide chains may be linked by one or more inter-disulphide bonds formed between cysteine residues in the linker region. E.g. as discussed elsewhere in this application, there are a pair of cysteine residues in the hinge used in the exemplified proteins of the invention (DKTHTCPPCPAPELLGGP; SEQ ID NO: 28) that disulphide bond and help to keep the two polypeptide chains together to form the Fc monomer. Thus, it can be seen that, for present purposes, a “trimer” would be made up of three “monomers” as referred to above—a total of six chimeric polypeptide chains. A “hexamer” would consist of six monomers, and hence a total of twelve chimeric polypeptide chains.

### Compositions and Pharmaceutical Compositions of the Invention

The fifth aspect of the invention provides a composition comprising a protein of the invention (such as one according to the first, second or third aspects of the invention or another protein of the invention). In one embodiment, in such a composition, at least 95% of the protein of the invention incorporated in the composition is in monomeric form. In one embodiment, in such a composition, at least 95% of the protein of the first aspect of the invention incorporated in the composition is in monomeric form.

Suitably a composition of the fifth aspect of the invention may be a pharmaceutical composition, in which the protein is provided with a pharmaceutically acceptable carrier.

In a suitable embodiment of a composition comprising a protein of the first, second or third aspect of the invention, whether a pharmaceutical composition or otherwise, at least 96% or at least 97% of the protein of the first, second or third aspects of the invention incorporated in the composition is in monomeric form. Indeed, in a suitable embodiment, at least 98% or at least 99% of the protein of the first aspect, second or third aspects of the invention incorporated in the composition is in monomeric form. Suitably substantially all of the protein of the first, second or third aspects of the invention incorporated in such a composition may be in monomeric form.

In a suitable embodiment of a composition comprising a protein of the first aspect of the invention, whether a pharmaceutical composition or otherwise, at least 96% or at least 97% of the protein of the first aspect of the invention incorporated in the composition is in monomeric form. Indeed, in a suitable embodiment, at least 98% or at least 99% of the protein of the first aspect of the invention incorporated in the composition is in monomeric form. Suitably substantially all of the protein of the first aspect of the invention incorporated in such a composition may be in monomeric form.

In a suitable embodiment of a composition comprising another protein of the invention, whether a pharmaceutical composition or otherwise, the protein may be in multimeric form.

The pharmaceutical compositions according to the present invention, and for use in accordance with the present invention, may comprise, in addition to active ingredient, a pharmaceutically acceptable carrier. As used herein a pharmaceutically acceptable carriers includes: excipients, buffers, stabilisers or other materials well known to those skilled in the art. Such materials should be non-toxic and should not interfere with the efficacy of the active ingredient. The precise nature of the carrier will depend on the route of administration, which may be oral, or by injection, e.g. intravenous or ocular.

Examples of suitable pharmaceutical carriers are well known in the art and include phosphate buffered saline solutions, water, liposomes, various types of wetting agents, sterile solutions, etc. Compositions comprising such carriers can be formulated by well-known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Preparations for parenteral administration include sterile aqueous or non-aqueous solutions, and suspensions. Examples of non-aqueous solvents are propylene glycol, polyethylene glycol, and injectable organic esters such as ethyl oleate. Aqueous carriers include water, aqueous solutions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the composition might comprise proteinaceous carriers, like, e.g., serum albumin or immunoglobulin, in certain embodiments of human origin.

Pharmaceutical compositions for oral administration may be in tablet, capsule, powder, liquid or semi-solid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Solid formulations may be produced by lyophilisation, spray drying, or drying by supercritical fluid technology. Topical formulations may include viscosity modifying agents, which prolong the time that the drug is resident at the site of action. In certain embodiments, protein of the invention may be prepared with a carrier that will protect the protein against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Topical administration could be to the skin or other body part, such as the eye

A composition may be administered alone or in combination with other treatments, either simultaneously or sequentially dependent upon the condition to be treated

Many methods for the preparation of such formulations are known to those skilled in the art. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

### Medical Uses of the Proteins of the Invention

The proteins of the invention, for example the cysteine modified proteins, the glycosylation adapted proteins of the haemagglutinin binding proteins, provided in a composition of the invention, may be used as a medicament.

Proteins of the invention, e.g. the cysteine residue molecules, may for example, be used as medicaments in IVIG or SCIG. Such medical uses of the proteins and compositions are of particular utility in the prevention or treatment of autoimmune or inflammatory diseases. Medical use of the proteins of the invention in this manner may be effective, irrespective of whether or not they are conjugated to a therapeutic payload.

Proteins of the invention, e.g. the haemagglutinin binding proteins, may be used as medicaments for the prevention or treatment of diseases mediated through binding of sialic acid-dependent receptors.

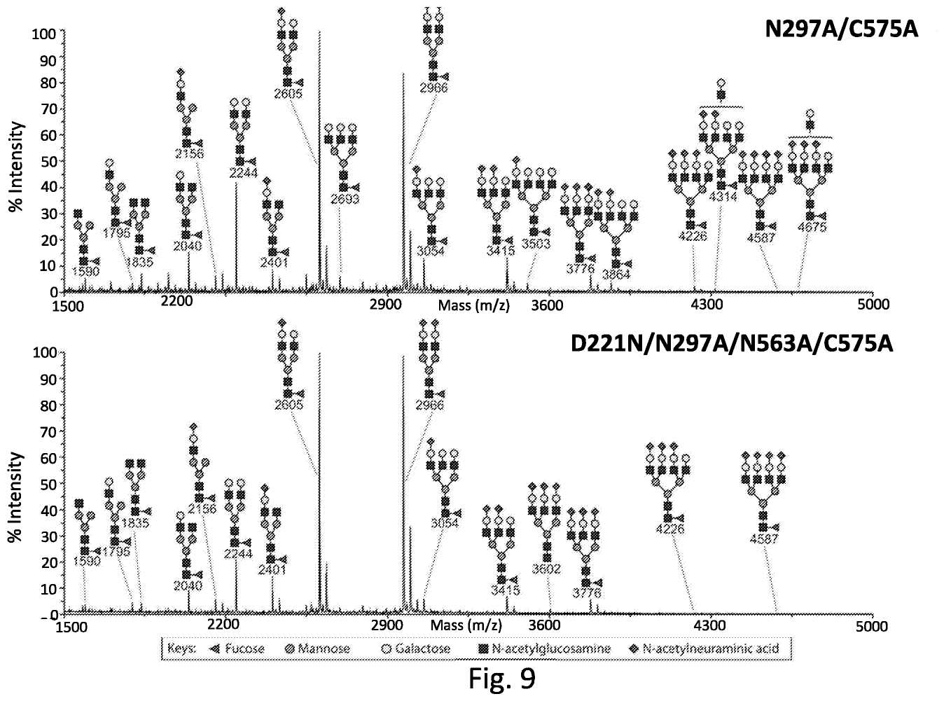

As mentioned elsewhere in this specification, proteins of the invention, e.g. cysteine residue molecules, and in particular proteins comprising the artificial glycosylation site at residue 1 of SEQ ID NO:1 (such as in the protein comprising SEQ ID NO:3), have the ability to bind sialic acid-dependent receptors, such as SIGLEC-1 (see Table 1), and thereby prevent other molecules from binding to the receptor. The inventors believe that the ability of the proteins to bind SIGLEC-1 and other sialic acid-dependent receptors is a result of their greater sialylation (FIG. 9).

In particular embodiments, the proteins of the invention are capable of binding at least one of the receptors selected from the group consisting of: SIGLEC-1, SIGLEC-2, SIGLEC-3, SIGLEC-4, CD23, dec-1, dec-2, DC-SIGN, CLEC-4A, CLEC-4D, MBL, MMR and DEC-205.

Proteins of the invention may be used as medicaments in diseases in which it is desirable to compete with, and thereby inhibit or prevent, the binding of other molecules to sialic acid dependent receptors, such as SIGLEC-1. Merely by way of example, binding of proteins of the invention to sialic acid dependent receptors such as SIGLEC-1 may have a therapeutic effect in inhibiting retrovirus binding to these receptors, thus preventing or treating retrovirus infections (such as HIV or T-cell leukaemia virus infections).

In particular embodiments, particular proteins of the invention are capable of binding haemagglutinin and thus may be useful as agents to inhibit pathogen infection, such as sialic-acid dependent viruses, e.g. influenza or Newcastle disease viruses. Particularly useful molecules include those that possess a glycan at or close to both termini of the molecule (such as at Asn221 and Asn 563), or within approximately 60 Å of each other. Particularly useful molecules are those that comprise a sequence as presented in SEQ ID NO: 7 and 59.

In particular embodiments, the proteins of the invention are capable of binding at least one of the Fc receptors selected from the group consisting of FcγRI, FcγRIIA, FcγRIIB, FcγRIIIA and FcγRIIIB.

By way of an example, D221N/C309L/N297A/N563A/C575A and D221N/C309L/N563A/C575A proteins of the invention bind inhibitory FcγRIIB more strongly than IgG1-Fc and as this receptor is known to be important in controlling inflammation (Lunemann, J. D. et al. Nat. Rev. Neurol. 11, 80-89 (2015); published online 6 Jan. 2015; doi:10.1038/nrneurol.2014.253) it may be useful therapeutically for treating inflammatory disease.

FcγRIIIA is known to be important in control of inflammation by IVIG through inhibitory ITAMs (Mkaddem et al, J Clin Immunol 2014 (34): S46-S50). Thus, molecules that bind FcγRIIIA (e.g. C309L/N563A/C575A) or that bind both FcγRIIIA and FcγRIIB (e.g. D221N/C309L/N563A/C575A) may also be useful in treating inflammatory disease.

FcγRIIIA can also bind immune-complexes and trigger activation e.g. ADCC by NK cells through non-inhibitory ITAMs, a well-known mechanism for the efficacy of anti-tumour mAbs (e.g. see Wang et al, Protein Cell. 9(1):63-73, 2018). Thus, molecules that bind FcγRIIIA may also trigger cell killing, whether that be tumours or pathogens.

Removal of C1q by IVIG has also been shown to be useful in treating neurological disease (Journal of Neuroimmunology 229 (2010) 256-262 and J Neurol Neurosurg Psychiatry 2011; 82:87e91. doi:10.1136/jnnp.2010.205856). Thus, molecules that bind C1q (e.g. D221N/C309L/N563A/C575A) should also be useful in controlling inflammation mediated by complement, especially as this mutant protein also binds inhibitory FcγRIIB and FcγRIIIA.

Mutants without effector function e.g. C309L/N297A/N563A/C575A may also be useful therapeutically. For example, they may be introduced into blocking mAbs wherein no extraneous activation of the immune response is required. Exemplar conditions in which one may wish to increase or decrease binding to FcγRs, glycan receptors or complement have been described in Wang et al., (Protein Cell 2018, 9(1):63-73, 2018, DOI 10.1007/s13238-017-0473-8).

Accordingly, such proteins may have a therapeutic effect in a number of diseases where binding to sialic acid dependent receptors and/or binding to Fcγ receptors and/or binding to C1q may be desirable.

Accordingly, the cysteine residue molecules/proteins of the invention, in particular those wherein an artificial glycosylation site has been introduced, may be used as medicaments in diseases in which preventing the binding of other molecules to sialic acid dependent receptors may have a therapeutic effect. Merely by way of example, preventing binding to SIGLEC-1 may have a therapeutic effect in retrovirus infections (such as HIV or T-cell leukaemia virus infections), or other conditions in which infectious agents bind via SIGLEC-1. Accordingly, the cysteine residue molecules/proteins of the invention, and in particular proteins comprising the artificial glycosylation site corresponding to that found at residue 1 of SEQ ID NO:2, may be used in the prevention or treatment of infections. Suitably proteins of the invention may be used in the prevention or treatment of retrovirus infections.

Proteins of the invention comprising or consisting of SEQ ID NO: 2-8, are particularly suited for the medical uses described above.

Other suitable examples of such diseases, which may benefit from prevention or treatment through medical use of the proteins of the invention, are considered below.

Those proteins of the invention that exhibit strong binding to FcγRIIIA and C1q (such as C309L/N563A/C575A) are expected to be particularly useful in the treatment of cancer; and so, may include a payload. The payload may be a binding-domain (e.g. antibody fragment) for a cancer antigen.

Further medical uses of the proteins of the invention may be selected with reference to a therapeutic payload conjugated to such proteins. A suitable therapeutic payload may be selected from the group consisting of an immune modulator, a drug, a polypeptide or protein, a carbohydrate, and a nucleic acid.

A suitable immune modulator may upregulate or downregulate components of the immune system.

A protein of the invention conjugated to an immune modulator which upregulates components of the immune system may be useful as a vaccine. By way of example an immune modulator which may be useful as a vaccine may be a pathogen-associated molecular pattern (PAMP) molecule or an antigen. Accordingly, the present invention provides the use of proteins of the invention as vaccines.

A protein of the invention conjugated to an immune modulator which down regulates the components of the immune system may be useful as a medicament for autoimmune diseases, for example rheumatoid arthritis.

An example of such an immune modulator which down regulates the components of the immune system is erythropoietin. Accordingly, it will be appreciated that in a suitable embodiment erythropoietin may be conjugated to a protein of the invention. Such a conjugated protein may be used in the prevention or treatment of an autoimmune disease.

The term “drug” as used herein refers to a compound with therapeutic activity, for example a small molecule, which may be conjugated to a protein of the invention. Merely by way of example, a suitable drug therapeutic payload may be one, such as monomethyl auristatin E, which may be useful in the treatment of cancer. Suitably, the drug, such as monomethyl auristatin E, may be further conjugated to an antibody. Accordingly, a protein of the invention may be conjugated to an anti-cancer drug, such as monomethyl auristatin E. Such a conjugated protein may be used in the prevention or treatment of cancer.

Merely by way of example, a suitable protein therapeutic payload for conjugation to a protein of the invention may be a cytokine receptor. Cytokine receptors may be useful for inhibiting disease-causing cytokines, by for example, binding such disease-causing cytokines, and thereby preventing them from pathogenically binding to cells.

A suitable carbohydrate payload to be conjugated to a protein of the invention may be, for example, hyaluronic acid.

A suitable nucleic acid payload to be conjugated to a protein of the invention may be, for example, unmethylated CpG oligodeoxynucleotide. Proteins of the invention conjugated in this manner are suitable for medical use as immunostimulants.

### Methods of Treatment Using the Proteins of the Invention

The proteins of the invention, for example provided in a composition of the invention, may be used as a medicament. The proteins may be conjugated to a therapeutic payload. Alternatively, they may be not conjugated to a therapeutic payload.

Such medical uses of the proteins and compositions thereof are of particular utility in the prevention or treatment of autoimmune or inflammatory diseases, whether or not the proteins are conjugated to a therapeutic payload.

Additionally, as already mentioned, the inventors have surprisingly found that the proteins of the invention, in particular proteins lacking cysteine residues in the IgG HC and tailpiece regions capable of forming extrinsic disulphide bridges and the artificial glycosylation site at residue 1 of SEQ ID NO:1 (such as the protein of SEQ ID NO:3), have the ability to bind sialic acid dependent receptors, for example, SIGLEC-1 receptors. The proteins may thereby prevent other molecules from binding to the receptor or may be used to trigger such receptors for therapeutic effect.

Therefore, proteins of the invention which are not conjugated to therapeutic payloads may be particularly useful in the prevention or treatment of diseases in which preventing the binding of other molecules to SIGLEC-1 may have a therapeutic effect. Merely by way of example, preventing binding to sialic acid-dependent receptors such as SIGLEC-1 may have a therapeutic effect in prevention or treatment of infections, such as retrovirus infections (such as Human Immunodeficiency Virus or T-Cell Leukaemia Virus infections) and/or other infections such as influenza and Newcastle disease viruses that are dependent on sialic-acid binding for entry into host cells.

Other suitable examples of such diseases, which may benefit from prevention or treatment through medical use of the proteins of the invention, are considered below.

The subject may be provided with a protein of the invention by any technique through which the subject will ultimately receive a therapeutically effective amount of the protein of the invention.

Thus, in a suitable embodiment the subject may be provided directly with the protein of the invention. In one embodiment the subject may, for example, be provided with a composition of the invention comprising the protein of the invention in monomeric form.

In another embodiment, the subject may be provided indirectly with monomeric protein. By way of example, in one embodiment, a nucleic acid according to the eighth aspect of the invention (a nucleic acid encoding a protein of the invention) may be administered to the subject, and the therapeutically effective amount of the protein of the invention provided by expression of the nucleic acid to yield the protein. Accordingly, in a tenth aspect, the present invention provides a nucleic acid in accordance with the eighth aspect of the invention for use as a medicament. The medical use of nucleic acids of the invention in this manner may be of benefit in the applications described with reference to the medical uses of proteins of the invention

Diseases that May be Prevented or Treated

Most glycan receptors including the siglecs are inhibitory receptors thus molecules that cross-link these receptors through multiple glycans attached to the Fc-fragment may be useful in controlling unwanted inflammation in therapies.

Suitable autoimmune or inflammatory diseases for prevention or treatment using the proteins of the invention include those that are treatable with IVIG. These may be diseases which are currently routinely treated with IVIG or in which IVIG has been found to be clinically useful, such as autoimmune cytopenias, Guillain-Barré syndrome, myasthenia gravis, anti-Factor VIII autoimmune disease, dermatomyositis, vasculitis, and uveitis. IVIG is typically used to treat idiopathic thrombocytopenic purpura (ITP), Kawasaki disease, Guillain-Barré syndrome and chronic inflammatory demyelinating polyneuropathy. IVIG may also be used to treat a diverse array of other autoimmune diseases which are non-responsive to mainstay therapies, including arthritis, diabetes, myositis, Crohn's colitis, and systemic lupus erythematosus.

Autoimmune or inflammatory diseases suitable for treatment include autoimmune cytopenia, idiopathic thrombocytopenic purpura, rheumatoid arthritis, systemic lupus erythematosus, asthma, Kawasaki disease, Guillain-Barré syndrome, Stevens-Johnson syndrome, neuromyelitis optica, Crohn's colitis, diabetes, chronic inflammatory demyelinating polyneuropathy myasthenia gravis, anti-Factor VIII autoimmune disease, dermatomyositis, vasculitis, uveitis, complement-mediated diseases, and Alzheimer's disease. It will be appreciated that autoimmune or inflammatory diseases such as those listed above may be treatable by a protein of the invention without a therapeutic payload. In such an embodiment treatment may be provided by IVIG or SCIG. Alternatively, or additionally, the diseases may be treatable by a protein of the invention conjugated to a therapeutic payload. Such a therapeutic payload may be, for example, an immune modulator which down regulates the components of the immune system.

Conditions to be treated may include an inflammatory disease with an imbalance in cytokine networks, an autoimmune disorder mediated by pathogenic autoantibodies, complement, or autoaggressive T cells, or an acute or chronic phase of a chronic relapsing autoimmune, inflammatory, or infectious disease or process. In addition, other medical conditions having an inflammatory component are included, such as Amyotrophic Lateral Sclerosis, Huntington's Disease, Alzheimer's Disease, Parkinson's Disease, Myocardial Infarction, Stroke, Hepatitis B, Hepatitis C, Human Immunodeficiency Virus associated inflammation, T-cell leukaemia virus associated inflammation, adrenoleukodystrophy, and epileptic disorders especially those believed to be associated with postviral encephalitis including Rasmussen Syndrome, West Syndrome, and Lennox-Gastaut Syndrome.

Conditions to be treated may be hematoimmunological diseases, e.g., Idiopathic Thrombocytopenic Purpura, alloimmune/autoimmune thrombocytopenia, Acquired immune thrombocytopenia, Autoimmune neutropenia, Autoimmune hemolytic anemia, Parvovirus B19-associated red cell aplasia, Acquired antifactor VIII autoimmunity, acquired von Willebrand disease, Multiple Myeloma and Monoclonal Gammopathy of Unknown Significance, Aplastic anemia, pure red cell aplasia, Diamond-Blackfan anemia, hemolytic disease of the newborn, Immune-mediated neutropenia, refractoriness to platelet transfusion, neonatal post-transfusion purpura, hemolytic uremic syndrome, systemic Vasculitis, Thrombotic thrombocytopenic purpura, or Evan's syndrome.

Alternatively, a neuroimmunological disease may be treated, e.g., neuritis, Guillain-Barré syndrome, Chronic Inflammatory Demyelinating Polyradiculoneuropathy, Paraproteinemic IgM demyelinating Polyneuropathy, Lambert-Eaton myasthenic syndrome, Myasthenia gravis, Multifocal Motor Neuropathy, Lower Motor Neuron Syndrome associated with anti-GM1 antibodies, Demyelination, Multiple Sclerosis and optic neuritis, Stiff Man Syndrome, Paraneoplastic cerebellar degeneration with anti-Yo antibodies, paraneoplastic encephalomyelitis, sensory neuropathy with anti-Hu antibodies, epilepsy, Encephalitis, Myelitis, Myelopathy especially associated with Human T-cell lymphotropic virus-1, Autoimmune Diabetic Neuropathy, or Acute Idiopathic Dysautonomic Neuropathy, and Alzheimer's disease.

A rheumatic disease may be treated, e.g., Kawasaki's disease, Rheumatoid arthritis, Felty's syndrome, ANCA-positive Vasculitis, Spontaneous Polymyositis, Dermatomyositis, Antiphospholipid syndromes, Recurrent spontaneous abortions, Systemic Lupus Erythematosus, Juvenile idiopathic arthritis, Raynaud's, CREST syndrome or Uveitis.

A dermatoimmunological disease may be treated, e.g., Epidermal Necrolysis, Gangrene, Granuloma, Autoimmune skin blistering diseases including Pemphigus vulgaris, Bullous Pemphigoid, and Pemphigus foliaceus, Vitiligo, Streptococcal toxic shock syndrome, Scleroderma, systemic sclerosis including diffuse and limited cutaneous systemic sclerosis, Atopic dermatitis or steroid dependent Atopic dermatitis.

A musculoskeletal immunological disease may be treated, e.g., Inclusion Body Myositis, Necrotizing fasciitis, Inflammatory Myopathies, Myositis, Anti-Decorin (BJ antigen) Myopathy, Paraneoplastic Necrotic Myopathy, X-linked Vacuolated Myopathy, Penacillamine-induced Polymyositis, Atherosclerosis, Coronary Artery Disease, or Cardiomyopathy.

A gastrointestinal immunological disease may be treated, e.g., pernicious anemia, autoimmune chronic active hepatitis, primary biliary cirrhosis, Celiac disease, dermatitis herpetiformis, cryptogenic cirrhosis, Reactive arthritis, Crohn's disease, Whipple's disease, ulcerative colitis, or sclerosing cholangitis.

The disease may be, for example, post-infectious disease inflammation, Asthma, Type 1 Diabetes mellitus with anti-beta cell antibodies, Sjogren's syndrome, Mixed Connective Tissue Disease, Addison's disease, Vogt-Koyanagi-Harada Syndrome, Membranoproliferative glomerulonephritis, Goodpasture's syndrome, Graves' disease, Hashimoto's thyroiditis, Wegener's granulomatosis, micropolyarterits, Churg-Strauss syndrome, Polyarteritis nodosa, or Multisystem organ failure.

An exemplary disease for treatment is idiopathic thrombocytopenic purpura (ITP).

It will be appreciated that conditions, such as those listed above, that are capable of treatment by IVIG may also be treated by SCIG. Accordingly, the use of the proteins or compositions of the invention in SCIG treatment of these conditions is also provided by the present invention.

The inventors believe that the proteins of the invention may lend themselves to use in improved activating and/or tolerogenic vaccines. Such vaccines may be suitable for use in individuals where activation of complement activation may not be desirable.

The proteins of the invention may be useful in the prevention or treatment of diseases which are mediated by the SIGLEC-1 receptor. Such diseases include those, such as viral infections, in which an infectious agent binds via SIGLEC-1. Suitable viral infections include retrovirus infections caused by, for example, Human Immunodeficiency Virus, T-cell Leukaemia Virus.

Diseases mediated by the SIGLEC-1 receptor may be prevented or treated by the proteins of the invention regardless of whether they are conjugated to a therapeutic payload or not. Proteins of the invention which are not conjugated to a therapeutic payload may prevent or treat the disease by blocking SIGLEC-1 receptors through competitive binding. Alternatively, proteins of the invention, whether conjugated to a therapeutic payload or not, may achieve therapeutic activity by triggering effector functions from such receptors. Proteins of the invention which are conjugated to a therapeutic payload, may prevent or treat the disease through the therapeutic effect of the therapeutic payload, or through a combination of the therapeutic effect of the therapeutic payload and blocking of SIGLEC-1 receptors through competitive binding.

As mentioned elsewhere in the specification, those proteins of the invention wherein inter-chain disulphide bond forming cysteines residues present in the native sequences of IgG heavy chain constant region and the tailpiece regions have been lost and an additional glycosylation site has been introduced (for example as found in SEQ ID NO:3) represent particularly suitable embodiments for use in the prevention or treatment of diseases modulated by siglecs, e.g. SIGLEC-1.

The proteins of the invention, particularly those that are capable of binding haemagglutinin may be particularly suitable for use in preventing or treating viral infections where they can be used either on their own or in combination with IVIG treatments, such as with a neutralising monoclonal antibody or IVIG-enriched for influenza reactivity.

### Nucleic Acids of the Invention

The eighth aspect of the invention provides nucleic acids that encode the proteins of the invention. In a suitable embodiment, the nucleic acids may encode a chimeric polypeptide, wherein the cysteine corresponding to that at position 575 of IgM (equivalent to C248 of SEQ ID NO:1) is lost. In such an embodiment, the cysteine residue at position 248 (corresponding to SEQ ID NO: 1) may be substituted by an alanine residue. In a further embodiment, in addition to the cysteine residue adaptation, the chimeric polypeptide also comprises the artificial glycosylation site at the position corresponding to residue 1 of SEQ ID NO: 1.

The nucleic acid of the invention may be a DNA molecule encoding a protein of the invention. Alternatively, the nucleic acid of the invention may be an RNA molecule, encoding a protein of the invention.

Suitably, a nucleic acid of the invention may comprise SEQ ID NO: 13, which encodes a polypeptide of SEQ ID NO: 2.

In a suitable embodiment the nucleic acid of the invention may share at least 70% identity with SEQ ID NO: 12, at least 75% identity with SEQ ID NO: 12, at least 80% identity with SEQ ID NO: 12, at least 85% identity with SEQ ID NO: 12, at least 90% identity with SEQ ID NO: 12, at least at least 95% identity with SEQ ID NO:12, at least 96% identity with SEQ ID NO: 12, at least 97% identity with SEQ ID NO: 12, at least 98% identity with SEQ ID NO: 12, or at least 99% identity with SEQ ID NO: 12.

It will be appreciated that the nucleic acids of the invention may be incorporated in larger nucleic acid sequences, which will comprise regions that do not encode the proteins of the invention. Merely by way of example, a nucleic acid of the invention may be incorporated in an expression plasmid, such as pFUSE-hlgG1-Fc-TP-LH309/310CL or pFUSE-hlgG1-Fc-TP-L310H.

### Production of Proteins of the Invention

The proteins of the invention are suitable both as clinical candidates, and for large-scale manufacture. The Fc fragments are a well understood class of compound and have even been used to treat idiopathic thrombocytopenia (ITP) in children. They are also straightforward to manufacture using existing antibody pipelines available for chinese hamster ovary (CHO) cells, the manufacturing workhorse of the pharmaceutical industry.

The ninth aspect of the invention provides a method of producing a protein of the invention. These methods comprise expressing a nucleic acid in accordance with the sixth aspect of the invention in a host cell.

In a suitable embodiment, the host cell may be a eukaryotic host cell. In particular, a suitable eukaryotic expression host may be selected from the group consisting of yeasts (for example Pichia pastoris and Saccharomyces cerevisiae) and mammalian cell systems.

Suitable mammalian cell systems may be selected from the group consisting of HEK-293 cells, CHO-K1 cells, mouse-derived NSO cells and BHK cells. Other suitable mammalian cell systems will be known to the skilled person. It will be appreciated that suitable host cells will comprise a means for attaching glycans to the expressed proteins.

In a particular embodiment, the proteins of the invention are expressed in CHO-K1 cells.

It will also be appreciated that the type of cells in which the proteins of the invention are expressed in may impact upon the type of linkages formed between the protein of the invention and a sialic acid. By way of example, proteins of the invention which are produced in CHO-K1 cells, may only form α2,3 linkages, and therefore may only bind SIGLEC-1 (as opposed to SIGLEC-2) receptors, OR haemagglutinin from influenzas with a preference for α2,3-linked sialic acids.

However, the expression of proteins of the invention in other types of cells, for example human cells (such as HeLa or HEK cells), may allow the formation of α2,3 linkages and α2,6 linkages. The presence of an α2,6 linkage may enable the proteins of the invention to bind SIGLEC-2 receptor (also known as CD22). The ability of the proteins of the invention to bind SIGLEC-2 receptor may be especially useful in the treatment of autoimmune diseases, for example by IVIG or SCIG treatment.

Accordingly, it will be recognised that the cells in which a protein of the invention are to be expressed may be selected with reference to desired glycosylation to be achieved, and the intended therapeutic use of the protein.

The inventors believe that the proteins of the invention that form monomers offer various practical advantages. For example, they simplify the process of manufacturing, since the proteins of the invention are produced as uniform monomers. Thus, the step of selecting proteins of only a particular size may be eliminated. The fact the certain proteins have the ability to not polymerise, may also extend the shelf-life of products comprising those proteins of the invention, without the concern that they will polymerise and lose their biological activities.

### Certain Aspects of the Invention.

### 1. Glycosylation Adapted Proteins

As noted above, WO2016/009232 (Liverpool School of Tropical Medicine) discloses multimeric forms of chimeric proteins that have alterations/adaptations in the number of glycosylation sites. To form multimeric proteins, the proteins disclosed therein should lack either the Asn77 or Asn236 residue, but not both (see page 5, paragraph 2 of WO2016/009232).

Surprisingly, the inventors have discovered that chimeric proteins comprising an Fc-receptor binding portion of an immunoglobulin that lack (via substitution) either or both the Asn77 and Asn236 residue and cysteine248, display excellent binding to FcγRI, FcgRIIIA and FcγRIIB and bind C1q and C5b-9. Such molecules are therefore immune potentiators, which makes them particularly suitable as antigen delivery vehicles for purposes such as vaccination. See above with respect of binding to FcγRIIIA, FcγRI and C1q. Mutant C309L/N563A/C575A may be suitable for vaccination purposes. Thus, an antigen from a particular virus (such as influenza, Zika or HIV) could be attached to such chimeric Fc mutant (as the therapeutic payload) and used for vaccination.

Thus, according to a third aspect of the invention there is provided a chimeric Fc receptor binding protein comprising two chimeric polypeptide chains; wherein each chimeric polypeptide chain comprises an immunoglobulin G heavy chain constant region and an immunoglobulin tailpiece, wherein the amino acid sequence of each polypeptide chain is altered as compared to the native sequences from which the immunoglobulin G heavy chain constant region and the immunoglobulin tailpiece region are derived to possess at least two fewer glycosylation sites. Such proteins are herein referred to as “glycosylation adapted proteins”. In a particular embodiment, such proteins also lack the cysteine in the tail region (e.g. at position 575).

In one embodiment of the glycosylation adapted protein, in each polypeptide chain as compared to the native immunoglobulin sequences from which the immunoglobulin G heavy chain constant region and the immunoglobulin tailpiece region are derived, there is a loss of the asparagine residue at the position corresponding to residue 77 of SEQ ID NO:1 and residue 236 of SEQ ID NO:1. In one embodiment, the asparagine residue at the position corresponding to residue 77 of SEQ ID NO:1 has been substituted by an alanine. In one embodiment, the asparagine residue at the position corresponding to residue 236 of SEQ ID NO:1 has been substituted by an alanine. In other embodiment, such proteins also lack the cysteine residue at the position corresponding to residue 248 of SEQ ID NO:1. Examples of proteins of the invention comprising such a modification comprise a polypeptide chain with the sequence disclosed in any of SEQ ID NOs: 9 and 10.

In one particular embodiment of the glycosylation adapted protein, said chimeric Fc receptor binding protein possesses a cysteine residue at the location corresponding to position 89 in SEQ ID NO: 1.

In one particular embodiment of the glycosylation adapted protein, said chimeric Fc receptor binding protein lacks the cysteine residue at the location corresponding to position 248 in SEQ ID NO: 1.

In one particular embodiment of the glycosylation adapted protein, said chimeric Fc receptor binding protein is capable of binding FcγRI and FcγRIIB. In another embodiment, said chimeric Fc receptor binding protein is capable of binding C1q. In another embodiment, said chimeric Fc receptor binding protein is capable of binding C1q and C5b-9.

Each chimeric polypeptide chain also comprises a hinge region. In a suitable embodiment, the hinge region may be located at the N-terminus of the Fc receptor binding portion. The hinge region may be a natural or synthetic hinge region as described further herein.

In another embodiment, each chimeric polypeptide chain also comprises a linker region between the immunoglobulin G heavy chain constant region and the immunoglobulin tailpiece region. Suitable linker regions are elsewhere in this specification.

In another embodiment, one or both chimeric polypeptide chains of the glycosylation adapted proteins of the invention comprise a therapeutic payload moiety. Suitable therapeutic payloads are described elsewhere in this specification. Suitably the payload may be conjugated to the Fc receptor binding portion of the protein of the invention. Alternatively, it may be conjugated to the hinge region of the protein of the invention.

An example of a glycosylation adapted protein comprises the amino acid sequence in SEQ ID NO: 10. The protein of SEQ ID NO: 10 is a particularly useful example of a protein that is highly suited to medical uses and methods in which it is desired for proteins to bind to FcγRIIB receptors (for example to deliver payloads to immune cells expressing these receptors, e.g. B cells).

Each of the aspects applicable to the cysteine residue molecules/proteins of the invention can be applied to the glycosylation adapted proteins described in this section.

Thus, according to another aspect there is provided a composition comprising a glycosylation adapted protein described herein.

According to another aspect of the invention there is provided a composition comprising a glycosylation adapted protein described herein for use as a medicament.

Such glycosylation adapted proteins or compositions may be used as medicaments in the prevention and/or treatment of infectious diseases. In particular, they can be used as vaccines to present antigens to the body. Suitable examples of such diseases are viruses, such as HIV, that use sialic acid receptors e.g. Siglec-1 to modulate infectivity and subsequent immune responses in vivo (Hammonds et al., PLoS Pathog. 2017 Jan. 27; 13(1):e1006181. doi: 10.1371/journal.ppat.1006181. eCollection 2017 January).

Alternatively, proteins or compositions of the invention may be used as medicaments in the prevention and/or treatment of diseases mediated through the binding of FcγRI and/or FcγRIIB. Diseases mediated through the binding of FcγRI and/or FcγRIIB are discussed elsewhere in this specification (see also: Akinrinmade O A, Chetty S, Daramola A K, Islam M U, Thepen T, Barth S. Biomedicines. 2017 Sep. 12; 5(3). pii: E56. doi: 10.3390/biomedicines5030056. Review).

The method of preventing or treating a disease involves providing a therapeutically effective amount of a glycosylation adapted protein or a composition containing it to a subject in need of such prevention or treatment. The subject may be a human subject. The administration can be IVIG or SCIG.

According to another aspect there is provided a nucleic acid encoding such a glycosylation adapted protein.

According to another aspect there is provided a method of producing a glycosylation adapted protein as described above, the method comprising expressing a nucleic acid encoding such a glycosylation adapted protein in a host cell.

### 2. Cys309 Adapted Proteins

Another type of protein of the invention are those that lack cysteine residues in the heavy chain constant region capable of forming extrinsic disulphide bridges but possess at least one cysteine residue capable of forming an extrinsic disulphide bridge in the tailpiece region. An example of a such a protein comprises the amino acid sequence shown in SEQ ID NO: 11.

Thus, according to another aspect of the invention there is provided a chimeric Fc receptor binding protein comprising two chimeric polypeptide chains; wherein each chimeric polypeptide chain comprises an immunoglobulin G heavy chain constant region, an immunoglobulin tailpiece region and a hinge region, wherein the amino acid sequence of each polypeptide chain lacks a cysteine residue in the heavy chain constant region capable of forming extrinsic disulphide bridges but possess at least one cysteine residue capable of forming an extrinsic disulphide bridge in the tailpiece region.

An example of a protein of the invention comprising such a modification comprises a polypeptide chain with the sequence disclosed in SEQ ID NOs: 4, 6 or 11; each of which display strong binding to FcγRI and FcγRIIIA.

In one embodiment, such a Cys309 adapted protein is capable of binding FcγRI and FcγRIIIA. The ability to bind FcγRI and FcγRIIIA is desirable in anti-cancer agents and vaccines.

Each of the aspects applicable to the cysteine residue molecules/proteins of the invention can also be applied to the Cys309 adapted proteins described in this section.

### 3. Haemagglutinin Binding Molecules

Another type of protein of the invention are those that are capable of binding haemagglutinin. Such molecules possess a sugar moiety at or close to each terminus of the protein. Molecules that possess Asn221 and Asn563 are particularly suitable.

Such molecules are predicted to be suitable for the treatment or prevention of diseases mediated by pathogens that access the cell via sialic acid receptors, such as influenza.

Current medicines for influenza have limited efficacy: Oseltarnivir (Tamiflu™) and zanamivir cause small reductions in the time to first alleviation of influenza symptoms in adults. The use of oseltamivir increases the risk of nausea, vomiting, psychiatric events in adults and vomiting in children. Oseltamivir has no protective effect on mortality among patients with 2009A/H1N1 influenza. Prophylaxis with either neuraminidase inhibitor (NI) may reduce symptomatic influenza in individuals and in households. The balance between benefits and harms should be considered when making decisions about use of NIs for either prophylaxis or treatment of influenza. All available anti-influenza drugs e.g. Oseltamivir are small molecule inhibitors of the viral neuraminidase. This is their sole mechanisms of action. Furthermore, these types of medicines are not without risks. For example, life-threatening abnormal behaviour has been documented in numerous studies, including a recent study in children 10-19 years of age. See PLoS One 2015 Jul. 1; 10(7):e0129712. doi: 10.1371/journal.pone.0129712. eCollection 2015. Because Oseltamivir is currently a black triangle drug, under intensive surveillance by the alternatives. There are also supply issues with Oseltamivir as it is derived from natural products meaning that it has not always been available for mass use.

Thus, according to a second aspect of the invention there is provided a chimeric Fc receptor binding protein comprising two chimeric polypeptide chains; wherein each chimeric polypeptide chain comprises an immunoglobulin G heavy chain constant region, an immunoglobulin tailpiece region and a hinge region, wherein the amino acid sequence of each polypeptide chain possess sugar moiety at or close to the N-terminus (such Asn-221) and a sugar moiety at or close to the C-terminus (such as Asn 563 or Asn573).

By at or close to we mean the terminal amino acid or one that is within 12, such as within 8 amino acids or within 5 amino acids of the terminus.

Such molecules may be useful as pathogen blockers, particularly to block sialic acid-dependent viruses such as influenza (Flu) and Newcastle disease virus.

Examples of other pathogens that can be blocked include: gram positive bacteria such as Streptococcus pneumoniae, Neisseria meningitidis, E. coli K1 and Campylobacter jejuni; or negative bacteria such as Pseudomonas aeruginosa, Haemophilus influenzae, Haemophilus ducrey, Pasteurella multocida and; parasites such as Trypanosoma cruzi and Leishmania donovani and viruses such as: influenza, Porcine Reproductive and Respiratory Syndrome Virus, rotavirus, Paramyxovirus (such as Newcastle disease virus and rubulavirus), norovirus, enterovirus, rotavirus, polyomavirus (such as e.g. Merkel cell polyomavirus), coronaviruses (including Mers and Sars), adenovirus (such as Ad37) and lentivirus (such as HIV-1).

In a particular embodiment the molecule comprises an Asn at the N-terminus and an Asn at the C-terminus −3 position. The C-terminus −3 position is the third amino acid from the C-terminus. The sugar moiety needs to be added here because the golgi apparatus require the NXS/T motif in order to attach a sugar to the Asn (N).

According to another aspect of the invention there is provided a chimeric Fc receptor binding protein comprising two chimeric polypeptide chains; wherein each chimeric polypeptide chain comprises an immunoglobulin G heavy chain constant region, an immunoglobulin tailpiece region and a hinge region, wherein the amino acid sequence of each polypeptide chain possess sugar moiety at or close to the N-terminus (such Asn-221) and a sugar moiety at or close to the C-terminus (such as Asn 563) for use in the treatment or prevention of a disease mediated by a pathogen that rely on sialic acid receptors interactions. By sialic acid interactions we mean the pathogen, e.g. virus or bacterium, binds to host sialic acid receptors.

According to another aspect of the invention there is provided a chimeric Fc receptor binding protein comprising two chimeric polypeptide chains; wherein each chimeric polypeptide chain comprises an immunoglobulin G heavy chain constant region, an immunoglobulin tailpiece region and a hinge region, wherein the amino acid sequence of each polypeptide chain possess sugar moiety at or close to the N-terminus (such Asn-221) and a sugar moiety at or close to the C-terminus (such as Asn 563) for use in the treatment or prevention of viral infection, such as from influenza virus or Newcastle disease virus.

According to another aspect of the invention there is provided a chimeric Fc receptor binding protein comprising two chimeric polypeptide chains; wherein each chimeric polypeptide chain comprises a hinge region, an immunoglobulin G heavy chain constant region and a tailpiece region, and wherein each polypeptide chain comprises two glycosylation sites located approximately 60 Å apart. In this aspect approximately means ±10Δ, such as ±5 Å. Such molecules can be also be referred to as double sugar molecules. In a particular embodiment, the tailpiece region is a polypeptide of between 12 and 26 amino acids in length that includes at least one glycosylation site. In one embodiment, the glycosylation site is at or near to the carboxy-terminus; such as at position −3.

According to another aspect of the invention there is provided a chimeric Fc receptor binding protein comprising two chimeric polypeptide chains; wherein each chimeric polypeptide chain comprises a hinge region, an immunoglobulin G heavy chain constant region and an immunoglobulin tailpiece region, and wherein each polypeptide chain comprises two glycosylation sites located approximately 60 Å apart. In this aspect approximately means ±10 Å, such as ±5 Å.

In a particular embodiment such double sugar molecules also lack any cysteine amino acids in the immunoglobulin G heavy chain constant region and in the tailpiece region (e.g. immunoglobulin tailpiece region) capable of forming inter-chain disulphide bonds. In a particular embodiment such double sugar molecules form monomers under physiological conditions.

The proteins of the invention that bind sialic acid receptors are predicted to be suitable in the prevention or treatment of diseases mediated by pathogens, such as viruses that need to bind sialic acid to get into a cell Unlike proteins that by nature of being coded for by DNA are mutatable, the carbohydrate binding contacts are unlikely to be mutated otherwise the pathogen (e.g. virus) cannot bind to the sialic acid required for entry. The molecules of the invention that bind these sialic acids on receptors make them unavailable for the pathogen (e.g. virus) to interact with via HA or neuraminidase. Without wishing to be bound by theory, by nature of containing sialic acid, the molecules of the invention may also compete for the viral neuraminidase and interfere with its action too. Thus, rather than have one mechanism of action, the molecules of the invention can potentially interfere with the virus by multiple mechanisms of action. Most small molecules drugs and antibodies that are mono-specific e.g. monoclonal antibodies or neuraminidase inhibitors cannot do this.

These molecules of the invention are also predicted to evade resistance mechanisms adopted by pathogens, e.g. viruses. For example, neutralising antibodies (whether raised by vaccines or used in passive therapy) that block the receptor binding site (RBS) on haemagglutinin (HA) or neuramidase (NA) also contact adjacent regions of these molecules. In other words, they have a large surface area footprint on either HA or NA. These footprint regions show high diversity in their sequences among different strains, which is why RBS reactive antibodies are usually strain-specific, making a constant reformulation of vaccines on a yearly basis inevitable. Because the molecules of the invention are much smaller than intact antibodies and only have glycan binding contacts, the possibility for adjacent interactions is much smaller thus lowering the chances of escape mutations.

### Proteins of the Invention

As used herein, a “protein of the invention” is a chimeric Fc receptor binding protein that comprises one or more adaptations of the amino acid sequence as compared to the sequence found in native/wild-type immunoglobulins from which they are derived, as described herein. One category of proteins of the invention are the “cysteine residue molecules”. Another category of proteins of the invention are the “haemagglutinin binding proteins”. Another category of proteins of the invention are the “glycosylation adapted proteins” and another category of proteins of the invention are the “Cys309” adapted proteins. Thus, whilst the first aspect of the invention relates to the “cysteine residue molecule” proteins of the invention, the second aspect of the invention relates to the “haemagglutinin binding” proteins of the invention and the third aspect of the invention relates to the “glycosylation adapted” proteins of the invention, the fourth, fifth, sixth, seventh, eighth and ninth aspects relate to any protein of the invention, i.e., the “cysteine residue molecules”, the “glycosylation adapted proteins”, the “Cys309 adapted proteins”, the “haemagglutinin binding” and any other protein of the invention.

The invention will now be further described with reference to the following Examples and accompanying figures, in which:

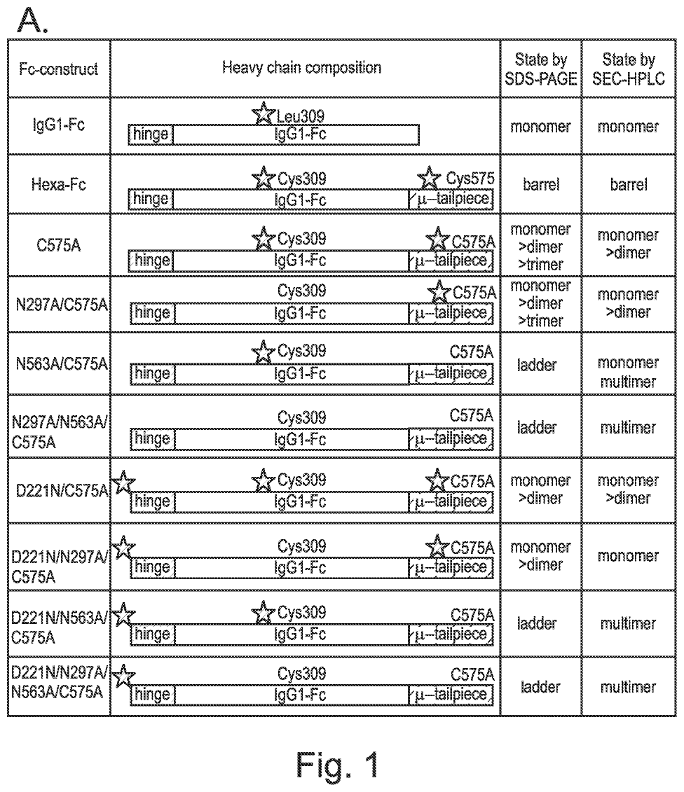

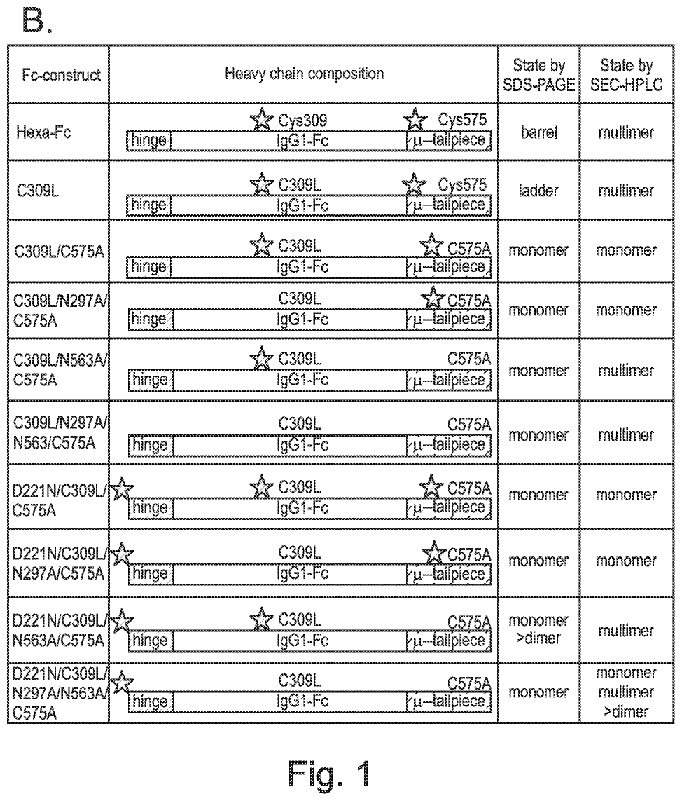

FIG. 1. Schematic showing the glycan and cysteine mutants generated.
A) Schematic showing the various hexa-Fc glycan mutants in which Cys575 is mutated to alanine to create the C575A panel of mutants. Red stars indicate the hinge Asn221, the Cg2 Asn297, and the tailpiece Asn563 glycan sites.
B) Schematic showing the C575A panel of glycan mutants from (A) in which Cys309 and Leucine310 is additionally changed to leucine and histidine as found in the native IgG1 Fc sequence to create the C309L/C575A panel of mutants. Red stars indicate the hinge Asn221, the Cg2 Asn297, and the tailpiece Asn563 glycan sites.

FIG. 2. Characterization of mutant Fc-proteins by SDS-PAGE.

A, N563A/C575A, N297A/N563A/C575A formed laddered multimers (red arrows) with folding intermediates (blue arrows) that are different to those formed by the hexa-Fc-control. The Asn-563 competent mutants C575A and N297A/C575A run mainly as monomers with dimers and trimers also seen. Therefore, removal of Asn-563 favors multimerization in the presence of Cys-309 but absence of Cys-575. The addition of a NX(T/S) glycan sequon to these mutants to generate N-terminally glycosylated hinges (the D221N series of mutants) did not affect multimerization but increased the molecular mass of all mutants. B, the same mutants as in A but run under reducing conditions. The decreasing molecular masses seen in the Fc represent sequential loss of N-linked glycans. Thus, the N297A/N563A/C575A mutant has the smallest molecular mass because it has no glycans attached to the Fc, and D221N/C575A has the largest mass because it has three glycans attached. The types of glycans attached at Asn-221, Asn-297 and Asn-563 have been described previously²³. C, Substitution of Cys-309 with leucine in mutants shown in A to create the double-cysteine knockouts which run as monomers. Differing molecular masses are seen with C309L/N297A/C575A monomers, which most likely represent differential glycosylation of Asn-563. D, the same mutants as in C but run under reducing conditions. All proteins were run under either non-reducing or reducing conditions at 1 μg protein per lane of a 4-8% acrylamide gradient gel, transferred to nitrocellulose, and blotted with anti-human IgG-Fc (Sigma).

FIG. 3. Binding of C309L and C575A double-cysteine IgG1-Fc monomeric glycosylation mutants to glycan receptors.

Mutants lacking either the N297 and/or N563 glycans are severely restricted in their capacity to bind glycan receptors by ELISA. The addition of an N-linked sugar at position 221 into the Asn-297 and/or Asn-563 deficient mutants reinstates binding to all receptors investigates with the exception of MBL, MMR and DC-SIGN. Insertion of Asn-221 into the Asn-297/Asn563 competent mutant C309L/C575A enhances interactions to all the glycan receptors investigated. Error bars represent standard deviations around the mean value, n=2 independent experiments.

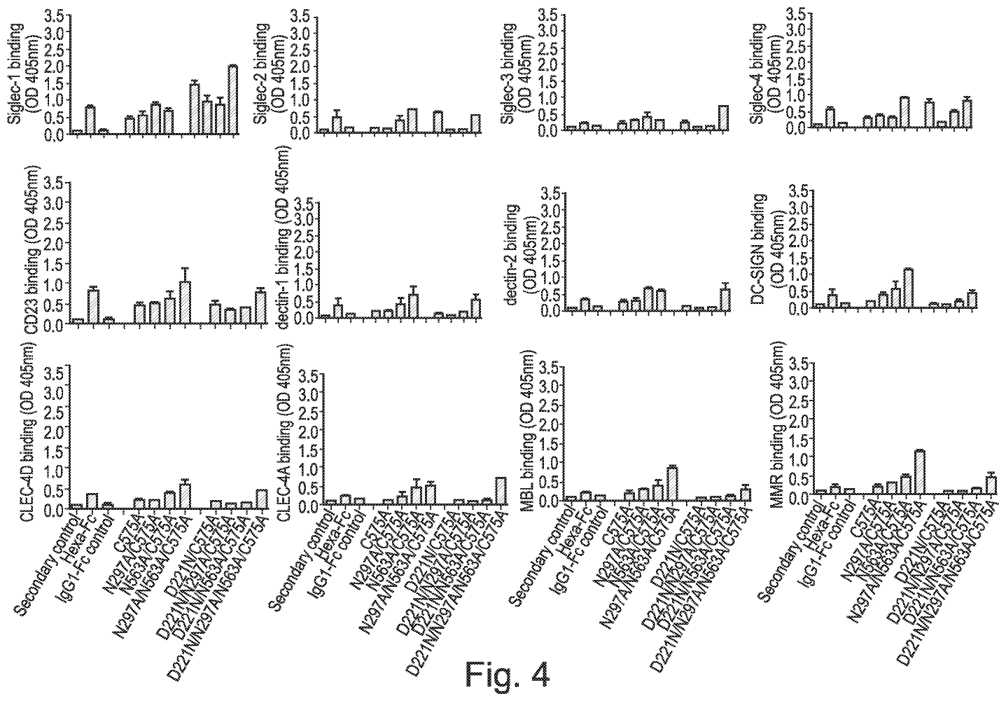

FIG. 4. Binding of C575A IgG1-Fc glycosylation mutants to glycan receptors.

Proteins with a predisposition to multimerize via Cys-309 interactions (as shown in FIG. 2A) are less able to engage glycan-receptors than equivalent mutants in which Cys-309 was also mutated to alanine (FIG. 3). With the exception of Siglec-1, the insertion of Asn-221 had no effect, or was detrimental to binding of glycan receptors by multimers. Error bars represent standard deviations around the mean value, n=2 independent experiments.

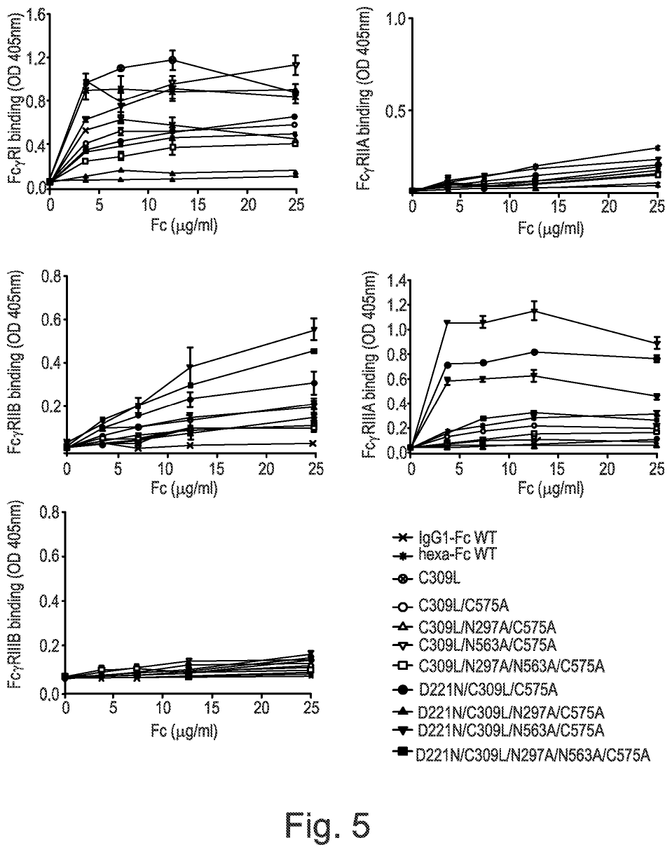

FIG. 5. Binding of C309L and C575A double-cysteine IgG1-Fc monomeric glycosylation mutants to classical FcγRs.

The D221N/C309L/N563A/C575A mutant shows enhanced binding to FcγRI, FcγRIIB and FcγRIIIA, while C309L/N563A/C575A only shows enhanced binding to FcγRI and FcγRIIIA attributable to the presence or absence of Asn-221. No improvement in binding was seen to either FcγRIIA or FcγRIIIB for any of the mutants studied. Error bars represent standard deviations around the mean value, n=2 independent experiments. Key: x-IgG1-Fc WT; *-hexa-Fc WT; crossed circle-C309L; open circle-C309L/C575A; open triangle-C309L/N297A/C575A; open inverted triangle (point down)—C309L/N563A/C575A; open square-C309L/N297A/N563A/C575A; black circle-D221N/C309L/C575A; black triangle-D221N/C309L/N297A/C575A; black inverted triangle-D221N/C309L/N563A/C575A; black square-D221N/C309L/N297A/N563A/C575A.

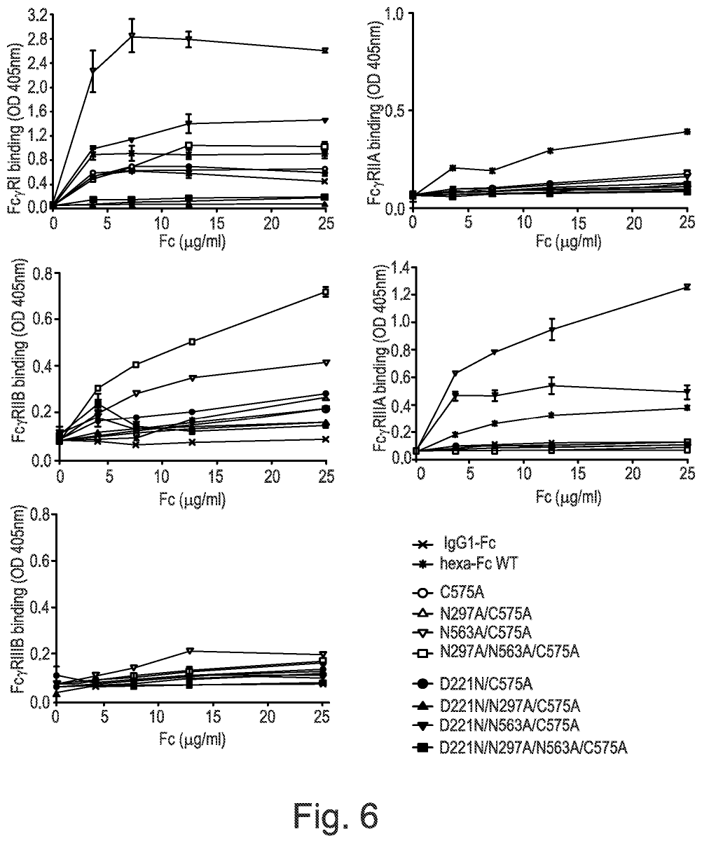

FIG. 6. Binding of C575A IgG1-Fc glycosylation mutants to classical FcγRs.

Mutant N563A/C575A with a predisposition to multimerize via Cys-309 interactions (as shown in FIG. 2A) binds strongly to FcγRI and FcγRIIIA and as seen with C309L/N563A/C575A monomers (FIG. 5) carrying the same mutations. Similarly, the D221N/N563A/C575A mutant shows enhanced binding to FcγRI and FcγRIII but in contrast to monomers containing the same mutations (D221N/C309L/N563A/C575A in FIG. 5) did not bind FcγRIIB. Thus, in multimers the presence of Asn-221 constrains interactions with FcγRIIB that are enhanced when Asn-221 is attached to monomers. As found with monomers no improvement in binding was observed to FcγRIIA or FcγRIIIB. Error bars represent standard deviations around the mean value, n=2 independent experiments. Key: x-IgG1-Fc WT; *-hexa-Fc WT; open circle-C575A; open triangle-N297A/C575A; open inverted triangle (point down)—N563A/C575A; open square-N297A/N563A/C575A; black circle-D221N/C575A; black triangle-D221N/N297A/C575A; black inverted triangle-D221N/N563A/C575A; black square-D221N/N297A/N563A/C575A.

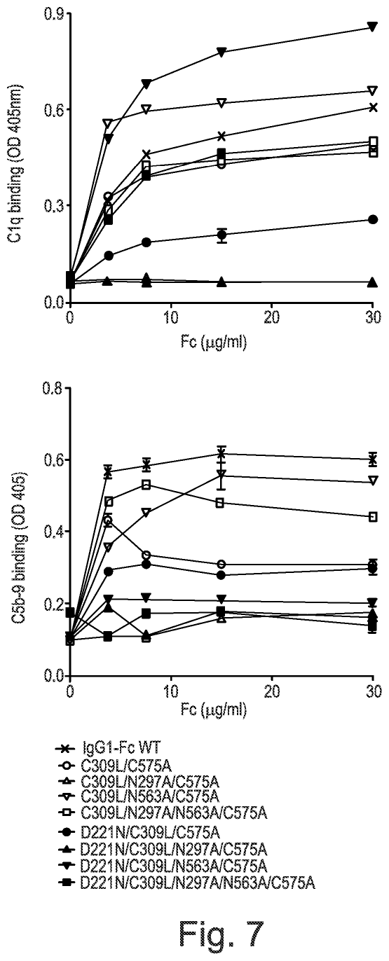

FIG. 7. Binding of C309L and C575A double-cysteine IgG1-Fc monomeric glycosylation mutants to complement.

Both the C309L/N563A/C575A and C309L/N297A/N563A/C575A mutants bound C1q and permitted C5b-9 deposition. Insertion of Asn-221 into both these mutants to create D221N/C309L/N563A/C575A and D221N/C309L/N297A/N563A/C575A allows C1q deposition but prevented subsequent C5b-9 deposition. This shows that the presence of the Asn-221 glycan, while allowing C1q to bind, blocks subsequent downstream activation of the classical pathway. Mutants in which only the Asn-297 glycan was removed, as in C309L/N297A/C575A or D221N/C309L/N297A/C575A were unable to bind C1q or fix C5b-9. Error bars represent standard deviations around the mean value, n=2 independent experiments. Key: x—IgG1-Fc WT; open circle-C309L/C575A; open triangle-C309L/N297A/C575A; open inverted triangle (point down)—C309L/N563A/C575A; open square-C309L/N297A/N563A/C575A; black circle-D221N/C309L/C575A; black triangle-D221N/C309L/N297A/C575A; black inverted triangle-D221N/C309L/N563A/C575A; black square-D221N/C309L/N297A/N563A/C575A.

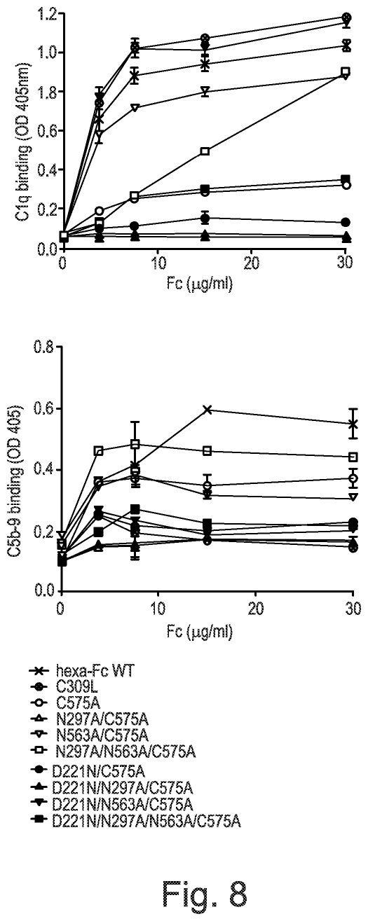

FIG. 8. Binding of C575A IgG1-Fc glycosylation mutants to complement.

With the exception of D221N/N563A/C575A the presence of Asn-221 inhibited binding to C1q, although all Asn-221 containing mutants including D221N/N563A/C575A were unable to fix C5b-9, and as previously observed for Cys-309/Cys-575 competent multimers²³. Error bars represent standard deviations around the mean value, n=2 independent experiments. Key: x—IgG1-Fc WT; crossed circle-C309L; open circle-C575A; open triangle-N297A/C575A; open inverted triangle (point down)—N563A/C575A; open square-N297A/N563A/C575A; black circle-D221N/C575A; black triangle-D221N/N297A/C575A; black inverted triangle-D221N/N563A/C575A; black square-D221N/N297A/N563A/C575A.

FIG. 9. MALDI-TOF MS profiles of permethylated N-glycans from N297A/C575A and D221N/N297A/N563A/C575A IgG1-Fc mutants.

The data were acquired in the positive ion mode to observe [M+Na]+ molecular ions. All the structures are based on composition and knowledge of biosynthetic pathways. Structures shown outside a bracket have not had their antenna location unequivocally defined.

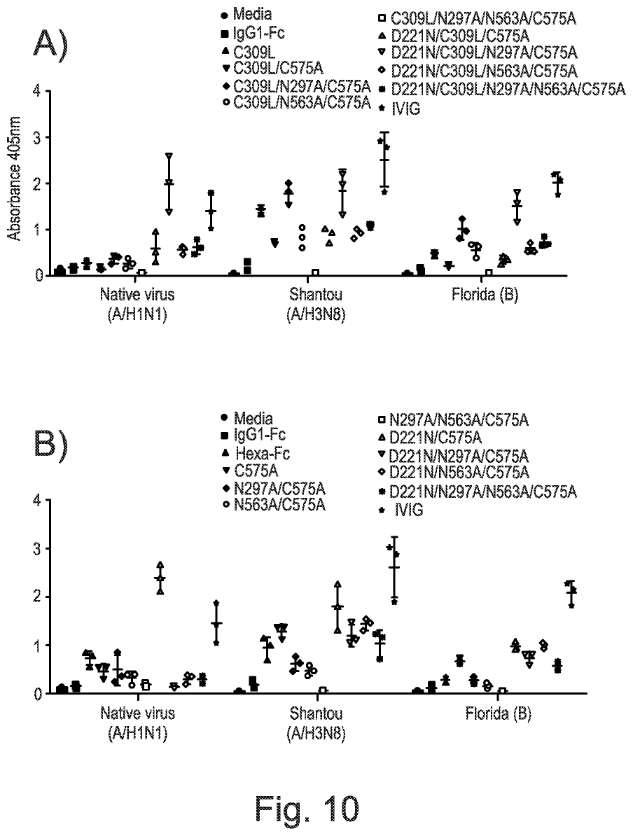

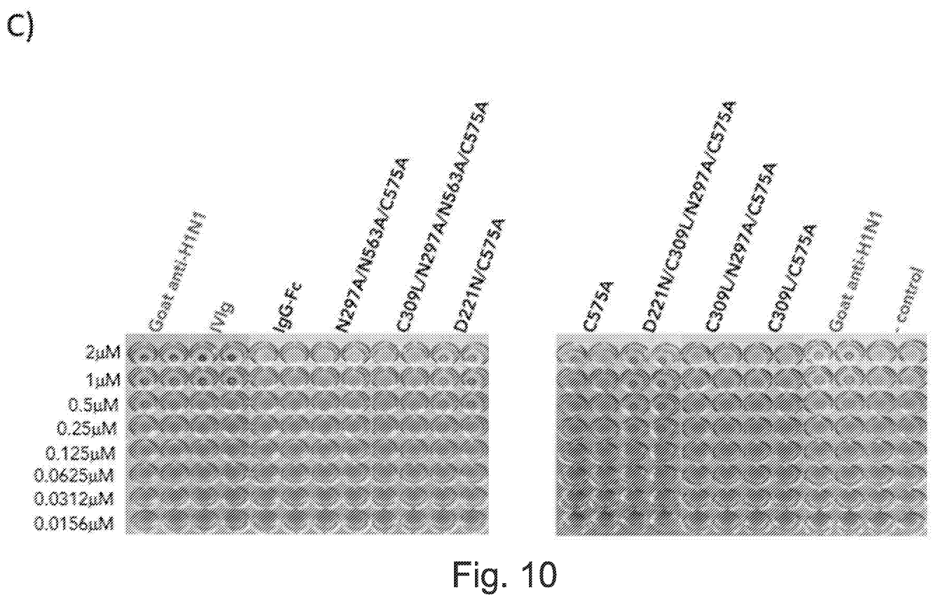

FIG. 10. Impact of Fc glycosylation.

(A) ELISA binding of the C575A/C309A panel and (B) the C575A panel of Fc glycosylation mutants to hemagglutinin. (C) impact of Fc glycosylation on hemagglutination inhibition. A constant amount of influenza A New Caledonia/20/99virus H1N1 was incubated with titrated amounts of the Fc glycan mutants and added to human O+ erythrocytes that were then allowed to sediment at room temperature for 1h. Non-agglutinated RBCs form a small halo. n=2 independent experiments.

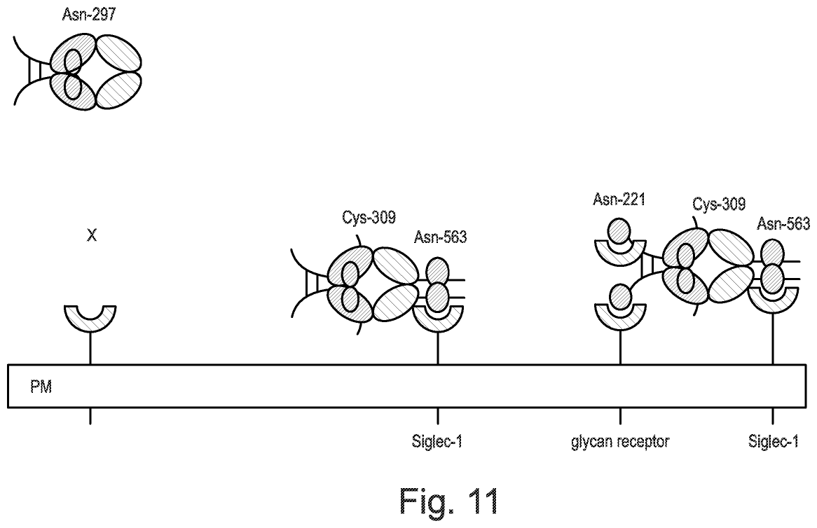

FIG. 11.

Model showing proposed cis interactions of variably glycosylated IgG1 Fc with glycan receptors. The glycan at Asn297 in IgG1 (left) is buried and unable to interact directly with receptors. However, monomers with glycans located at both the N- and C-terminal ends of the Fc (Asn221 and Asn563) are exposed (middle and right) and would therefore allow greater binding to receptors in cis that are favoured by sialic-acid-dependent receptors⁴¹.

FIG. 12.

SEC-HPLC chromatograms of C309L/C575A double cysteine knock-out mutants (left column) and C575A cysteine mutants (right column).

### EXAMPLES

### Introduction

Multimerised Fc and Fc-fusion proteins are increasingly being explored for novel drug and vaccine approaches. One potentially fertile area is their development as biomimetic replacements for intravenous immunoglobulin (IVIG) therapy. IVIG is a hugely successful biological with FDA approval for treating idiopathic thrombocytopenic purpura (ITP), Kawasaki disease, Guillain-Barré syndrome, Graves ophthalmopathy and numerous polyneuropathies. IVIG is increasingly viewed by clinicians as a last resort cure-all for a plethora of other diseases including: anemias, arthritides, lupus, transplant rejection, abortion, and chronic pain; especially when these are non-responsive to conventional therapies.

The global shortage and demand for IVIG is compounded by a number of other inadequacies with the current drug, the most significant being its dependency on human donors for its production, raising safety issues and greatly adding to cost. To add insult to injury <5% of the injected product (correctly glycosylated and/or oligomeric-Fc) is therapeutically active leading to a requirement for high dosages (2 g/kg). Consequently, IVIG is expensive and adverse events due to excessive IVIG loading not uncommon. There is therefore an urgent clinical need to develop synthetic replacements for IVIG for use in the clinic.

The mechanism of action of IVIG is incompletely understood. Although both Fab′₂ and Fc-mediated mechanisms may be involved, in humans the infusion of Fc fragments is sufficient to ameliorate ITP. IVIG suppresses harmful inflammation by engaging low-affinity inhibitory receptors and/or by forming complexes in vivo that allow IVIG to interact with these receptors with greater avidity, thus mediating more potent anti-inflammatory effects. The exact receptors or combinations of receptors involved are not definitively known, although both classical (type 1, e.g. FcγRIIB, FcγRIIIA) and non-classical (type 2, e.g FcγRs such as DC-SIGN, CD22, FcRL5) have been implicated in its therapeutic efficacy.

Based on the finding that Fc complexes can induce tolerance, a number of different approaches to Fc multimerization are being actively investigated. One approach utilizing the hinge region of human IgG2 generates laddered sequential multimers of diverse molecular weights when introduced into mouse IgG2a-Fc. The higher-order multimers, termed ‘Stradomers™’ bound strongly to low-affinity FcγRs and SIGN-R1, and were shown to protect animals from collagen-induced arthritis, ITP, inflammatory neuropathy, and autoimmune myasthenia gravis.

The inventors took an alternative approach to multimerization by fusing the 18 amino-acid tailpiece from oligomeric IgM, together with a Leu to Cys substitution at position 309, into the IgG1-Fc to create molecules termed Hexa-Fc. These molecules formed defined pentameric and hexameric structures whose binding to receptors was shown to be critically dependent on N-linked glycosylation. Glycosylation is important in increasing the solubility and in influencing interactions with both glycan- and Fc-receptors. Hexa-Fc contains two N-linked glycosylation sites at positions Asn297 in the Cγ2 domain and Asn563 in the eighteen amino acid IgM tailpiece of hexa-Fc. The hexameric Fc also binds the human neonatal receptor (FcRn), an interaction that is known to be critical to the maintenance of a long in vivo half-life and to enhanced immunogenicity. The efficacy of similar molecules in a mouse model of ITP has been reported in three patent applications (WO2015132364, WO2015132365 and WO2017129737).

Glycosylation is important for correct protein folding in the endoplasmic reticulum and for exporting correctly folded proteins to the Golgi for post-translational modifications. Attached glycans also increase the solubility of proteins and have been shown to influence significantly the interactions of IgG with both glycan- and Fc-receptors. Glycosylation of the only available carbohydrate attachment site (Asn297) in the Fc is essential for interactions with both type 1 and 2 receptors. The Fc glycans at Asn297 are typically biantennary complex types, exhibiting high levels of fucosylation of the core GlcNAc residue, partial galactosylation, and bisecting GlcNac. Of these structures, less than 20% are sialylated. The reason for the low levels of branching and terminal structures, such as sialic acid, are believed to result from constraints on Asn297 glycan processing imposed by the Fc protein backbone.

The anti-inflammatory properties of the Fc are lost after deglycosylation of IVIG, and a population of IgG-bearing α2,6-sialylated Fcs has been identified as making a significant contribution to the control of inflammation. Higher levels of sialylation also lead to longer serum retention times^{8,9.}

Indeed, the efficacy of sialylated Fc has generated an incentive to modify the existing glycans on Asn297, either by chemical means or through mutagenesis programs in the Fc protein backbone that disrupt the protein-Asn297 carbohydrate interface.

The human IgG1-Fc typically does not bind glycan receptors because the glycan attached to Asn-297 is largely buried within the cavity formed by the CH2-CH3 homodimer^{26,27}. The location and content of glycans attached at Asn-297 also modulates the affinity of the Fc for binding to the classical FcγRs, through conformational changes imparted to the FcγR binding region located in the lower hinge²⁸. Herein, the inventors show that these limitations to Asn-297-directed receptor binding can be overcome through a program of mutagenesis aimed at disrupting disulphide bonding while enhancing N-linked glycosylation within the IgG1-Fc (FIG. 1).

To this end, the inventors created a panel of human monomeric IgG1-Fc mutants (FIG. 1), through the deletion of critical disulphide bonds, and/or the insertion/deletion of N-linked asparagine attachment sites located within the previously described IgG-Fc multimer^{2,23,29,30} This approach not only yielded molecules with enhanced binding to low-affinity FcγRs, but also imparted binding to receptors not previously known to bind the IgG1-Fc, including Siglec-1, Siglec-2, Siglec-3, Siglec-4, CD23, Dectin-1, Dectin-2, CLEC-4A (DCIR), CLEC-4D, MMR, MBL and DEC-205. Furthermore, the inventors were able to identify monomeric mutants with limited ability to bind FcγRs (e.g. CL309-310LH/N297A/N563A/C575A), but with enhanced avidity for C1q, that may make attractive therapeutics for complement-mediated diseases.

Herein, the inventors show that the N-glycan at Asn297, whose sugars are enriched for high mannose and galactose when compared with IVIG, is essential for receptor binding by hexa-Fc. However, the glycans attached to the tailpiece Asn563 were found to be larger and more complex than those attached at Asn297 and were not critical to receptor binding as oligomers, but were essential in determining the type of oligomer formed. Removal of both Asn297 and Asn563 led to a significant drop in protein expression, inability to oligomerise, and a complete loss of receptor binding. These findings show the importance of N-linked glycosylation and the tailpiece in maintaining the structure and function of hexa-Fc, and as such, the translational potential of these molecules for either drug or vaccine applications.

Additionally, the inventors have taken an unexplored approach to modifying glycosylation by introducing, in various combinations, up to three additional N-linked glycosylation sites into exposed areas of the IgG1-Fc (FIGS. 1 and 9). Hexa-Fc typically contains two N-linked glycosylation sites at positions Asn297 in the Cγ2 domain and at Asn563 in the eighteen amino acid IgM tailpiece of hexa-Fc.

### Materials and Methods

Many of these methods and tools are also disclosed in Blundell et al. (Immunology. 202(5):1595-1611, 2019).

### Production of Glycosylation Mutants.

The generation of glycan mutants in all combinations has been described previously for the hexa-Fc that contains cysteines at both positions 309 and 575²³. To make the new mutants described in FIG. 1A in which Cys⁵⁷ was mutated to alanine, PCR overlap extension mutagenesis was used with a pair of internal mismatched primers 5′-ACCCTGCTTGCTCAACTCT-3′ (SEQ ID NO: 29)/3′-GGCCAGCTAGCTCAGTAGGCGGTGCCAGC-5′ (SEQ ID NO: 30) for each plasmid vector coding for a designated glycan modification. The parental plasmids used for these new PCR reactions have been described previously²³. The resulting C575A mutants were then further modified to remove Cys³⁰⁹/Leu³¹⁰ using primer pair 5′-TCACCGTCTTGCACCAGGACT-3′ (SEQ ID NO: 31)/3′-AGTCCTGGTGCAAGACGGTGA-5′ (SEQ ID NO: 32) to create the panel of double cysteine knockouts described in FIG. 1B. To verify incorporation of the desired mutation and to check for PCR-induced errors, the open reading frames of the new mutants were sequenced on both strands using previously described flanking primers²³

CHO-K1 cells (European Collection of Cell Cultures) were transfected with plasmid using FuGene (Promega), and positive clones were selected, expanded, and purified as previously described^{2,30} The following substitution mutations were constructed by PCR overlap extension mutagenesis from the wild-type vector (pFUSE-hIgG1-Fc-TP-LH309/310CL) as the template, using pairs of internal mismatched primers for each mutation as follows.

N297A:
(SEQ ID NO: 33)/
5′-GAGCAGTACGCCAGCACGTAC-3′
(SEQ ID NO: 34)
3′-CTCGTCATGCGGTCGTGCATG-5′;
N563A:
(SEQ ID NO: 35)/
5′-CCCTGTACGCCGTGTCCCTG-3′
(SEQ ID NO: 36)
3′-GGGACATGCGGCACAGGGAC-5′;
D221N:
(SEQ ID NO: 37)/
5′-GTTAGATCTAACAAAACTCAC-3′
(SEQ ID NO: 38)
3′-CAATCTAGATTGTTTTGAGTG-5′
C575A:
(SEQ ID NO: 39)/
5′-ACCCTGCTTGCTCAACTCT-3′
(SEQ ID NO: 40)
3′-GGCCAGCTAGCTCAGTAGGCGGTGCCAGC-5′
C309L:
(SEQ ID NO: 31)/
5′-TCACCGTCTTGCACCAGGACT-3′
(SEQ ID NO: 32)
3′-AGTCCTGGTGCAAGACGGTGA-5′

N297A/N563A: primer pair N563A was used on the N297A mutant plasmid;

D221N/N297A: primer pair N297A was used on D221N mutant plasmid;

D221N/N563A: primer pair N563A was used on the D221N mutant plasmid;

D221N/N297A/N563A: primer pair N563A was used on the D221N/N297A mutant plasmid;

N563A/C575A: primer pair C575A was used on the N563A mutant plasmid.

The following flanking primers were used in the overlap PCR. These are 5′-ACCCTGCTTGCTCAACTCT-3′ (SEQ ID NO: 43) and 3′-TGGTTTGTCCAAACTCATCAA-5′ (SEQ ID NO: 44) which are 71 or 22 base pairs upstream or downstream of the EcoRI/BglII and NheI (all from New England Biolabs) sites used in sub-cloning into the wild-type vector. DNA coding for the human IgA tailpiece (PTHVNVSVVMAEVDGTCY; SEQ ID NO: 45) was synthesised by EUROFINS and cloned as an AvrII/NheI fragment into pFUSE-hIgG1-Fc-TP-LH309/310CL. To verify incorporation of the desired mutation and to check for PCR-induced errors, the entire coding sequence of the new expression plasmids were sequenced on both strands using the same set of flanking primers. CHO-K1 cells (European Collection of Cell Cultures) were transfected with plasmid using FuGene (Promega) and positive clones selected, expanded and purified as previously described for hexa-Fc^{2,3}

### Receptor and Complement Binding Assays

Methods describing the binding of mutants to tetrameric human DC-SIGN (Elicityl), Siglec-1, Siglec-4, and Siglec-3 (Stratech Scientific) have all been described previously 2,30. The ELISA protocol used to detect lectin binding was also used for Siglec-2, CD23, dec-1, dec-2, clec-4a, clec-4d, MBL and MMR (Stratech Scientific or Bio-Techne), and has also been previously described²,30 Binding of C1q and C5b-9 have also been described previously^{2,30}.

ELISAs were used to investigate binding of Fc mutants to human FcγRI, FcγRIIA, FcγRIIB, FcγRIIIA, and FcγRIIIB (Bio-Techne). Receptors were coated down on ELISA plates (Nunc) in carbonate buffer pH 9 (Sigma-Aldrich) at 2 g/ml overnight at 4° C., unless alternatively specified. The plates were blocked in PBS/0.1% Tween-20 (PBST) containing 5% dried skimmed milk. The plates were washed three times in PBST before adding Fc mutant proteins at the indicated concentrations and kept at 4° C. overnight. The plates were washed as above and incubated for 2h with 1:500 dilution of an alkaline phosphatase-conjugated goat Fab′₂ anti-human IgG (Jackson Laboratories). The plates were washed and developed for 15 min with 100 l/well of a Sigmafastp-nitrophenyl phosphate solution (Sigma-Aldrich). The plates were read at 405 nm, and the data were plotted with GraphPad Prism.

### Binding to Hemagglutinin

ELISA plates were coated with 5 μg/mL recombinant HA from different influenza A and B viruses (BEI Resources) or native influenza A New Caledonia H1N1 virus (2B Scientific) in carbonate buffer pH 9 and left at 4° C. overnight. Plates were washed five times with TSM buffer (20 mM Tris-HCl, 150 mM NaCl, 2 mM CaCl2), 2 mM MgCl2), prior to blocking for 2h in 150 μl per well of TMS buffer containing 5% bovine serum albumin. After washing as before, 100 μl of Fc fragments at 30 μg/mL in TSM buffer was added in triplicate wells. Fc fragments were allowed to bind overnight at 4° C. Plates were washed five times with excess TSM buffer, prior to the addition of 100 μl per well of alkaline-phosphatase conjugated F(ab′)2 goat anti-human IgG1 Fcγ fragment-specific detection antibody diluted 1 in 500 in TMS buffer. Glycosylated Fc fragments that bound to the glycan receptors were left to bind the conjugated antibody for 1h at room temperature on a rocking platform. Plates were washed as above and developed for 10 minutes with 100 μl per well of p-Nitrophenyl phosphate. Plates were read at 405 nm using a LT-4500 microplate absorbance reader (Labtech), and the data plotted with GraphPad Prism.

### Hemagglutination Inhibition Assay

To determine the optimal virus-to-erythrocyte ratio, two-fold virus stock (2B Scientific) dilutions were prepared in U-shaped 96-well plates (Thermo Scientific). The same volume of a 1% human O+ red blood cell suspension (Innovative Research) was added to each well and incubated at RT for 60 min until erythrocyte pellets had formed in the negative control. After quantifying the optimal virus-to-erythrocyte concentration (4HA units), serial two-fold dilutions of Fc, control IVIG (GammaGard, Baxter Healthcare) or polyclonal goat anti-influenza H1N1(Bio-rad) were prepared, starting at a concentration of 2 μM, and mixed with 50 μl of the optimal virus dilution. After 30 min incubation at 4° C., 50 μl of the human erythrocyte suspension was added to all wells and plates incubated at RT for 1h, after which erythrocyte pellets could be observed in the positive controls.

### N-Glycomic Analysis

N-glycomic analysis was based on previous developed protocol with some modifications⁸⁴. Briefly, the N-glycans from 50 ug of each sample were released by incubation with NEB Rapid™ PNGase F and isolated from peptides using Sep-Pak C18 cartridges (Waters). The released N-glycans were permethylated, prior to Matrix-assisted laser desorption ionization (MALDI) MS analysis. Data were acquired using a 4800 MALDI-TOF/TOF mass spectrometer (Applied Biosystems) in the positive ion mode. The data were analyzed using Data Explorer (Applied Biosystems) and Glycoworkbench⁸⁵. The proposed assignments for the selected peaks were based on composition together with knowledge of the biosynthetic pathways.

### Results

### Disulphide Bonding and Glycosylation Influence the Multimerization States of Hexa-Fc

To determine the contribution of two N-linked glycosylation sites (Asn-297 and Asn-563) and two cysteine residues (Cys-309 and Cys-575) in the multimerization of hexa-Fc, we created two panels of glycosylation- and cysteine-deficient mutants by site-directed mutagenesis, using the previously described hexa-Fc as the template (FIG. 1)²³. We also inserted an N-linked attachment site at the N-terminus (D221N) to investigate the impact of additional glycosylation on Fc function (FIG. 1). Following transfection of these mutated IgG1-Fc DNAs into CHO-K1 cells, stable clonal cell lines were established, and the secreted Fcs were purified by protein A/G affinity chromatography. The purified IgG1-Fc proteins were analysed by SDS-PAGE and immunoblotting with anti-human IgG-Fc (FIG. 2A,B).

When analysed under non-reducing conditions (FIG. 2A), the C575A mutant migrated mostly as monomers (˜55 kDa), with a small proportion of dimer (˜110 kDa) and trimer (˜165 kDa). Insertion of a glycan at Asn-221 into the C575A mutant (to create D221N/C575A) resulted in loss of the trimer fraction and a decrease in the proportion of dimers observed, although the molecular weights of each of the species increased as a consequence of the additional N-terminally attached Asn-221 sugar (FIG. 2A,B).

Because we had previously shown that removal of the tailpiece glycan (Asn-563) in hexa-Fc led to the formation of dodecamers²³, we reasoned that a similar mutation introduced into the C575A backbone would also lead to enhanced dodecamer formation. Surprisingly, removal of Asn-563, as in the N563A/C575A, N297A/N563A/C575A, D221N/N563A/C575A and D221N/N297A/N563A/C575A mutants, led to the formation of a laddering pattern of different molecular masses from ˜50 to greater than 500 kDa (FIG. 2A, red arrows), most likely representing monomers, dimers, trimers, tetramers, pentamers, hexamers etc. Weaker bands between these species are most likely 25 kDa folding intermediates that include Fc-halfmners (FIG. 2A, blue arrows). As Cys-309 is present in these mutants (FIG. 2A,B), the ladders most likely arise through disulphide bond formation between the only freely available sulfhydryl in Cys-309 in two adjacent monomers. We reasoned that the loss of the tailpiece glycan in these four N563A mutants allows hydrophobic amino-acid residues (Val-564, Leu-566 and Ile-567) located in the tailpiece to cluster, thereby allowing disulphide bonding at Cys-309.

By running these mutants under reducing conditions, we were able to determine the relative sizes and occupancy of the various glycans attached at each position, showing that the Asn-221- and Asn-563-attached glycans are larger than those attached at Asn-297, and that fully aglycosylated null mutants (N297A/N563A/C575A) are −10 kDa lighter than either hexa-Fc or C575A glycan competent molecules (FIG. 2B). As Cys309 is present in these mutants (FIG. 1, FIG. 2A,B), the ladders may arise through disulfide bond formation between the only freely available sulfhydryl at Cys309 in two adjacent monomers. We reasoned that the loss of the tailpiece glycan in these four N563A mutants allows the hydrophobic amino-acid residues (Val564, Leu566 and Ile567) located in the tailpiece to cluster, thereby permitting disulfide bonding at Cys309.

To test the hypothesis that Cys-309 was indeed responsible for the laddering seen with the N563A deficient mutants, we generated a second panel of C575A mutants in which Cys-309 was mutated to Leu-309 and Leu-310 to His-310 as found in the wild-type IgG1-Fc sequence (FIG. 1B). We also generated the mutant C309L in which the tailpiece Cys575 was still present. This mutant ran similarly to hexa-Fc under non-reducing conditions, albeit with the presence of intermediates (FIG. 2C, blue arrows) that were notably absent in hexa-Fc, showing that Cys-309 stabilizes the quaternary structure in the presence of Cys-575.

Importantly, the loss of Cys-309 also resulted in the loss of the ladders previously seen in Cys-309 competent mutants (FIG. 2, panel C versus panel A), with all the double cysteine mutants now running principally as monomers. The C309L/N297A/C575A mutant runs as four different monomeric species (FIG. 2C) that resolve as two bands under reduction (FIG. 2D), These bands most likely represent glycan variants arising at Asn-563. Given that these variants are absent in the C309L/C575A mutant, we conclude that the presence of Asn-297 glycan also controls glycosylation efficiency at Asn-563. To some extent, the presence of the Asn-221 glycan also limits the occurrence of these Asn-563 glycoforms, as under reduction only a single band is seen in the D221N/C309L/N297A/C575A mutant (FIG. 2D).

Although the panel of double cysteine knockouts run mostly as monomers on SDS-PAGE (FIG. 2C), the double-cysteine knockouts also containing the N563A substitution run as a mixture of monomers and multimers in solution (FIG. 12). Thus, removal of the bulky Asn563 glycan exposes hydrophobic amino acid residues in the tailpiece that facilitate non-covalent interactions in solution that would not otherwise readily occur in the presence of the sugar.

The Asn-297 and Asn-563 Glycans are Critical for the Interactions of Monomers with Glycan Receptors and their Absence can be Compensated by the Presence of Asn-221

To determine which N-linked glycan in the double-cysteine knockout mutants (FIG. 1B) contributes to receptor binding, we investigated their interaction with soluble recombinant glycan receptors by ELISA (FIG. 3 and summarized in Table 1). In stark contrast to the IgG-Fc control, tailpiece monomers in which both Asn-297 and Asn-563 are present (e.g. C309L/C575A) bound all twelve glycan receptors investigated (FIG. 3).

Removal of the glycan at Asn-563, as in C309L/N563A/C575A or C309L/N297A/N563A/C575A mutants, abolished binding to these same twelve receptors, showing that Asn-563 is required for glycan-receptor bind.

Removal of the glycan at Asn-297, as in C309L/N297A/C575A, also abolished binding to all receptors with the exception of Siglec-1. This data shows that the presence of both Asn-563 and Asn-297 are required for glycan receptor binding of the C309L/C575A monomeric mutant.

With the exception of MBL, MMR, and DC-SIGN, binding by the double aglycosylated knockout mutant C309L/N297A/N563A/C575A could be reinstated by the addition of sialylated glycan at Asn-221 (as with D221N/C309L/N297A/N563A/C575A). The Asn221 glycan contributes all the sialylated sugars that are required to explain the marked improvements in binding to other glycan receptors, compared to all equivalent mutants lacking Asn221. This is in agreement with our previous work where we demonstrated in fully cysteine-competent multimers that Asn-221 is >75% terminally sialylated²³, and the presence of this glycan must explain the marked improvements in binding to the other nine glycan receptors that were investigated, compared to all equivalent mutants lacking Asn-221.

Interestingly, the C309L mutant that forms cysteine-linked multimers due to the retention of Cys-575 in the tailpiece (FIG. 2C) was unable to bind to any glycan receptors investigated with the notable exception of CD23 (FIG. 3). Thus, the Asn-563 tailpiece-attached glycans are only available for binding when attached to lower-valency molecules and are most likely buried within multimers that form either through covalent (e.g. throughCys-309) or non-covalent (e.g. C309L/N563A/C575A) bonding.

We then investigated binding of the panel of C575A mutants in which Cys-309 is still present (FIG. 1A), and that we had observed also had a propensity to form dimers and laddered multimers (FIG. 2A). This panel of molecules in which disulphide bonding mediated by Cys-309 could still occur, bound less well to all the glycan receptors investigated (FIG. 4), and as observed with the C309L multimers. With the sole exception of Siglec-1, the presence of the Asn-221 glycan was unable to improve binding, as seen with the panel of true monomers. We conclude that N-glycans at all three attachment sites (Asn-221, Asn-297 and Asn-563) are pre-disposed to binding to glycan-receptors when expressed on true monomers, and that the presence of Asn-221 on its own (as the only glycan) is sufficient to impart this broad specificity of binding, as exemplified by both the D221N/N297A/N563A/C575A and D221N/C309L/N297A/N563A/C575A mutants.

We observed that the aglycosylated mutant N297A/N563A/C575A had a propensity to bind glycan-receptors (FIG. 4). The lack of binding by its counterpart C309L/N297A/N563A/C575A in which Cys309 is absent, suggests that it may be glycan independent and a consequence of increased avidity interactions through multimerization (FIG. 2A).

### Asn-221 Based Monomers Show Enhanced Binding to Low-Affinity FcγRs

Given the remarkable binding to glycan receptors seen with the hyper-glycosylated monomers, we tested the impact that this extra glycosylation site has on binding to the classical FcγRs (FIG. 5 and summarized in Table 2). The presence of Asn-221, for example in the D221N/C309L/N297A/N563A/C575A mutant, imparted improved binding of monomers to FcγRIIB (CD32B) even in the absence of both Asn-297 and Asn-563 when compared to the IgG1-Fc, and controls in which Asn-221 was absent (FIG. 5, for FcγRIIB compare filled symbols versus unfilled symbols). However, the presence of Asn-221 did not improve binding to FcγRIIIA (compare D221N/C309L/N563A/C575A versus C309L/N563A/C575A), although binding of both glycan mutants was significantly better than the IgG1-Fc monomer control (FIG. 5). Improved binding to FcγRI was also observed with these two mutants against the IgG1-Fc control, although no improvements were seen with respect to either FcγRIIA or FcγRIIIB for any of the mutants tested.

We then investigated binding of the multimers formed through Cys-309 (FIGS. 1A and 2A). In multimers, the presence of Asn-221 generally reduced binding to all the FcγRs (FIG. 6 and summarized in Table 2), while binding to the glycan receptors, although lower than that seen with monomers, was retained (FIG. 4). Multimers in which Asn-563 and Cys-575 are both mutated to alanine, as in N563A/C575A, bound very strongly to FcγRI and FcγRIIIA, with improved binding to FcγRIIB when compared to either the hexa-Fc or IgG1-Fc controls (FIG. 6). Finally, aglycosylated multimers (e.g. N297A/N563A/C575A) showed improvements in binding to FcγRI and the inhibitory FcγRIIB receptor (FIG. 6).

### Asn-221-Based Monomers and Multimers Show Reduced Complement Activation

Binding of C1q and activation of the classical complement pathway by complex monomers (FIG. 7) and multimers (FIG. 8) was assessed using ELISA as previously described^{23,30} (summarized in Table 3). With the exception of D221N/C309L/N563A/C575A, all Asn-221-containing monomers bound C1q less well than the IgG1-Fc or Asn-221-deficient controls (FIG. 7A), and all four Asn-221-containing monomers were unable to activate the classical complement pathway to its terminal components (FIG. 7B). These findings were recapitulated with the Cys-309 competent panel of mutants, including mutants shown to form multimers (e.g. D221N/N297A/N563A/C575A against N297A/N563A/C575A). We also identify mutants capable of forming multimers (e.g. C309L and D221N/N563A/C575A) that avidly bound C1q but were unable to fix C5b-9 when compared with hexa-Fc (FIG. 8, panel A vs B).

### Asn221-Based Monomers and Multimers Exhibit Complex Sialylation Patterns

The structure of the N-glycan on the Fc of IgG antibodies has been shown to influence multiple receptor interactions. For example, the interaction of IVIG with glycan receptors has been attributed to direct and/or indirect effects of N-glycan sialic acid on the Fc^{28, 51,48}. Therefore, we investigated the nature of the N-glycans on the two panels of glycosylation- and cysteine-deficient mutants by MALDI-TOF mass spectrometry based glycomic analysis (FIG. 9).

We have previously demonstrated that N-glycans from both IgG1-Fc and clinical IVIG preparations are dominated by biantennary complex N-glycans with 0, 1 or 2 galactose residues². A minority of these complex structures are also mono-sialylated^{2,22}. Representative glycomic data is presented in FIG. 9 for N297A/C575A and D221N/N297A/N563A/C575A.

In both samples the spectra demonstrate a higher level of N-glycan processing with enhanced levels of biantennary galactosylation and sialylation. In addition, larger tri- and tetra-antennary complex N-glycans are also observed which can be fully sialylated (for example peaks at m/z 3776 and 4586). Therefore, the glycomic analysis revealed that both Asn-221 and Asn-575 contained larger, more highly processed N-glycans that are not observed on the IgG1-Fc control. As predicted, no glycans could be detected on the glycosylation-deficient double mutants (N297A/N563A/C575A and C309L/N297A/N563A/C575A).

### The Asn221 Glycan Imparts Enhanced Binding to Influenza Hemagglutinin

To determine if any of the hyper-sialylated Fc mutants possessed biologically useful properties, we investigated their binding to hemagglutinin, a prototypic viral sialic-acid binding ligand (FIG. 10A, 10B). We used clinically available IVIG as a positive control, as IVIG is known to contain high concentrations of IgG antibodies against a diverse range of influenza hemagglutinins⁶⁷.

As expected, IVIG bound strongly to recombinant hemagglutinin from both influenza A and B viruses (FIG. 10A, 10B). With the exception of the aglycosylated mutants (C309L/N297A/N563A/C575A and N297A/N563A/C575A) and the IgG1-Fc control, all the glycan-modified Fc fragments bound recombinant hemagglutinin from both group A and B viruses. Binding was also reflected in the abundance of sialylated N-glycans of the mutant proteins. Mutants containing Asn221 bound more strongly than their equivalents in which Asn221 was absent (FIG. 10A, 10B).

Although binding to native inactivated influenza strain A New Caledonia/20/99virus (H1N1) was poorer than binding to the recombinant hemagglutinins, two mutants (D221N/C309L/N297A/C575A and D221N/C575A) showed superior binding to the native virus compared to either IVIG or their equivalent mutants in which Asn221 was absent (compare C575A with D221N/C575A) (FIG. 10A, 10B). This surprising finding opens the door for use of the molecules of the invention that are glycosylated at the amino terminus, such as the molecules with Asn221 for use in treating or preventing viral infection, such as influenza, particularly when the molecule is also glycosylated at or close to the carboxy terminus, such as Asn 563 or Asn573.

### Asn221-Containing Mutants Inhibit Hemagglutination by Influenza.

To test if the binding to hemagglutinin has any functional relevance we used the World Health Organization (WHO)-based hemagglutination inhibition (HI) protocol to quantify influenza-specific inhibitory titers of the mutants that bound the native virus strongly (FIG. 10C). Both D221N/C309L/N297A/C575A and D221N/C575A prevented hemagglutination by New Caledonia/20/99virus (H1N1) at concentrations as low as 0.1 M and were demonstrably more effective than molar equivalents of either IVIG or anti-H1N1 polyclonal IgG. In contrast, the equivalent molecules that lack Asn221, i.e. C309L/N297A/C575A and C575A, failed to inhibit hemagglutination although partial inhibition was observed with the C575A mutant at the highest concentrations in some experiments (FIG. 10C). Hence, receptor binding of influenza A viruses is competed out only by mutants in which Asn221 is present. That both D221N/C309L/N297A/C575A and D221N/C575A mutants run entirely as monomers by SEC-HPLC (see FIG. 12) implies that the disposition of the glycans, in particular Asn221, are more favorably orientated for binding native virus in monomers than multimers.

TABLE 1

| | Complex | | | | | | | |
|---|---|---|---|---|---|---|---|---|
| | sialylated | | | | | | | |
| | glycans | | | | | | | |
| Glycan receptors | detected | Siglec-1 | Siglec-2 | Siglec-3 | Siglec-4 | CD23 | dectin-1 | dectin-2 |
| C309L/N297A/N563A/C575A | − | − | − | − | − | − | − | − |
| IgG1-Fc | − | − | − | − | − | − | − | − |
| N297A/C575A | + | + | − | − | − | −/+ | − | − |
| D221N/N297A/C575A | +++ | ++ | − | − | − | −/+ | − | − |
| C309L/N297A/C575A | +++ | ++ | − | − | −/+ | − | − | − |
| D221N/C309L/N297A/C575A | +++ | ++ | −/+ | − | +++ | | − | − |
| C575A | −/+ | + | − | − | − | −/+ | − | − |
| D221N/C575A | +++ | ++ | + | − | + | −/+ | − | − |
| C309L/C575A | −/+ | +++ | +++ | +++ | +++ | +++ | ++ | ++ |
| D221N/C309L/C575A | + | ++++ | ++++ | ++++ | ++++ | ++++ | +++ | +++ |
| C309L/N563A/C575A | − | − | − | − | −/+ | − | − | − |
| D221N/C309L/N297A/N563A/C575A | ++ | ++ | +++ | ++ | +++ | + | + | ++ |
| D221N/C309L/N563A/C575A | + | ++ | +++ | + | +++ | + | + | + |
| D221N/N297A/N563A/C575A | +++ | ++ | −/+ | + | + | + | − | −/+ |
| Hexa-Fc | + | ++ | ++ | − | −/+ | + | − | − |
| D221N/N563A/C575A | + | + | − | − | −/+ | − | − | −/+ |
| C309L | ++ | − | − | − | − | + | − | − |
| N297A/N563A/C575A | − | + | + | − | + | − | −/+ | −/+ |
| N563A/C575A | −/+ | ++ | −/+ | − | − | − | −/+ | −/+ |

| Glycan receptors | DC-SIGN | dec-4A | dec-4D | MBL | MMR | DEC-205 |
|---|---|---|---|---|---|---|
| C309L/N297A/N563A/C575A | − | − | − | − | − | − |
| IgG1-Fc | − | − | − | − | − | nd |
| N297A/C575A | − | − | − | − | − | nd |
| D221N/N297A/C575A | − | − | − | − | − | nd |
| C309L/N297A/C575A | − | − | − | − | − | nd |
| D221N/C309L/N297A/C575A | − | − | − | − | − | nd |
| C575A | − | − | − | − | − | nd |
| D221N/C575A | − | − | − | − | − | nd |
| C309L/C575A | + | + | + | + | + | +++ |
| D221N/C309L/C575A | + | ++ | + | +++ | +++ | +++ |
| C309L/N563A/C575A | − | − | − | − | − | nd |
| D221N/C309L/N297A/N563A/C575A | −/+ | + | −/+ | − | − | ++++ |
| D221N/C309L/N563A/C575A | −/+ | + | −/+ | − | − | nd |
| D221N/N297A/N563A/C575A | − | − | + | − | − | nd |
| Hexa-Fc | + | − | − | − | − | + |
| D221N/N563A/C575A | − | − | − | − | − | nd |
| C309L | − | − | − | − | − | − |
| N297A/N563A/C575A | + | − | − | + | ++ | nd |
| N563A/C575A | −/+ | −/+ | − | − | − | nd |

TABLE 2

| | Complex | | | | | |
|---|---|---|---|---|---|---|
| | sialylated | | | | | |
| | glycans | | | | | |
| Fcγ-receptors | detected | Fcγ-RI | Fcγ-RIIA | Fcγ-RIIB | Fcγ-RIIIA | Fcγ-RIIIB |
| C309L/N297A/N563A/C575A | − | − | − | − | − | − |
| IgG1-Fc | − | + | − | − | − | − |
| N297A/C575A | + | − | − | − | − | − |
| D221N/N297A/C575A | +++ | − | − | − | − | − |
| C309L/N297A/C575A | +++ | − | − | − | − | − |
| D221N/C309L/N297A/C575A | +++ | − | − | − | − | − |
| C575A | −/+ | + | − | − | − | − |
| D221N/C575A | +++ | + | − | −/+ | − | − |
| C309L/C575A | −/+ | + | − | − | − | − |
| D221N/C309L/C575A | + | + | − | + | − | − |
| C309L/N563A/C575A | − | ++ | − | − | +++ | − |
| D221N/C309L/N297A/N563A/C575A | ++ | + | − | ++ | −/+ | − |
| D221N/C309L/N563A/C575A | + | ++ | − | +++ | ++ | − |
| D221N/N297A/N563A/C575A | +++ | − | − | − | − | − |
| Hexa-Fc | + | ++ | − | −/+ | + | − |
| D221N/N563A/C575A | + | ++ | − | − | ++ | − |
| C309L | ++ | ++ | − | − | +++ | − |
| N297A/N563A/C575A | − | ++ | − | +++ | − | − |
| N563A/C575A | −/+ | ++++ | − | + | ++++ | − |

TABLE 3

| | | | | Binds | | | |
|---|---|---|---|---|---|---|---|
| | | | | Native | | | Inhibits |
| | Complex | | | Influenza | Binds | Binds | Influenza |
| | sialylated | | | virus | Recombinant | Recombinant | Virus |
| | glycans | | | (Caledonia | HA (Shantou | HA (Florida | (Caledonia |
| | detected | C1q | C5b-9 | A/H1N1) | A/H3N8) | B) | A/H1N1) |
| C309L/N297A/N563A/C575A | − | + | + | − | − | − | No |
| IgG1-Fc | − | + | + | − | − | − | No |
| N297A/C575A | + | − | − | − | −/+ | − | n.d. |
| D221N/N297A/C575A | +++ | − | − | − | ++ | + | n.d. |
| C309L/N297A/C575A | +++ | − | − | − | +++ | ++ | No |
| D221N/C309L/N297A/C575A | +++ | − | − | ++++ | +++ | +++ | Yes |
| C575A | −/+ | −/+ | −/+ | − | ++ | + | No |
| D221N/C575A | +++ | − | − | ++++ | +++ | ++ | Yes |
| C309L/C575A | −/+ | + | −/+ | − | + | − | No |
| D221N/C309L/C575A | + | −/+ | −/+ | + | + | − | n.d. |
| C309L/N563A/C575A | − | ++ | + | − | + | − | n.d. |
| D221N/C309L/N297A/N563A/C575A | ++ | + | − | + | ++ | + | n.d. |
| D221N/C309L/N563A/C575A | + | ++ | − | + | ++ | + | n.d. |
| D221N/N297A/N563A/C575A | +++ | −/+ | − | − | + | −/+ | n.d. |
| Hexa-Fc | + | +++ | + | + | + | − | n.d. |
| D221N/N563A/C575A | + | +++ | − | − | ++ | + | n.d. |
| C309L | ++ | +++ | − | − | ++ | −/+ | n.d. |
| N297A/N563A/C575A | − | + | + | − | − | − | No |
| N563A/C575A | −/+ | ++ | −/+ | − | − | − | |

TABLE 4
mutations in the certain of the exemplified proteins of the invention

| SEQ | | |
|---|---|---|
| ID | | |
| NO. | Also referred to as | Changes compared to SEQ ID NO: 1 |
| 1 | Hexa-Fc or HexaGard | None |
| 2 | C309L/C575A | Cysteine at residue 89 substituted with |
| | | leucine; leucine at residue 90 substituted |
| | | with histidine; and cysteine at residue 248 |
| | | substituted with alanine |
| 3 | D221N/C309L/C575A | Aspartic acid at residue 1 substituted with |
| | | asparagine; cysteine at residue 89 substituted |
| | | with leucine; leucine at residue 90 |
| | | substituted with histidine; and, cysteine at |
| | | residue 248 substituted with alanine |
| 4 | C309L/N563A/C575A | Cysteine at residue 89 substituted with |
| | | leucine; leucine at residue 90 substituted |
| | | with histidine; asparagine at residue 236 |
| | | substituted with alanine; and cysteine at |
| | | residue 248 substituted with alanine |
| 5 | D221N/C309L/N297A/N563A/C575A | Aspartic acid at residue 1 substituted with |
| | | asparagine; cysteine at residue 89 substituted |
| | | with leucine; leucine at residue 90 |
| | | substituted with histidine; asparagine at |
| | | residue 77 substituted with alanine; |
| | | asparagine at residue 236 substituted with |
| | | alanine; and, cysteine at residue 248 |
| | | substituted with alanine |
| 6 | D221N/C309L/N563A/C575A | Aspartic acid at residue 1 substituted with |
| | | asparagine; cysteine at residue 89 substituted |
| | | with leucine; leucine at residue 90 |
| | | substituted with histidine; asparagine at |
| | | residue 236 substituted with alanine; and, |
| | | cysteine at residue 248 substituted with |
| | | alanine |
| 7 | D221N/C309L/N297A/C575A | Aspartic acid at residue 1 substituted with |
| | | asparagine; cysteine at residue 89 substituted |
| | | with leucine; leucine at residue 90 |
| | | substituted with histidine; asparagine at |
| | | residue 77 substituted with alanine; and, |
| | | cysteine at residue 248 substituted with |
| | | alanine |
| 8 | C309L/N297A/C575A | Cysteine at residue 89 substituted with |
| | | leucine; leucine at residue 90 substituted |
| | | with histidine; asparagine at residue 77 |
| | | substituted with alanine; and, cysteine at |
| | | residue 248 substituted with alanine |
| 9 | D221N/N297A/N563A/C575A | Aspartic acid at residue 1 substituted with |
| | | asparagine; asparagine at residue 77 |
| | | substituted with alanine; asparagine at residue |
| | | 236 substituted with alanine; and, cysteine at |
| | | residue 248 substituted with alanine |
| 10 | N297A/N563A/C575A | Asparagine at residue 77 substituted with |
| | | alanine; asparagine at residue 236 substituted |
| | | with alanine; and, cysteine at residue 248 |
| | | substituted with alanine |
| 11 | C309L | cysteine at residue 89 substituted with leucine; |
| | | leucine at residue 90 substituted with |
| | | histidine. |
| 55 | C309L/N297A/N563A/C575A | Cysteine at residue 89 substituted with |
| | | leucine; asparagine at residue 77 substituted |
| | | with alanine; leucine at residue 90 |
| | | substituted with histidine; asparagine at |
| | | residue 236 substituted with alanine and, |
| | | cysteine at residue 248 substituted with |
| | | alanine |
| 57 | N297A/C575A | asparagine at residue 77 substituted with |
| | | alanine; and, cysteine at residue 248 |
| | | substituted with alanine |
| 59 | D221N/C575A | Aspartic acid at residue 1 substituted with |
| | | asparagine; and, cysteine at residue 248 |
| | | substituted with alanine |

Generating commercial multimeric Fcs raises significant bioprocessing and safety issues that are not found with monomeric Fc production. For example, high mannose type glycans found in hexa-Fc have been shown to increase IgG clearance rates due to cellular uptake via the mannose receptor. Recombinant monomeric Fcs developed here that are devoid of oligomannose, and yet show improved binding to selected glycan receptors may therefore have significant therapeutic potential, for example as replacements for IVIG. Furthermore, given the known effects of Fc-sialylation in reducing IgG antibody-dependent cellular cytotoxicity activity, it may also be possible to use the mutations to develop therapeutic antibodies with modified effector functions.

Multimeric Fcs (such as those with SEQ ID NO: 9-11) may nonetheless be useful, for example when delivering antigens in vaccines or as high avidity receptor blockers. Many pathogens rely on glycans to infect host cells, and differentially glycosylated Fc-multimers may be useful inhibitors of infection. One immediate application for our hypersialylated molecules may be to block Siglec-1 dependent trans-infection of lymphocytes by retroviruses, including HIV and human T-cell leukaemia viruses. We anticipate that expression of these mutants in human cell lines e.g. HEK, will bestow hypersialylated molecules with α2,6 linkages with improved binding to α2,6-dependent receptors like Siglec-2 that are implicated in IVIG efficacy. Such receptor mimicry strategies need to overcome the high avidity of the natural receptor generated by the sum of the multiple low-affinity glycan binding sites that may now be achievable with the D221N series of hypersialylated multimers. Thus by adding or removing glycosylation and disulphide bonding sites within hexa-Fc, new portfolios of effector functions can be generated.

### DISCUSSION

Many groups have postulated that multivalent Fc constructs may have potential for the treatment of immune conditions involving pathogenic antibodies^{2,29,31,32} and a recent study has shown that hexameric-Fcs can block FcγRs leading to their down-modulation and prolonged disruption of FcγR effector functions both in vitro and in vivo³³. Hexameric-Fcs have also recently been shown to inhibit platelet phagocytosis in mouse models of ITP^{33,34}.

Although hexameric-Fcs may provide exciting new treatment approaches to control autoimmune disease, their beneficial effects must be carefully balanced with the acute risk of pro-inflammatory responses observed upon FcγR cross-linking, and the increased risk from infection or cancers, due to long term immune-suppression. These potential drawbacks with multimeric-Fcs led us to investigate if complex monomers may be developed that retain the advantages of multimers, e.g. high-avidity binding to low-affinity receptors but without these potential risks.

Although Fc-engineering by mutagenesis and/or direct modifications to the Asn-297 glycan have yielded modified affinity and/or selectivity for FcγRs^{1,17,35-40}, interactions with glycan receptors have largely been ignored despite a large body of literature demonstrating their importance in controlling unwanted inflammation⁴¹⁻⁴⁴. However, such approaches that show enhanced receptor interactions via mutations introduced into full-length IgG molecules⁴⁵⁻⁴⁷, may not necessarily be predictive a priori in the context of either Fc-multimers or their Fc-fragments^{23,33}.

Furthermore, reported Fc mutations or glycan modifications have mostly focused on the conserved Asn-297 glycan that is largely buried within the Fc^{4,16-19}, and thus monomeric IgG1 is unable to interact with a broader range of glycan receptors. Although Siglec-2⁴⁸, DC-SIGN^{2,49,50}, DCIR⁵¹, and FcRL5^{2,52} have all recently been shown to be ligands for IVIG, these interactions may also stem from specific Fab-mediated binding⁵³. Therefore, glycosylation of intact IgG is known to be critically important, but the relative contribution of the Fc and Fab domains, together with the identity of the salient receptors involved in IVIG efficacy, remains controversial.

We took an alternative approach to glycan modification by introducing, in various combinations, two additional N-linked glycosylation sites (Asn-221 and Asn-563) into multimeric IgG1-Fcs²³. To investigate the effects of additional glycosylation on monomer function, these glycan-modified multimers (hexa-Fcs) were further mutated to remove one or both the cysteine residues (Cys-309 and Cys-575) that are required for inter-disulphide bond formation between individual Fc moieties (FIG. 1B). This approach yielded complex glycosylated monomers (FIG. 2C), including the D221N/C309L/C575A mutant with all three glycans attached, that showed improved binding to FcγRIIB, DC-SIGN, and DCIR; these receptors being implicated in the efficacy of IVIG (Table 1)^{7,16,19,54,55}.

The D221N/C309L/C575A tri-glycan mutant bound more strongly and broadly to all the glycan receptors investigated, including receptors recently implicated in IVIG efficacy e.g., CD23⁵⁶, CD22⁴⁸, and DCIR (clec4a)⁵¹, when compared to mono-glycosylated (e.g. IgG1-Fc) or non-glycosylated (C309L/N297A/N563A/C575A) controls (FIG. 3 and Table 1).

The observed binding to CD22 was particularly surprising as this receptor prefers α-2,6 linked neuraminic acid and not α-2,3 linkages attached by CHO-K1 cells, although proximity labelling experiments have recently shown that glycan-independent interactions of CD22/Siglec-2 with immunoglobulin in the B-cell receptor is possible⁶⁸. We also observed marked binding of D221N/C309L/C575A to dectins (FIG. 3), receptors that more typically recognize β-1,3-glucans expressed by fungal pathogens⁶⁹. Although dectin-1 is known to bind variably glycosylated human tetraspanins CD37 and CD63⁷⁰, the anti-inflammatory activity of IgG1 immune complexes may be mediated by Fc galactosylation and associations with dectin-1 and FcγRIIB⁷¹.

The insertion of multiple glycan sites into the body of the Fc, in particular at Asn-221, has also enabled new receptor interactions that are not possible with Asn-297-directed approaches. For example, we generated the di-glycan D221N/C309L/N297A/C575A mutant that displayed marked binding to Siglec-1 and Siglec-4 (MAG), both receptors being clinically implicated in the control of neuropathy^{24,25} This mutant showed no observable binding to either FcγRs or complement proteins (FIG. 5 and Tables 2,3).

As glycan-mediated binding is essential for the influenza virus to infect cells of the respiratory tract, mutations in hemagglutinin that lead to the loss of receptor binding are unlikely to survive neutralizing antibodies induced during an immune response. Modelling of the D221N/C575A mutant shows that the distance from the N-terminal to the C-terminal tips of the Fc is ˜60Δ, the same approximate distance found between the sialic-acid binding domains within the hemagglutinin trimer⁷².

Alternative anti-influenza therapeutic strategies are urgently needed.

The use of IVIG during the 2009 and 1918 pandemics reduced mortality from influenza by 26% and 50% respectively^{73, 74}, and a recent randomized, placebo-controlled study suggests these figures may be improved by enhancing influenza-specific antibodies in IVIG (Flu-IVIG) preparations⁷⁵. As Flu-IVIG is manufactured in advance of future epidemics, there could be modest or no neutralizing activity against emerging strains. Combinations of Flu-IVIG with Fc sialic-acid binding domain blockers may enhance the efficacy of Flu-IVIG preparations. Neither the D221N/C575A nor D221N/C309L/N297A/C575A mutants that inhibited hemagglutination so effectively (FIG. 10C), bind FcγRIIIA (FIGS. 5, 6 and Table 1), and would thus not be expected to interfere with FcγRIIIA dependent ADCC toward influenza-infected cells by neutralizing IgG present in Flu-IVIG.

The molecules of the invention that bind haemagglutinin, such as D221N/C575A and D221N/C309L/N297A/C575A, are thus predicted to be useful in impeding viral infection and may be used alone or in conjunction with IVIG, such as Flu-IVIG. Such molecules also run as true monomers (as determined by SEC-HPLC) which makes them particularly suited for therapeutic use as (i) manufacturability and processing will be easier as there is only one form produced; and (ii) homogeneous preparations are possible ensuring that reproducibe product is available for administration.

Intranasal delivery of Fc fragments may also be therapeutically beneficial as Fc-fused interleukin-7 can provide long-lasting prophylaxis against lethal influenza virus after intranasal delivery⁷⁶. We have previously shown that Fc multimers can bind the neonatal Fc-receptor (FcRn)⁷⁷. Thus, binding to the neonatal FcRn may act to increase the residence time of Fc blockers delivered to the lung^{78, 79}. A potential drawback to the hyper-sialylation approach may be the susceptibility of Fc glycans to viral neuraminidase, although metabolic oligosaccharide engineering with alkyne sialic acids could confer neuraminidase-resistant Fc blockers⁸⁰.

In another example, multiple mutants were shown to bind DEC-205 (FIG. 3,4 and Table 1) which is the major endocytic receptor expressed by dendritic cells, which suggests that these constructs may be useful for the targeted delivery of antigens in vaccines. Current approaches to deliver antigen to DEC-205 rely on DEC-205-specific delivery, often with antigens fused to anti-DEC-205 mAbs⁵⁷⁻⁵⁹ whereas approaches that target multiple DC receptors, including DEC-205, may make for more effective antigen delivery, e.g. in vaccines.

To be useful in vaccines, an antigen must cluster through the binding of multiple Fc regions in near-neighbour interactions with multiple low-affinity FcγRs⁸¹, and in particular FcγRIIA, FcγRIIB and FcγRIIIA⁸¹⁻⁸³. As described above we generated multimers with differential binding to either FcγRIIB (e.g. N297A/N563A/C575A), FcγRIIIA (e.g. C309L and D221N/N563A/C575A) or with a capability to bind both FcγRIIB and FcγRIIIA (e.g. N563A/C575A). Multimers formed by the N563A/C575A mutant may be particularly relevant, as these were also able to bind type 2 glycan receptors and activate the complement cascade, both implicated in the efficacy of vaccines²⁹.

As summarized in Table 1-3, we have identified: i) mutant Fc molecules that are capable of binding C1q and activating complement, but that show little or no detectable interaction with either FcγRs or glycan receptors (e.g. C309L/N297A/N563A/C575A); ii) molecules with enhanced activation of complement, improved binding to FcγRs and little engagement of glycan receptors (e.g. C309L/N563A/C575A); iii) molecules with enhanced binding to C1q but little C5b-9 deposition that retain interaction with both Fcγ- and glycan-receptors (e.g. D221N/C309L/N563A/C575A); and finally iv) molecules with enhanced binding to a subset of sialic-acid-dependent glycan-receptors, in particular Siglec-1 and Siglec-4 and haemagglutinin, with little or no interaction with either FcγRs or complement (e.g. D221N/C309L/N297A/C575A). Each of these molecules lack a cysteine residue at positions 309 and 575. Consequently, by adding or removing glycosylation and/or disulfide-bonding sites within our original hexameric Fc platform^{2, 29, 23}, new repertoires of desired binding attributes can be made.

We also describe multimers created from hexameric-Fc, with potentially more desirable properties that may mitigate against unwanted FcγR-dependent pro-inflammation. For example, the D221N/N297A/N563A/C575A multimer was unable to bind FcγRs or activate complement, yet showed enhanced binding to glycan-receptors, including Siglec-1 (Table 1-3).

We also developed multimers that might be useful as vaccine delivery vehicles (e.g. in Fc-antigen-fusions) or as high-avidity receptor blockers. To be useful in vaccines, an antigen must cluster through the binding of multiple Fc regions in near-neighbour interactions with multiple low-affinity FcγRs 6⁰, and in particular FcγRIIA, FcγRIIB and FcγRIIIA⁶⁰-6² As described with monomers, we generated multimers with differential binding to either FcγRIIB (e.g. N297A/N563A/C575A), FcγRIIIA (e.g. C309L and D221N/N563A/C575A) or with a capability to bind both FcγRIIB and FcγRIIIA (e.g. N563A/C575A). Multimers formed by the N563A/C575A mutant may be particularly relevant, as these were also able to bind type 2 glycan receptors and activate the complement cascade, both implicated in the efficacy of vaccines²⁹.

Histidine at 310 is essential for interactions with the neonatal FcRn required for maintaining long half-lives in vivo and for moving IgG from blood into secretions e.g. lungs. The double-cysteine mutants have His at 310, but the C575A only mutants do not. However, the inventors have shown by replacing the Leu310 in hexa-Fc with His you can reinstate binding to FcRn⁷⁷. Molecules with 310His have longer half-lives.

IgG1-based Fc-multimers (termed Stradomers) have recently been described for the treatment of diverse complement-mediated diseases^{31,63,64}. Stradomers have been shown to sequester C1q thus preventing the activation of the classical pathway at the site of inflammation⁶³. In contrast to tailpiece-driven multimerization, these molecules are formed through cysteine residues provided by C-terminal fusions of the human IgG2 hinge to the Fc of IgG1³¹. The sequence of the IgG2 hinge, allows for multimerization via one of four possible cysteine residues, but would not impart binding to glycan receptors as, in contrast to the molecules described herein, N-linked attachment sites are absent from the IgG2 hinge.

Additional hydrophobic residues provided within the tailpiece also allow for non-covalent interactions between monomers that would not be predicted from the amino-acid residues contained within the IgG2 hinge, as exemplified by C309L/N297A/N563A/C575A. These hydrophobic residues have been shown in the absence of the tailpiece Cys-575, to allow IgG1-Fc monomers to assemble into highly potent multimers when they reach their target site (see WO2017/005767). Mutants that possess N563A have a tendency to multimerise in solution, though on sodium dodecyl sulphate (SDS) gels they run as monomers as the non-covalent interactions that promote multimerization are disrupted by SDS.

Here we show that mutants such as. D221N/C309L/N563A/C575A) and D221N/N563A/C575A and C309L can be generated that bind C1q avidly without downstream deposition of the C5b-9 membrane-attack complex. Removal of both Asn-297 and Asn-563, as in the C309L/N297A/N563A/C575A mutant, additionally generates monomers that are unable to interact with either FcγRs or glycan-receptors yet retain the ability to bind C1q. Such molecules may therefore be useful in the treatment of complement-mediated illnesses. Thus, by adding or removing glycosylation and disulphide-bonding sites within the hexameric-Fcs, new repertoires of desired binding attributes can be made.

### REFERENCES

1. Dalziel M, Crispin M, Scanlan C N, Zitzmann N, Dwek R A. Emerging principles for the therapeutic exploitation of glycosylation. Science (80-) [Internet] 2014; 343:1235681,1 1235681,8. Available from: http://www.ncbi.nlm.nih.gov/pubmed/24385630
2. Czajkowsky D M, Andersen J T, Fuchs A, Wilson T J, Mekhaiel D, Colonna M, He J, Shao Z, Mitchell D a., Wu G, et al. Developing the IVIG biomimetic, Hexa-Fc, for drug and vaccine applications. Sci Rep [Internet] 2015; 5:9526. Available from: http://www.nature.com/doifinder/10.1038/srep09526
3. Shields R L, Lai J, Keck R, O'Connell L Y, Hong K, Gloria Meng Y, Weikert S H A, Presta L G. Lack of fucose on human IgG1 N-linked oligosaccharide improves binding to human FcγRIII and antibody-dependent cellular toxicity. J Biol Chem 2002; 277:26733-40.
4. Lux A, Yu X, Scanlan C N, Nimmerjahn F. Impact of Immune Complex Size and Glycosylation on IgG Binding to Human FcγRs. J Immunol 2013; 190:4315-23.
5. Debre M, Bonnet M C, Fridman W H, Carosella E, Philippe N, Reinert P, Vilmer E, Kaplan C, Teillaud J L, Griscelli C. Infusion of Fc gamma fragments for treatment of children with acute immune thrombocytopenic purpura. Lancet [Internet] 1993; 342:945-9. Available from: file://o/Referenzmanager/Immunglobulin.PDFs/Debre 1993.pdf
6. Anthony R M, Nimmerjahn F, Ashline D J, Reinhold V N, Paulson J C, Ravetch J V. Recapitulation of IVIG Anti-Inflammatory Activity with a Recombinant IgG Fc. Science (80-) [Internet] 2008; 320:373-6. Available from: http://www.sciencemag.org/content/320/5874/373.abstract %5Cnhttp://www.sciencemag.org/content/320/5874/373.full.pdf
7. Anthony R M, Kobayashi T, Wermeling F, Ravetch J V. Intravenous gammaglobulin suppresses inflammation through a novel T(H)2 pathway: Commentary. Nature 2011; 475:110-3.
8. Liu L. Antibody glycosylation and its impact on the pharmacokinetics and pharmacodynamics of monoclonal antibodies and Fc-fusion proteins. J. Pharm. Sci.2015; 104:1866-84.
9. Li H, Sethuraman N, Stadheim T A, Zha D, Prinz B, Ballew N, Bobrowicz P, Choi B-K, Cook W J, Cukan M, et al. Optimization of humanized IgGs in glycoengineered Pichia pastoris. Nat Biotechnol [Internet] 2006; 24:210-5. Available from: http://www.ncbi.nlm.nih.gov/pubmed/16429149
10. Pepinsky R B, Shao Z, Ji B, Wang Q, Meng G, Walus L, Lee X, Hu Y, Graff C, Garber E, et al. Exposure levels of anti-LINGO-1 Li81 antibody in the central nervous system and dose-efficacy relationships in rat spinal cord remyelination models after systemic administration. J Pharmacol Exp Ther 2011; 339:519-29.
11. St-Amour I, Pare I, Alata W, Coulombe K, Ringuette-Goulet C, Drouin-Ouellet J, Vandal M, Soulet D, Bazin R, Calon F. Brain bioavailability of human intravenous immunoglobulin and its transport through the murine blood-brain barrier. J Cereb Blood Flow Metab [Internet] 2013; 33:1983-92. Available from: http://www.ncbi.nlm.nih.gov/pubmed/24045402
12. Hawkins C P, Munro P M G, Mackenzie F, Kesselring J, Tofts P S, Boulay EPGH Du, Landon D N, Mcdonald W I. Duration and selectivity of blood-brain barrier breakdown in chronic relapsing experimental allergic encephalomyelitis studied by gadolinium-dtpa and protein markers. Brain 1990; 113:365-78.
13. Xu J, Zhao L, Zhang Y, Guo Q, Chen H. CD16 and CD32 Gene Polymorphisms May Contribute to Risk of Idiopathic Thrombocytopenic Purpura. Med Sci Monit 2016; 22:2086-96.
14. Zhang G, Massaad C A, Gao T, Pillai L, Bogdanova N, Ghauri S, Sheikh K A. Sialylated intravenous immunoglobulin suppress anti-ganglioside antibody mediated nerve injury. Exp Neurol [Internet] 2016; 282:49-55. Available from: http://www.sciencedirect.com/science/article/pii/S0014488616301388
15. Finke J M, Ayres K R, Brisbin R P, Hill H A, Wing E E, Banks W A. Antibody blood-brain barrier efflux is modulated by glycan modification. Biochim Biophys Acta—Gen Subj 2017; 1861:2228-39.
16. Washburn N, Schwab I, Ortiz D, Bhatnagar N, Lansing J C, Medeiros A, Tyler S, Mekala D, Cochran E, Sarvaiya H, et al. Controlled tetra-Fc sialylation of IVIg results in a drug candidate with consistent enhanced anti-inflammatory activity. Proc Natl Acad Sci USA [Internet] 2015; 112:201422481. Available from: http://www.pnas.org/lookup/doi/10.1073/pnas.1422481112%5Cnhttp://www.ncbi.nlm.nih.g ov/pubmed/257338810%5Cnhttp://www.pubmedcentral.nih.gov/articlerender.fcgi?artid=PM C4371931
17. Yu X, Baruah K, Harvey D J, Vasiljevic S, Alonzi D S, Song B D, Higgins M K, Bowden T A, Scanlan C N, Crispin M. Engineering hydrophobic protein-carbohydrate interactions to fine-tune monoclonal antibodies. J Am Chem Soc 2013; 135:9723-32.
18. Dekkers G, Plomp R, Koeleman C A M, Visser R, von Horsten H H, Sandig V, Rispens T, Wuhrer M, Vidarsson G. Multi-level glyco-engineering techniques to generate IgG with defined Fc-glycans. Sci Rep [Internet] 2016; 6:36964. Available from: http://dx.doi.org/10.1038/srep36964% o5Cnhttp://www.ncbi.nlm.nih.gov/pubmed/27872474
19. Pagan J D, Kitaoka M, Anthony R M. Engineered Sialylation of Pathogenic Antibodies In Vivo Attenuates Autoimmune Disease. Cell [Internet] 2017; 1-14. Available from: http://linkinghub.elsevier.com/retrieve/pii/S0092867417314319
20. Baldo B A. Chimeric Fusion Proteins Used for Therapy: Indications, Mechanisms, and Safety. Drug Saf2015; 38:455-79.
21. Boesch A W, Brown E P, Cheng H D, Ofori M O, Normandin E, Nigrovic P A, Alter G, Ackerman M E. Highly parallel characterization of IgG Fc binding interactions. MAbs 2014; 6:915-27.
22. Crispin M, Yu X, Bowden T a. Crystal structure of sialylated IgG Fc: Implications for the mechanism of intravenous immunoglobulin therapy. Proc Natl Acad Sci. 110: 3544-3546, 2013
23. Blundell P A, Le N P L, Allen J, Watanabe Y, Pleass R J. Engineering the fragment crystallizable (Fc) region of human IgG1 multimers and monomers to fine-tune interactions with sialic acid-dependent receptors. J Biol Chem 292:12994-13007, 2017
24. Zhang G, Massaad C A, Gao T, Pillai L, Bogdanova N, Ghauri S, Sheikh K A. Sialylated intravenous immunoglobulin suppress anti-ganglioside antibody mediated nerve injury. Exp Neurol 2016; 282:49-55.
25. Wong A H Y, Fukami Y, Sudo M, Kokubun N, Hamada S, Yuki N. Sialylated IgG-Fc: a novel biomarker of chronic inflammatory demyelinating polyneuropathy. J Neurol Neurosurg Psychiatry [Internet] 2016; 87:275-9. Available from: http://jnnp.bmj.com/cgi/doi/10.1136/jnnp-2014-309964%5Cnhttp://www.ncbi.nlm.nih.gov/pubmed/25814494
26. Subedi G P, Hanson Q M, Barb A W. Restricted Motion of the Conserved Immunoglobulin G1 N-Glycan Is Essential for Efficient FcγRIIIa Binding. Structure [Internet] 2014; 22:1478-88. Available from: http://www.sciencedirect.com/science/article/pii/S0969212614002457
27. Frank M, Walker R C, Lanzilotta W N, Prestegard J H, Barb A W. Immunoglobulin G1 Fc domain motions: Implications for Fc engineering. J Mol Biol 2014; 426:1799-811.
28. Pincetic A, Bournazos S, DiLillo D J, Maamary J, Wang T T, Dahan R, Fiebiger B-M, Ravetch J V. Type I and type II Fc receptors regulate innate and adaptive immunity. Nat Immunol [Internet] 2014; 15:707-16. Available from: http://www.nature.com/doifinder/10.1038/ni.2939%05Cnhttp://www.ncbi.nlm.nih.gov/pubm ed/25045879
29. Czajkowsky D M, Hu J, Shao Z, Pleass R J. Fc-fusion proteins: New developments and future perspectives. EMBO Mol. Med.2012; 4:1015-28.
30. Mekhaiel DNA, Czajkowsky D M, Andersen J T, Shi J, El-Faham M, Doenhoff M, McIntosh R S, Sandlie I, He J, Hu J, et al. Polymeric human Fc-fusion proteins with modified effector functions. Sci Rep [Internet] 2011; 1:1-11. Available from: http://www.nature.com/articles/srep00124
31. Jain A, Olsen H S, Vyzasatya R, Burch E, Sakoda Y, Mdrigeon E Y, Cai L, Lu C, Tan M, Tamada K, et al. Fully recombinant IgG2a Fc multimers (stradomers) effectively treat collagen-induced arthritis and prevent idiopathic thrombocytopenic purpura in mice. Arthritis Res Ther [Internet] 2012; 14:R192. Available from: http://www.pubmedcentral.nih.gov/articlerender.fcgi?artid=3580588&tool=pmcentrez&ren dertype=abstract
32. Ortiz D F, Lansing J C, Rutitzky L, Kurtagic E, Prod T, Choudhury A, Washburn N, Bhatnagar N, Beneduce C, Holte K, et al. Elucidating the interplay between IgG-Fc valency and Fc g R activation for the design of immune complex inhibitors. 2016; 158.
33. Qureshi O S, Rowley T F, Junker F, Peters S J, Crilly S, Compson J, Eddleston A, Bjrkelund H, Greenslade K, Parkinson M, et al. Multivalent Fcγ-receptor engagement by a hexameric Fc-fusion protein triggers Fcγ-receptor internalisation and modulation of Fcγ-receptor functions. Sci Rep [Internet] 2017; 7:17049. Available from: http://www.nature.com/articles/s41598-017-17255-8
34. Lewis, B J., Spirig, R., Kasermann, F., Branch D. No Title. In: 27th Regional Congress of the International Society of Blood Transfusion. Copenhagen: Embase Vox Sanguinis; 2017. page 21.
35. Lund J, Pound J D, Jones P T, Duncan A R, Bentley T, Goodall M, Levine B A, Jefferis R, Winte G. Multiple binding sites on the CH2 domain of IgG for mouse Fc??R11. Mol Immunol 1992; 29:53-9.
36. Lund J, Takahashi N, Pound J D, Goodall M, Jefferis R. Multiple Interactions of IgG with Its Core Oligosaccharide Can Modulate Recognition by Complement and Human Fcγ Receptor I and Influence the Synthesis of Its Oligosaccharide Chains. J Immunol 1996; 157:4963-9.
37. Jefferis R, Lund J, Pound J D. IgG-Fc-mediated effector functions: Molecular definition of interaction sites for effector ligands and the role of glycosylation. Immunol. Rev.1998; 163:59-76.
38. Houde D, Peng Y, Berkowitz S A, Engen J R. Post-translational modifications differentially affect IgG1 conformation and receptor binding. Mol Cell Proteomics [Internet] 2010; 9:1716-28. Available from: http://www.mcponline.org/content/9/8/1716.full.pdf
39. Woof J M, Burton D R. Human antibody-Fc receptor interactions illuminated by crystal structures. Nat Rev Immunol [Internet] 2004 [cited 2015 Feb. 18]; 4:89-99. Available from: http://www.ncbi.nlm.nih.gov/pubmed/15040582
40. Saxena A, Wu D. Advances in therapeutic Fc engineering—modulation of IgG-associated effector functions and serum half-life. Front. Immunol.2016; 7.
41. MacAuley M S, Crocker P R, Paulson J C. Siglec-mediated regulation of immune cell function in disease. Nat. Rev. Immunol.2014; 14:653-66.
42. Bochner B S, Zimmermann N. Role of siglecs and related glycan-binding proteins in immune responses and immunoregulation. J. Allergy Clin. Immunol.2015; 135:598-608.
43. Mahajan V S, Pillai S. Sialic acids and autoimmune disease. Immunol Rev 2016; 269:145-61.
44. Bull C, Heise T, Adema G J, Boltje T J. Sialic Acid Mimetics to Target the Sialic Acid-Siglec Axis. Trends Biochem. Sci.2016; 41:519-31.
45. Shields R L, Lai J, Keck R, O'Connell L Y, Hong K, Meng Y G, Weikert S H A, Presta L G. Lack of Fucose on Human IgG1 N-Linked Oligosaccharide Improves Binding to Human FcγRIII and Antibody-dependent Cellular Toxicity. J Biol Chem 2002; 277:26733-40.
46. Shields R L, Namenuk A K, Hong K, Meng Y G, Rae J, Briggs J, Xie D, Lai J, Stadlen A, Li B, et al. High Resolution Mapping of the Binding Site on Human IgG1 for FcγRI, FcγRII, FcγRIII, and FcRn and Design of IgG1 Variants with Improved Binding to the FcγR. J Biol Chem 2001; 276:6591-604.
47. Moore G L, Chen H, Karki S, Lazar G A. Engineered Fc variant antibodies with enhanced ability to recruit complement and mediate effector functions. MAbs 2010; 2:181-9.
48. Seite J F, Cornec D, Renaudineau Y, Youinou P, Mageed R A, Hillion S. IVIg modulates BCR-signaling through CD22 and promotes apoptosis in mature human B lymphocytes. Blood [Internet] 2010; 116:doi:10.1182/blood-2009-12-261461. Available from: file://o/Referenzmanager/Immunglobulin.PDFs/Seite2010.pdf %/5Cnhttp://bloodjournal.hem atologylibrary.org/content/early/2010/06/01/blood-2009-12-261461.full.pdf
49. Fiebiger B M, Maamary J, Pincetic A, Ravetch J V. Protection in antibody- and T cell-mediated autoimmune diseases by antiinflammatory IgG Fcs requires type II FcRs. Proc Natl Acad Sci [Internet] 2015; 201505292. Available from: http://www.pnas.org/lookup/doi/10.1073/pnas.1505292112
50. Schwab I, Biburger M, Kronke G, Schett G, Nimmerjahn F. IVIg-mediated amelioration of ITP in mice is dependent on sialic acid and SIGNR1. Eur J Immunol 2012; 42:826-30.
51. Massoud A H, Yona M, Xue D, Chouiali F, Alturaihi H, Ablona A, Mourad W, Piccirillo C A, Mazer B D. Dendritic cell immunoreceptor: A novel receptor for intravenous immunoglobulin mediates induction of regulatory T cells. J Allergy Clin Immunol 2014; 133.
52. Franco A, Damdinsuren B, Ise T, Dement-Brown J, Li H, Nagata S, Tolnay M. Human Fc Receptor-Like 5 Binds Intact IgG via Mechanisms Distinct from Those of Fc Receptors. J Immunol [Internet] 2013; 190:5739-46. Available from: http://www.jimmunol.org/cgi/doi/10.4049/jimmunol.1202860
53. van de Bovenkamp F S, Hafkenscheid L, Rispens T, Rombouts Y. The Emerging Importance of IgG Fab Glycosylation in Immunity. J Immunol [Internet] 2016; 196:1435-41. Available from: http://www.jimmunol.org/cgi/doi/10.4049/jimmunol.1502136%5Cnhttp://www.ncbi.nlm.ni h.gov/pubmed/26851295
54. Kaneko Y, Nimmerjahn F, Ravetch J V. Anti-inflammatory activity of immunoglobulin G resulting from Fc sialylation. Science (80-) [Internet] 2006; 313:670-3. Available from: http://www.ncbi.nlm.nih.gov/pubmed/24072822%5Cnhttp://www.ncbi.nlm.nih.gov/pubmed/16888140
55. Tackenberg B, Jelcic I, Baerenwaldt A, Oertel W H, Sommer N, Nimmerjahn F, Lunemann J D. Impaired inhibitory Fcgamma receptor IIB expression on B cells in chronic inflammatory demyelinating polyneuropathy. Proc Natl Acad Sci USA [Internet] 2009; 106:4788-92. Available from: http://www.ncbi.nlm.nih.gov/pubmed/19261857%5Cnhttp://www.pubmedcentral.nih.gov/ar ticlerender.fcgi?artid=PMC2660764
56. Wang T T, Maamary J, Tan G S, Bournazos S, Davis C W, Krammer F, Schlesinger S J, Palese P, Ahmed R, Ravetch J V. Anti-HA Glycoforms Drive B Cell Affinity Selection and Determine Influenza Vaccine Efficacy. Cell 2015; 162:160-9.
57. Niezold T, Storcksdieck genannt Bonsmann M, Maaske A, Temchura V, Heinecke V, Hannaman D, Buer J, Ehrhardt C, Hansen W, Überla K, et al. DNA vaccines encoding DEC205-targeted antigens: Immunity or tolerance? Immunology 2015; 145:519-33.
58. Jiang W, Swiggard W J, Heufler C, Peng M, Mirza A, Steinman R M, Nussenzweig M C. The receptor DEC-205 expressed by dendritic cells and thymic epithelial cells is involved in antigen processing. Nature1995; 375:151-5.
59. Coconi-Linares N, Ortega-Dávila E, López-Gonzilez M, García-Machorro J, García-Cordero J, Steinman R M, Cedillo-Barrón L, Gómez-Lim M A. Targeting of envelope domain III protein of DENV type 2 to DEC-205 receptor elicits neutralizing antibodies in mice. Vaccine 2013; 31:2366-71.
60. Nesspor T C, Raju T S, Chin C N, Vafa O, Brezski R J. Avidity confers FcγR binding and immune effector function to aglycosylated immunoglobulin G1. J Mol Recognit 2012; 25:147-54.
61. Wines B D, Vanderven H A, Esparon S E, Kristensen A B, Kent S J, Hogarth P M. Dimeric FcγR Ectodomains as Probes of the Fc Receptor Function of Anti-Influenza Virus IgG. J Immunol [Internet] 2016; 197:1507-16. Available from: http://www.jimmunol.org/lookup/doi/10.4049/jimmunol.1502551
62. Bournazos S, Ravetch J V. Fcγ Receptor Function and the Design of Vaccination Strategies. Immunity2017; 47:224-33.
63. Zhou H, Olsen H, So E, Mdrigeon E, Rybin D, Owens J, LaRosa G, Block D S, Strome S E, Zhang X. A fully recombinant human IgG1 Fc multimer (GL-2045) inhibits complement-mediated cytotoxicity and induces iC3b. Blood Adv [Internet] 2017; 1. Available from: http://www.bloodadvances.org/content/1/8/504?sso-checked-true
64. Sun H, Olsen H S, Mérigeon E Y, So E, Burch E, Kinsey S, Papadimitriou J C, Drachenberg C B, Bentzen S M, Block D S, et al. Recombinant human IgG1 based Fc multimers, with limited FcR binding capacity, can effectively inhibit complement-mediated disease. J Autoimmun 2017; 84:97-108.
65. Yu, X., Vasiljevic, S., Mitchell, D. a., Crispin, M. & Scanlan, C. N. Dissecting the molecular mechanism of IVIg therapy: The interaction between serum IgG and DC-SIGN is independent of antibody glycoform or Fc domain. J. Mol. Biol. 425, 1253-1258 (2013).
66. B. Domon, C. E. Costello, A systematic nomenclature for carbohydrate fragmentations in FAB-MS/MS spectra of glycoconjugates, Glycoconj J, 5 (1988) 397-409.
67. Vanderven, H. A., K. Wragg, F. Ana-Sosa-Batiz, A. B. Kristensen, S. Jegaskanda, A. K. Wheatley, D. Wentworth, B. D. Wines, P. M. Hogarth, S. Rockman, and S. J. Kent. Anti-influenza Hyperimmune Immunoglobulin Enhances Fc-functional Antibody Immunity during Human Influenza Infection. J. Infect. Dis. 1-11. 2018
68. Alborzian Deh Sheikh, A., C. Akatsu, A. Imamura, H. H. M. Abdu-Allah, H. 914 Takematsu, H. Ando, H. Ishida, and T. Tsubata. 2018. Proximity labeling of 915 cis-ligands of CD22/Siglec-2 reveals stepwise α2,6 sialic acid-dependent and 916-independent interactions. Biochem. Biophys. Res. Commun. 495: 854-859.
69. Geijtenbeek, T. B. H., and S. I. Gringhuis. 2009. Signalling through C-type 918 lectin receptors: Shaping immune responses. Nat. Rev. Immunol. 9: 465-479
70. Meyer-Wentrup, F., C. G. Figdor, M. Ansems, P. Brossart, M. D. Wright, G. J. Adema, and A. B. van Spriel. 2007. Dectin-1 Interaction with Tetraspanin C D37 Inhibits IL-6 Production. J. Immunol. 178: 154-162.
71. Karsten, C. M., M. K. Pandey, J. Figge, R. Kilchenstein, P. R. Taylor, M. Rosas, J. U. McDonald, S. J. Orr, M. Berger, D. Petzold, V. Blanchard, A. Winkler, C. Hess, D. M. Reid, I. V Majoul, R. T. Strait, N. L. Harris, G. Köhl, E. Wex, R. Ludwig, D. Zillikens, F. Nimmerjahn, F. D. Finkelman, G. D. Brown, M. Ehlers, and J. Kohl. Anti-inflammatory activity of IgG1 mediated by Fc galactosylation and association of FcγRIIB and dectin-1. Nat. Med. 18:1401-6, 2012.
72. Xu, R., D. C. Ekiert, J. C. Krause, R. Hai, J. E. Crowe, and I. A. Wilson. Structural basis of preexisting immunity to the 2009 H1N1 pandemic influenza virus. Science 328: 357-360, 2010.
73 Hung, I F. N., K. K. W. To, C.-K. Lee, K.-L. Lee, W.-W. Yan, K. Chan, W. M. Chan, C.-W. Ngai, K.-I. Law, F.-L. Chow, R. Liu, K.-Y. Lai, C. C. Y. Lau, S. H. Liu, K.-H. Chan, C.-K. Lin, and K.-Y. Yuen. 2013. Hyperimmune IVimmunoglobulin treatment: a multicenter double-blind randomized controlled trial for patients with severe 2009 influenza A(H1N1) infection. Chest 144:464-473.
74. Luke, T. C., E. M. Kilbane, J. L. Jackson, and S. L. Hoffman. 2006. Meta-analysis: Convalescent blood products for Spanish influenza pneumonia: A future H5N1 treatment. Ann. Intern. Med. 145: 599-609.
75. Vanderven, H. A., K. Wragg, F. Ana-Sosa-Batiz, A. B. Kristensen, S. Jegaskanda, A. K. Wheatley, D. Wentworth, B. D. Wines, P. M. Hogarth, S. Rockman, and S. J. Kent. 2018. Anti-influenza Hyperimmune Immunoglobulin Enhances Fc-functional Antibody Immunity during Human Influenza Infection. J. Infect. Dis. 1-11.
76. Kang, M. C., D.-H. Choi, Y. W. Choi, S. J. Park, H. Namkoong, K. S. Park, S.-S. Ahn, C. D. Surh, S.-W. Yoon, D.-J. Kim, J. Choi, Y. Park, Y. C. Sung, and S.-W. Lee. 2015. Intranasal introduction of Fc-fused interleukin-7 provides long-lasting prophylaxis from lethal influenza infection. J. Virol. 90: 2273-2284. 946
77. Foss, S., A. Grevys, K. M. K. Sand, M. Bern, P. Blundell, T. E. Michaelsen, R. J. Pleass, I. Sandlie, and J. T. Andersen. 2016. Enhanced FcRn-dependent transepithelial delivery of IgG by Fc-engineering and polymerization. J. Control. Release 223: 43-52.
78. Sockolosky, J. T., and F. C. Szoka. 2015. The neonatal Fc receptor, FcRn, as a target for drug delivery and therapy. Adv. Drug Deliv. Rev. 91: 109-124.
79. Patton, J. S., and P. R. Byron. 2007. Inhaling medicines: Delivering drugs to the body through the lungs. Nat. Rev. Drug Discov. 6: 67-74.
80. Heise, T., C. Bull, D. M. Beurskens, E. Rossing, M. I. De Jonge, G. J. Adema, T. J. Boltje, and J. D. Langereis. 2017. Metabolic Oligosaccharide Engineering with Alkyne Sialic Acids Confers Neuraminidase Resistance and Inhibits Influenza Reproduction. Bioconjug. Chem. 28: 1811-1815.
81. Nesspor, T. C., T. S. Raju, C. N. Chin, O. Vafa, and R. J. Brezski. 2012. Avidity confers FcγR binding and immune effector function to aglycosylated immunoglobulin G1. J. Mol. Recognit. 25: 147-154.
82. Wines, B. D., H. A. Vanderven, S. E. Esparon, A. B. Kristensen, S. J. Kent, and P. M. Hogarth. 2016. Dimeric FcγR Ectodomains as Probes of the Fc Receptor Function of Anti-Influenza Virus IgG. J. Immunol. 197: 1507-1516.
83. Bournazos, S., and J. V. Ravetch. 2017. Fcγ Receptor Function and the Design of Vaccination Strategies. Immunity 47: 224-233.
84. North, S. J., J. Jang-Lee, R. Harrison, K. Canis, M. N. Ismail, A. Trollope, A. Antonopoulos, P. C. Pang, P. Grassi, S. Al-Chalabi, A. T. Etienne, A. Dell, and S. M. Haslam. 2010. Mass spectrometric analysis of mutant mice. Methods Enzymol. 478: 27-77.
84. Ceroni, A., K. Maass, H. Geyer, R. Geyer, A. Dell, and S. M. Haslam. 2008. GlycoWorkbench: A tool for the computer-assisted annotation of mass spectra of glycans. J. Proteome Res. 7: 1650-1659
85. Khatua et al. Indian J. Medical Res. 138:648-662, 2013.
86. Roy S, Mandal C. PLoS Negl Trop Dis. 10(8):e0004904. doi: 10.1371/journal.pntd.0004904. eCollection 2016 August

### EMBODIMENTS

1. A chimeric Fc receptor binding protein comprising two chimeric polypeptide chains; wherein each chimeric polypeptide chain comprises a hinge region, an immunoglobulin G heavy chain constant region and an immunoglobulin tailpiece region, wherein the amino acid sequence of each polypeptide chain is altered as compared to the native sequences from which the immunoglobulin G heavy chain constant region and the immunoglobulin tailpiece region are derived to remove cysteine residues that form extrinsic disulphide bonds, and wherein each polypeptide chain comprises at least one glycosylation site.

2. A protein according to claim 1, wherein the alteration includes the loss of a cysteine residue corresponding to residue 248 of SEQ ID NO:1.

3. A protein according to any of claim 1 or 2, wherein the cysteine residue corresponding to residue 248 of SEQ ID NO: 1, is replaced with an alanine residue.

4. A protein according to any of the preceding claims, wherein each polypeptide chain is altered to introduce a glycosylation site in one of the regions that is not present in the native sequence from which the region is derived.

5. A protein according to any of the preceding claims, wherein each polypeptide chain comprises a glycosylation site in the hinge region that is not present in the native sequence from which the hinge region is derived.

6. A protein according to claim 5, wherein the additional glycosylation site is at a position that corresponds to residue 1 of SEQ ID NO:1.

7. A protein according to any one of claims 4-6, wherein the glycosylation site is an asparagine residue.

8. A protein according to any of the preceding claims, wherein the amino acid sequence of each polypeptide is altered as compared to the native sequences from which the hinge region, immunoglobulin G heavy chain constant region and the immunoglobulin tailpiece region are derived to possess one fewer glycosylation site.

9. A protein according to claim 8, wherein as compared to the native immunoglobulin sequence there is a loss of the asparagine residue corresponding to residue 77 of SEQ ID NO:1.

10. A protein according to claim 8, wherein as compared to the native immunoglobulin sequence there is a loss of the asparagine residue corresponding to residue 236 of SEQ ID NO:1.

11. A protein according to any of the preceding claims, wherein the amino acid sequence of each polypeptide is altered as compared to the native sequences from which the hinge region, immunoglobulin G heavy chain constant region and the immunoglobulin tailpiece region are derived to possess two fewer glycosylation sites.

12. A protein according to claim 11, wherein as compared to the native immunoglobulin sequences there is a loss of the asparagine residue corresponding to residue 77 of SEQ ID NO:1 and residue 236 of SEQ ID NO:1.

13. A protein according to any of the preceding claims, wherein there is a glycosylation site at a position that corresponds to residue 77 of SEQ ID NO: 1.

14. A protein according to any of the preceding claims, wherein there is a glycosylation site at a position that corresponds to residue 236 of SEQ ID NO: 1.

15. A protein according to any of the preceding claims, which comprises three glycosylation sites located at position 1, 77 and 236 corresponding to the position in SEQ ID NO: 1.

16. A protein according to any of the preceding claims, wherein each polypeptide chain comprises two immunoglobulin G heavy chain constant domains.

17. A protein according to any of the preceding claims, wherein the protein is in monomeric form.

18. A protein according to any preceding claim, wherein the tailpiece is based upon the tailpiece of an immunoglobulin selected from the group consisting of: IgM, IgA, and IgE.

19. A protein according to claim 18, wherein the tailpiece is based upon the tailpiece of IgM.

20. A protein according to any preceding claim, wherein the tailpiece shares at least 70% identity with a native immunoglobulin tailpiece.

21. A protein according to any preceding claim, wherein the tailpiece shares at least 70% identity with amino acid residues 232-249 of SEQ ID NO:2.

22. A protein according to claim 21, wherein the tailpiece shares at least 90% identity with amino acid residues 232-249 of SEQ ID NO:2.

23. A protein according to any preceding claim, wherein the immunoglobulin G heavy chain constant region is derived from an immunoglobulin selected from the group consisting of: IgG1, IgG2, IgG3 and IgG4.

24. A protein according to claim 23, wherein the immunoglobulin G heavy chain constant regions are derived from IgG1.

25. A protein according to any preceding claim, wherein the IgG derived sequences share at least 70% sequence identity with the native IgG sequence from which they are derived.

26. A protein according to any preceding claim comprising a sequence selected from: SEQ ID NO:2-8.

27. A protein according to any preceding claim consisting of a sequence selected from: SEQ ID NO:2-8.

28. A chimeric Fc receptor binding protein comprising two chimeric polypeptide chains; wherein each chimeric polypeptide chain comprises a hinge region, an immunoglobulin G heavy chain constant region and an immunoglobulin tailpiece region, wherein the amino acid sequence of each polypeptide chain is altered as compared to the native sequences from which the hinge region, immunoglobulin G heavy chain constant region and the immunoglobulin tailpiece region are derived to possess an additional glycosylation site in the hinge region corresponding to residue 1 of SEQ ID NO: 1, and the loss of the cysteine residue corresponding to residue 248 of SEQ ID NO: 1.

29. A protein according to any preceding claim, wherein at least one of the polypeptide chains is conjugated to a therapeutic payload.

30. A protein according to claim 29, wherein the therapeutic payload is conjugated to the hinge region.

31. A protein according to claim 29 or 30, wherein the therapeutic payload is an immune modulator.

32. A protein according to claim 29 or 30, wherein the therapeutic payload comprises an antigen-binding domain.

33. A protein according to claim 29 or 30, wherein the antigen-binding domain is selected from the group consisting of Fab, F(ab′)2, Fd, Fv, scFV, dAb and nanobody.

34. A protein in accordance with any of claims 1 to 33, for use as a medicament.

35. A protein for use in accordance with claim 34, for use in intravenous immunoglobulin (IVIG) or subcutaneous immunoglobulin (SCIG) therapy.

36. A protein for use in accordance with claim 34 or claim 35, for use in the prevention and/or treatment of autoimmune or inflammatory diseases.

37. A protein for use in accordance with any of claims 34 to 36, for use in the prevention and/or treatment of autoimmune or inflammatory diseases selected from the group consisting of autoimmune cytopenias, Guillain-Barré syndrome, myasthenia gravis, anti-Factor VIII autoimmune disease, dermatomyositis, vasculitis, and uveitis.

38. A protein for use according to claim 34, for use as a vaccine.

39. A protein for use according to claim 34, for use in the prevention or treatment of a disease mediated through binding of sialic acid-dependent receptors.

40. A protein for use according to claim 34, for use in the prevention or treatment of infection.

41. A protein for use according to claim 34, for use in the prevention or treatment of a retroviral infection.

42. A composition comprising a protein according to any of claims 1 to 33, wherein at least 95% of the protein incorporated in the composition is in monomeric form.

43. A method of preventing or treating an autoimmune or inflammatory disease, the method comprising providing a therapeutically effective amount of protein in accordance with any of claims 1 to 33 to a subject in need of such prevention or treatment.

44. A method according to claim 43, wherein the subject is a human subject.

45. A method according to claim 43 or claim 44, wherein the proteins is provided in intravenous immunoglobulin (IVIG) or subcutaneous immunoglobulin (SCIG) therapy.

46. A method of preventing or treating a disease, the method comprising providing a therapeutically effective amount of a protein in accordance with any of claims 1 to 33 as a vaccine to a subject in need of such treatment.

47. A method according to claim 46, wherein the protein is conjugated to an immune modulator.

48. A method of preventing or treating a disease mediated through binding of sialic acid-dependent receptors, the method comprising providing a therapeutically effective amount of a protein in accordance with any of claims 1 to 33 as a vaccine to a subject in need of such treatment.

49. A method according to claim 48, wherein the disease is an infection.

50. A method according to claim 49, wherein the infection is a retroviral infection.

51. A nucleic acid encoding a protein according to any of claims 1 to 33.

52. A nucleic acid according to claim 51, for use as a medicament.

53. A method of producing a protein according to any of claims 1 to 33, the method comprising expressing a nucleic acid in accordance with claim 51 in a host cell.

54. A chimeric Fc receptor binding protein comprising two chimeric polypeptide chains; wherein each chimeric polypeptide chain comprises a hinge region, an immunoglobulin G heavy chain constant region and an immunoglobulin tailpiece, wherein the amino acid sequence of each polypeptide chain is altered as compared to the native sequences from which the immunoglobulin G heavy chain constant region and the immunoglobulin tailpiece region are derived to possess at least two fewer glycosylation sites.

55. A protein according to claim 54, wherein each polypeptide chains also comprises a glycosylation site at position 1 according to SEQ ID NO: 1 and lacks a cysteein the tailpiece capable of forming an extrinsic disulphide bond.

56. A protein according to claim 54 or 55, wherein as compared to the native immunoglobulin sequences from which the immunoglobulin G heavy chain constant region and the immunoglobulin tailpiece region are derived there is a loss of the asparagine residue at the position corresponding to residue 77 of SEQ ID NO:1 and residue 236 of SEQ ID NO:1.

57. A protein according to any of claims 54 to 56, wherein the protein is capable of binding FcγRI and FcγRIIB.

58. A protein according to any of claims 54 to 57, wherein the protein is capable of binding C1q.

1. A chimeric Fc receptor binding protein or a pharmaceutical composition comprising said chimeric Fc receptor binding protein for use in the treatment or prevention of a disease mediated by a pathogen that relies on sialic acid receptors interactions, wherein the chimeric Fc receptor binding protein comprises two chimeric polypeptide chains, wherein each chimeric polypeptide chain comprises an immunoglobulin G heavy chain constant region, a tailpiece region and a hinge region, wherein the amino acid sequence of each polypeptide chain possess a sugar moiety at or close to the N-terminus and a sugar moiety at or close to the C-terminus.

2. The chimeric Fc receptor binding protein or pharmaceutical composition comprising said chimeric Fc receptor binding protein for use according to any of claim 1, wherein the sugar moieties are each attached to a separate amino acid of the polypeptide.

3. The chimeric Fc receptor binding protein or pharmaceutical composition comprising said chimeric Fc receptor binding protein for use according to any of claim 1 or 2, wherein the sugar moieties are separated by 60 Å±5 Å.

4. The chimeric Fc receptor binding protein or pharmaceutical composition comprising said chimeric Fc receptor binding protein for use according to any of claims 1, 2 or 3, wherein the sugar moiety attached at or close to the N-terminus is attached to one of the amino acids at a position from 1 to 12 from and including the N-terminus.

5. The chimeric Fc receptor binding protein or pharmaceutical composition comprising said chimeric Fc receptor binding protein for use according to any of claims 1, 2, 3 or 4, wherein the sugar moiety attached at or close to the C-terminus is attached to the amino acid at a position from −12 to −3 from and including the C-terminus.

6. The chimeric Fc receptor binding protein or pharmaceutical composition comprising said chimeric Fc receptor binding protein for use according to any of the preceding claims, wherein the sugar moieties are each attached to an asparagine amino acid.

7. The chimeric Fc receptor binding protein or pharmaceutical composition comprising said chimeric Fc receptor binding protein for use according to claim 6, wherein the asparagine residue is at position 1 according to the sequence disclosed in SEQ ID NO: 1.

8. The chimeric Fc receptor binding protein or pharmaceutical composition comprising said chimeric Fc receptor binding protein for use according to any of claim 6, wherein the asparagine residue is at position 236 according to the sequence disclosed in SEQ ID NO: 1.

9. The chimeric Fc receptor binding protein or pharmaceutical composition comprising said chimeric Fc receptor binding protein for use according to any of the preceding claims, wherein one or both chimeric polypeptide chains comprises or consists of a sequence as disclosed in SEQ ID NO: 9 or 59.

10. The chimeric Fc receptor binding protein or pharmaceutical composition comprising said chimeric Fc receptor binding protein for use according to any of the preceding claims, wherein the immunoglobulin G heavy chain constant region and tailpiece region lack cysteine amino acids.

11. The chimeric Fc receptor binding protein or pharmaceutical composition comprising said chimeric Fc receptor binding protein for use according to any of the preceding claims, wherein the chimeric Fc receptor binding protein is selected from the molecule referred to as: D221N/N297A/N563A/C575A or D221N/C575A.

12. The chimeric Fc receptor binding protein or pharmaceutical composition comprising said chimeric Fc receptor binding protein for use according to any of the preceding claims, wherein the pathogen is selected from a bacterium, parasite or virus.

13. The chimeric Fc receptor binding protein or pharmaceutical composition comprising said chimeric Fc receptor binding protein for use according to claim 12, wherein the bacterium is selected from: Streptococcus pneumoniae, Streptococcus suis, Streptococcus agalactiae, Neisseria meningitidis, E. coli, Campylobacter jejuni, Pseudomonas aeruginosa, Haemophilus influenzae, Haemophilus ducrey, Helicobacter pylori, Legionella pneumophila, Pasteurella multocida, Salmonella enterica, Vibrio cholerae and Neospora caninum.

14. The chimeric Fc receptor binding protein or pharmaceutical composition comprising said chimeric Fc receptor binding protein for use according to claim 12, wherein the parasite is Plasmodium falciparum, Trypanosoma cruzi, Toxoplasma gondii and Leishmania donovani.

15. The chimeric Fc receptor binding protein or pharmaceutical composition comprising said chimeric Fc receptor binding protein for use according to claim 12, wherein the virus is selected from influenza, Porcine Reproductive and Respiratory Syndrome Virus, rotavirus, Paramyxovirus (such as Newcastle disease virus and rubulavirus), norovirus, enterovirus, rotavirus, polyomavirus (such as e.g. Merkel cell polyomavirus), coronaviruses (including Mers and Sars), adenovirus (such as Ad37) and lentivirus (such as HIV-1).

16. The chimeric Fc receptor binding protein or pharmaceutical composition comprising said chimeric Fc receptor binding protein for use according to any of the preceding claims, wherein the tailpiece region is an immunoglobulin tailpiece region.

17. The chimeric Fc receptor binding protein or pharmaceutical composition comprising said chimeric Fc receptor binding protein for use according to claim 16, wherein the immunoglobulin tailpiece region is from IgM.

18. The chimeric Fc receptor binding protein or pharmaceutical composition comprising said chimeric Fc receptor binding protein for use according to any of the preceding claims, wherein the tailpiece region is a polypeptide sequence of from 12 to 26 amino acids in length.

19. A chimeric Fc receptor binding protein comprising two chimeric polypeptide chains, wherein each chimeric polypeptide chain comprises an immunoglobulin G heavy chain constant region, a tailpiece region and a hinge region, wherein the amino acid sequence of each polypeptide chain possess a sugar moiety at or close to the N-terminus and a sugar moiety at or close to the C-terminus.

20. The chimeric Fc receptor binding protein according to claim 19, wherein the sugar moieties are each attached to a separate amino acid of the polypeptide.

21. The chimeric Fc receptor binding protein according to claim 19 or 20, wherein the sugar moieties are separated by 60 Å 5 Å.

22. The chimeric Fc receptor binding protein according to any of claims 19, 20 or 21, wherein the sugar moiety attached at or close to the N-terminus is attached to one of the amino acids at a position from 1 to 12 from and including the N-terminus.

23. The chimeric Fc receptor binding protein according to claim 22, wherein the sugar moiety attached at or close to the C-terminus is attached to the amino acid at a position from −12 to −3 from and including the C-terminus.

24. The chimeric Fc receptor binding protein according to any of claims 19 to 23, wherein the sugar moieties are each attached to an asparagine amino acid.

25. The chimeric Fc receptor binding protein according to claim 24, wherein the asparagine residue is at position 1 according to the sequence disclosed in SEQ ID NO: 1.

26. The chimeric Fc receptor binding protein according to claim 24, wherein the asparagine residue is at position 236 according to the sequence disclosed in SEQ ID NO: 1.

27. The chimeric Fc receptor binding protein according to any of claims 19 to 26, wherein one or both chimeric polypeptide chains comprises or consists of a sequence as disclosed in SEQ ID NO: 9 or 59.

28. The chimeric Fc receptor binding protein according to any of claims 19 to 27, wherein the immunoglobulin G heavy chain constant region and tailpiece region lack cysteine amino acids.

29. The chimeric Fc receptor binding protein according to any of claims 19 to 28, wherein the chimeric Fc receptor binding protein is selected from the molecule referred to as: D221N/N297A/N563A/C575A or D221N/C575A.

30. The chimeric Fc receptor binding protein according to any of claims 19 to 29, wherein the tailpiece region is an immunoglobulin tailpiece region.

31. The chimeric Fc receptor binding protein according to claim 30, wherein the immunoglobulin tailpiece region is an IgM tailpiece.

32. The chimeric Fc receptor binding protein according to any of claims 19 to 31, wherein the tailpiece region is a polypeptide sequence of from 12 to 26 amino acids in length.

33. A chimeric Fc receptor binding protein comprising two chimeric polypeptide chains; wherein each chimeric polypeptide chain comprises a hinge region, an immunoglobulin G heavy chain constant region and an immunoglobulin tailpiece region, wherein the amino acid sequence of each polypeptide chain is altered as compared to the native sequences from which the immunoglobulin G heavy chain constant region and the immunoglobulin tailpiece region are derived to remove cysteine residues that form extrinsic disulphide bonds, and wherein each polypeptide chain comprises at least one glycosylation site.

34. The chimeric Fc receptor binding protein according to claim 33, wherein the alteration includes the loss of a cysteine residue corresponding to residue 248 of SEQ ID NO:1, optionally wherein the cysteine residue corresponding to residue 248 of SEQ ID NO: 1, is replaced with an alanine residue

35. The chimeric Fc receptor binding protein according to claim 33 or 34, wherein each polypeptide chain is altered to introduce a glycosylation site in one of the regions that is not present in the native sequence from which the region is derived.

36. The chimeric Fc receptor binding protein according to claims 33, 34 or 35, wherein each polypeptide chain comprises a glycosylation site in the hinge region that is not present in the native sequence from which the hinge region is derived.

37. The chimeric Fc receptor binding protein according to claim 36, wherein the additional glycosylation site is at a position that corresponds to residue 1 of SEQ ID NO:1.

38. The chimeric Fc receptor binding protein according to any of claims 33 to 35, wherein the amino acid sequence of each polypeptide is altered as compared to the native sequences from which the hinge region, immunoglobulin G heavy chain constant region and the immunoglobulin tailpiece region are derived to possess one or two fewer glycosylation site.

39. The chimeric Fc receptor binding protein according to claim 6, wherein as compared to the native immunoglobulin sequence there is (i) a loss of the asparagine residue corresponding to residue 77 of SEQ ID NO:1; and/or (ii) a loss of the asparagine residue corresponding to residue 236 of SEQ ID NO:1.

40. The chimeric Fc receptor binding protein according to any preceding claim comprising or consisting of a sequence selected from: SEQ ID NO: 2-8.

41. A chimeric Fc receptor binding protein comprising two chimeric polypeptide chains; wherein each chimeric polypeptide chain comprises a hinge region, an immunoglobulin G heavy chain constant region and an immunoglobulin tailpiece region, wherein the amino acid sequence of each polypeptide chain is altered as compared to the native sequences from which the hinge region, immunoglobulin G heavy chain constant region and the immunoglobulin tailpiece region are derived to possess an additional glycosylation site in the hinge region corresponding to residue 1 of SEQ ID NO: 1, and the loss of the cysteine residue corresponding to residue 248 of SEQ ID NO: 1.

42. A chimeric Fc receptor binding protein comprising two chimeric polypeptide chains; wherein each chimeric polypeptide chain comprises a hinge region, an immunoglobulin G heavy chain constant region and an immunoglobulin tailpiece, wherein the amino acid sequence of each polypeptide chain is altered as compared to the native sequences from which the immunoglobulin G heavy chain constant region and the immunoglobulin tailpiece region are derived to possess at least two fewer glycosylation sites, and optionally to also lack cysteine residues in the tailpiece region.

43. The chimeric Fc receptor binding protein according to claim 42, wherein each polypeptide chains also comprises a glycosylation site at position 1 according to SEQ ID NO: 1 and lacks a cysteiein the tailpiece capable of forming an extrinsic disulphide bond.

44. The chimeric Fc receptor binding protein according to claim 42 or 43, wherein as compared to the native immunoglobulin sequences from which the immunoglobulin G heavy chain constant region and the immunoglobulin tailpiece region are derived there is a loss of the asparagine residue at the position corresponding to residue 77 of SEQ ID NO:1 and residue 236 of SEQ ID NO:1.

45. The chimeric Fc receptor binding protein according to any of claims 42 to 44, wherein the protein is capable of binding FcγRI and/or FcγRIIIA and/or FcγRIIB and/or C1q.

46. The chimeric Fc receptor binding protein according to any of claims 19 to 45, wherein at least one of the polypeptide chains is conjugated to a therapeutic payload.

47. The chimeric Fc receptor binding protein according to claim 46, wherein the therapeutic payload is conjugated to the hinge region.

48. A nucleic acid encoding a protein according to any of claims 19 to 47.

49. A method of producing a protein according to any of claims 19 to 47, the method comprising expressing a nucleic acid in accordance with claim 48 in a host cell.

50. The method according to claim 49, wherein the host cell is a CHO-K1 cell.

51. A pharmaceutical composition comprising a protein according to any of claims 19 to 47, and at least one pharmaceutically acceptable carrier.

52. The composition according to claim 51, wherein at least 95% of the protein incorporated in the composition is in monomeric form.

53. The chimeric Fc receptor binding protein in accordance with any of claims 19 to 47, or the pharmaceutical composition according to claim 48 for use as a medicament.

54. The chimeric Fc receptor binding protein or a pharmaceutical composition thereof for use in accordance with claim 53, for use in intravenous immunoglobulin (IVIG) or subcutaneous immunoglobulin (SCIG) therapy.

55. The chimeric Fc receptor binding protein or a pharmaceutical composition thereof for use in accordance with claim 53, for use in the prevention and/or treatment of autoimmune or inflammatory diseases, such as one selected from the group consisting of: autoimmune cytopenias, Guillain-Barré syndrome, myasthenia gravis, anti-Factor VIII autoimmune disease, dermatomyositis, vasculitis, and uveitis.

56. The chimeric Fc receptor binding protein or a pharmaceutical composition thereof for use according to claim 53, for use as a vaccine.

57. The chimeric Fc receptor binding protein or a pharmaceutical composition thereof for use according to claim 53, for use in the prevention or treatment of a disease mediated by a pathogen through binding of sialic acid-dependent receptors.

58. The chimeric Fc receptor binding protein or a pharmaceutical composition thereof for use according to claim 57, wherein the pathogen is selected from a bacterium, parasite or virus.

59. The chimeric Fc receptor binding protein or a pharmaceutical composition thereof for use according to claim 58, wherein the bacterium is selected from the group consisting of: Streptococcus pneumoniae, Streptococcus suis, Streptococcus agalactiae, Neisseria meningitidis, E. coli, Campylobacter jejuni, Pseudomonas aeruginosa, Haemophilus influenzae, Haemophilus ducrey, Helicobacter pylori, Legionella pneumophila, Pasteurella multocida, Salmonella enterica, Vibrio cholerae and Neospora caninum.

60. The chimeric Fc receptor binding protein or a pharmaceutical composition thereof for use according to claim 58, wherein the parasite is selected from the group consisting of: Plasmodium falciparum, Trypanosoma cruzi, Toxoplasma gondii and Leishmania donovani.

61. The chimeric Fc receptor binding protein or a pharmaceutical composition thereof for use according to claim 58, wherein the virus is selected from the group consisting of: influenza, Porcine Reproductive and Respiratory Syndrome Virus, rotavirus, Paramyxovirus (such as Newcastle disease virus and rubulavirus), norovirus, enterovirus, rotavirus, polyomavirus (such as e.g. Merkel cell polyomavirus), coronaviruses (including Mers and Sars), adenovirus (such as Ad37) and lentivirus (such as HIV-1).

62. The chimeric Fc receptor binding protein or a pharmaceutical composition thereof for use according to claim 53, wherein the use is in the prevention or treatment of infection.

63. The chimeric Fc receptor binding protein or a pharmaceutical composition thereof for use according to claim 62, for use in the prevention or treatment of a retroviral infection.

64. A method of preventing or treating an autoimmune or inflammatory disease, the method comprising providing a therapeutically effective amount of a chimeric Fc receptor binding protein in accordance with any of claims 19 to 47 or a pharmaceutical composition in accordance with claim 48, to a subject in need of such prevention or treatment.

65. The method according to claim 64, wherein the chimeric Fc receptor binding protein is provided in intravenous immunoglobulin (IVIG) or subcutaneous immunoglobulin (SCIG) therapy.

66. A method of preventing or treating a disease, the method comprising providing a therapeutically effective amount of the chimeric Fc receptor binding protein in accordance with any of claims 19 to 47 or a pharmaceutical composition in accordance with claim 48, as a vaccine to a subject in need of such treatment.

67. The method according to claim 66, wherein the chimeric Fc receptor binding protein is conjugated to an immune modulator.

68. The method of preventing or treating a disease mediated by a pathogen through binding of sialic acid-dependent receptors, the method comprising providing a therapeutically effective amount of a protein in accordance with any of claims 19 to 47 or a pharmaceutical composition in accordance with claim 48, as a vaccine to a subject in need of such treatment.

69. The method according to claim 68, wherein the disease is an infection, such as a retroviral infection.


SEQUENCE LISTING (60 sequences)

SEQ ID NO: 1 - protein, 249 aa, Artificial Sequence
  Reference protein Hexa-Fc or HexaGard
DKTHTCPPCPAPELLGGPSVFLFPPKPKDTLMISRTPEVTCVVVDVSHEDPEVKFNWYVDGVEVHNAKTKPREEQYNSTYRVVSVLTVCLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTCY

SEQ ID NO: 2 - protein, 249 aa, Artificial Sequence
  C309L/C575A
DKTHTCPPCPAPELLGGPSVFLFPPKPKDTLMISRTPEVTCVVVDVSHEDPEVKFNWYVDGVEVHNAKTKPREEQYNSTYRVVSVLTVLLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTAY

SEQ ID NO: 3 - protein, 249 aa, Artificial Sequence
  D221N/C309L/C575A
NKTHTCPPCPAPELLGGPSVFLFPPKPKDTLMISRTPEVTCVVVDVSHEDPEVKFNWYVDGVEVHNAKTKPREEQYNSTYRVVSVLTVLLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTAY

SEQ ID NO: 4 - protein, 249 aa, Artificial Sequence
  C309L/N563A/C575A
DKTHTCPPCPAPELLGGPSVFLFPPKPKDTLMISRTPEVTCVVVDVSHEDPEVKFNWYVDGVEVHNAKTKPREEQYNSTYRVVSVLTVLLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYAVSLVMSDTAGTAY

SEQ ID NO: 5 - protein, 249 aa, Artificial Sequence
  D221N/C309L/N297A/N563A/C575A
NKTHTCPPCPAPELLGGPSVFLFPPKPKDTLMISRTPEVTCVVVDVSHEDPEVKFNWYVDGVEVHNAKTKPREEQYASTYRVVSVLTVLLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYAVSLVMSDTAGTAY

SEQ ID NO: 6 - protein, 249 aa, Artificial Sequence
  D221N/C309L/N563A/C575A
NKTHTCPPCPAPELLGGPSVFLFPPKPKDTLMISRTPEVTCVVVDVSHEDPEVKFNWYVDGVEVHNAKTKPREEQYNSTYRVVSVLTVLLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYAVSLVMSDTAGTAY

SEQ ID NO: 7 - protein, 249 aa, Artificial Sequence
  - D221N/C309L/N297A/C575A
NKTHTCPPCPAPELLGGPSVFLFPPKPKDTLMISRTPEVTCVVVDVSHEDPEVKFNWYVDGVEVHNAKTKPREEQYASTYRVVSVLTVLLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTAY

SEQ ID NO: 8 - protein, 249 aa, Artificial Sequence
  C309L/N297A/C575A
DKTHTCPPCPAPELLGGPSVFLFPPKPKDTLMISRTPEVTCVVVDVSHEDPEVKFNWYVDGVEVHNAKTKPREEQYASTYRVVSVLTVLLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTAY

SEQ ID NO: 9 - protein, 249 aa, Artificial Sequence
  D221N/N297A/N563A/C575A
NKTHTCPPCPAPELLGGPSVFLFPPKPKDTLMISRTPEVTCVVVDVSHEDPEVKFNWYVDGVEVHNAKTKPREEQYASTYRVVSVLTVCLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYAVSLVMSDTAGTAY

SEQ ID NO: 10 - protein, 249 aa, Artificial Sequence
  N297A/N563A/C575A
DKTHTCPPCPAPELLGGPSVFLFPPKPKDTLMISRTPEVTCVVVDVSHEDPEVKFNWYVDGVEVHNAKTKPREEQYASTYRVVSVLTVCLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYAVSLVMSDTAGTAY

SEQ ID NO: 11 - protein, 249 aa, Artificial Sequence
  C309L
DKTHTCPPCPAPELLGGPSVFLFPPKPKDTLMISRTPEVTCVVVDVSHEDPEVKFNWYVDGVEVHNAKTKPREEQYNSTYRVVSVLTVLLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTCY

SEQ ID NO: 12 - DNA, 750 bp, Artificial Sequence
  nucleic acid encoding seq 1 (Hexa-Fc)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtctgcctccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacaacgtgtccctggtcatgtccgacacagctggcacctgctactga

SEQ ID NO: 13 - DNA, 750 bp, Artificial Sequence
  nucleic acid encoding C309L/C575A
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcttgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacaacgtgtccctggtcatgtccgacacagctggcacctgctactga

SEQ ID NO: 14 - protein, 18 aa, Homo sapiens
PTLYNVSLVMSDTAGTCY

SEQ ID NO: 15 - protein, 18 aa, Homo sapiens
PTHVNVSVVMAQVDGTCY

SEQ ID NO: 16 - protein, 4 aa, Artificial Sequence
  spacer region
LVLG

SEQ ID NO: 17 - protein, 23 aa, Homo sapiens
EPKSCDKTHTCPPCPAPELLGGP

SEQ ID NO: 18 - protein, 10 aa, Homo sapiens
DKTHTCPPCP

SEQ ID NO: 19 - protein, 19 aa, Artificial Sequence
  hinge region
VPSTPPTPSPSTPPTPSPS

SEQ ID NO: 20 - protein, 6 aa, Artificial Sequence
  hinge region
VPPPPP

SEQ ID NO: 21 - protein, 15 aa, Artificial Sequence
  hinge region
EPKSCDKTHTCPPCP

SEQ ID NO: 22 - protein, 12 aa, Artificial Sequence
  hinge region
ERKCCVECPPCP

SEQ ID NO: 23 - protein, 12 aa, Artificial Sequence
  hinge region
ESKYGPPCPSCP

SEQ ID NO: 24 - protein, 4 aa, Artificial Sequence
  hinge region
CPPC

SEQ ID NO: 25 - protein, 4 aa, Artificial Sequence
  hinge region
CPSC

SEQ ID NO: 26 - protein, 4 aa, Artificial Sequence
  hinge region
SPPC

SEQ ID NO: 27 - protein, 10 aa, Artificial Sequence
  hinge region
DKTHTCPPCP

SEQ ID NO: 28 - protein, 18 aa, Artificial Sequence
  exemplary protein of the invention
DKTHTCPPCPAPELLGGP

SEQ ID NO: 29 - DNA, 19 bp, Artificial Sequence
  Forward Primer
accctgcttgctcaactct

SEQ ID NO: 30 - DNA, 29 bp, Artificial Sequence
  Reverse Primer
ggccagctagctcagtaggcggtgccagc

SEQ ID NO: 31 - DNA, 21 bp, Artificial Sequence
  Forward Primer
tcaccgtcttgcaccaggact

SEQ ID NO: 32 - DNA, 21 bp, Artificial Sequence
  Reverse Primer
agtcctggtgcaagacggtga

SEQ ID NO: 33 - DNA, 21 bp, Artificial Sequence
  N297A Forward Primer
gagcagtacgccagcacgtac

SEQ ID NO: 34 - DNA, 21 bp, Artificial Sequence
  N297A Reverse Primer
ctcgtcatgcggtcgtgcatg

SEQ ID NO: 35 - DNA, 20 bp, Artificial Sequence
  N563A Forward Primer
ccctgtacgccgtgtccctg

SEQ ID NO: 36 - DNA, 20 bp, Artificial Sequence
  N563A Reverse Primer
gggacatgcggcacagggac

SEQ ID NO: 37 - DNA, 21 bp, Artificial Sequence
  D221N Forward Primer
gttagatctaacaaaactcac

SEQ ID NO: 38 - DNA, 21 bp, Artificial Sequence
  D221N Reverse Primer
caatctagattgttttgagtg

SEQ ID NO: 39 - DNA, 19 bp, Artificial Sequence
  C575A Forward Primer
accctgcttgctcaactct

SEQ ID NO: 40 - DNA, 29 bp, Artificial Sequence
  C575A reverse primer
ggccagctagctcagtaggcggtgccagc

SEQ ID NO: 41 - DNA, 21 bp, Artificial Sequence
  C309L Forward Primer
tcaccgtcttgcaccaggact

SEQ ID NO: 42 - DNA, 21 bp, Artificial Sequence
  C309L Reverse Primer
agtcctggtgcaagacggtga

SEQ ID NO: 43 - DNA, 19 bp, Artificial Sequence
  Overlap PCR Forward Primer
accctgcttgctcaactct

SEQ ID NO: 44 - DNA, 21 bp, Artificial Sequence
  overlap PCR Reverse Primer
tggtttgtccaaactcatcaa

SEQ ID NO: 45 - protein, 18 aa, Artificial Sequence
  modified  “tailpiece”
PTHVNVSVVMAEVDGTCY

SEQ ID NO: 46 - DNA, 750 bp, Artificial Sequence
  nucleic acid encoding D221N/C309L/C575A
aacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcttgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacaacgtgtccctggtcatgtccgacacagctggcaccgcctactga

SEQ ID NO: 47 - DNA, 750 bp, Artificial Sequence
  nucleic acid encoding C309L/N563A/C575A
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcttgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacgccgtgtccctggtcatgtccgacacagctggcaccgcctactga

SEQ ID NO: 48 - DNA, 750 bp, Artificial Sequence
  nucleic acid encoding D221N/C309L/N297A/N563A/ C575A
aacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcttgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacgccgtgtccctggtcatgtccgacacagctggcaccgcctactga

SEQ ID NO: 49 - DNA, 750 bp, Artificial Sequence
  nucleic acid encoding D221N/C309L/N563A/C575A
aacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcttgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacgccgtgtccctggtcatgtccgacacagctggcaccgcctactga

SEQ ID NO: 50 - DNA, 750 bp, Artificial Sequence
  nucleic acid encoding D221N/C309L/N297A/C575A
aacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcttgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacaacgtgtccctggtcatgtccgacacagctggcaccgcctactga

SEQ ID NO: 51 - DNA, 750 bp, Artificial Sequence
  nucleic acid encoding C309L/N297A/C575A
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcttgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacaacgtgtccctggtcatgtccgacacagctggcaccgcctactga

SEQ ID NO: 52 - DNA, 750 bp, Artificial Sequence
  nucleic acid encoding D221N/N297A/N563A/C575A
aacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtctgcctccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacgccgtgtccctggtcatgtccgacacagctggcaccgcctactga

SEQ ID NO: 53 - DNA, 750 bp, Artificial Sequence
  nucleic acid encoding N297A/N563A/C575A
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtctgcctccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacgccgtgtccctggtcatgtccgacacagctggcaccgcctactga

SEQ ID NO: 54 - DNA, 750 bp, Artificial Sequence; identical to SEQ ID NO: 13
  nucleic acid encoding C309L

SEQ ID NO: 55 - protein, 249 aa, Artificial Sequence
  C309L/N297A/N563A/C575A
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYAVSLVMSDTAGTAY

SEQ ID NO: 56 - DNA, 750 bp, Artificial Sequence
  nucleic acid encoding C309L/N297A/N563A/C575A
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcttgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacgccgtgtccctggtcatgtccgacacagctggcaccgcctactga

SEQ ID NO: 57 - protein, 249 aa, Artificial Sequence
  N297A/C575A
DKTHTCPPCPAPELLGGPSVFLFPPKPKDTLMISRTPEVTCVVVDVSHEDPEVKFNWYVDGVEVHNAKTKPREEQYASTYRVVSVLTVCLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTAY

SEQ ID NO: 58 - DNA, 750 bp, Artificial Sequence
  nucleic acid encoding N297A/C575A
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtctgcctccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacaacgtgtccctggtcatgtccgacacagctggcaccgcctactga

SEQ ID NO: 59 - protein, 249 aa, Artificial Sequence
  D221N/C575A
NKTHTCPPCPAPELLGGPSVFLFPPKPKDTLMISRTPEVTCVVVDVSHEDPEVKFNWYVDGVEVHNAKTKPREEQYNSTYRVVSVLTVCLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTAY

SEQ ID NO: 60 - DNA, 750 bp, Artificial Sequence
  nucleic acid encoding D221N/C575A
aacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtctgcctccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacaacgtgtccctggtcatgtccgacacagctggcaccgcctactga